(19) United States
(12) Patent Application Publication
(10) Pub. No.: US 2025/0195591 A1
(43) Pub. Date: Jun. 19, 2025

(54) MORRETON VIRUSES AND METHODS OF USE

(71) Applicant: MAYO FOUNDATION FOR MEDICAL EDUCATION AND RESEARCH, Rochester, MN (US)

(72) Inventors: Mitesh J. BORAD, Paradise Valley, AZ (US); Bolni M. NAGALO, Tempe, AZ (US)

(21) Appl. No.: 18/849,044

(22) PCT Filed: Mar. 2, 2023
(86) PCT No.: PCT/US2023/063569

§ 371 Date: Sep. 20, 2024

Related U.S. Application Data
(60) Provisional application No. 63/322,428, filed on Mar. 22, 2022.

(51) Int. Cl. A61K 35/766; A61P 35/00; C12N 7/00

(52) U.S. Cl. CPC A61K 35/766; A61P 35/00; C12N 7/00; C12N 2760/20221; C12N 2760/20232; C12N 2760/20271

(57) ABSTRACT

This document relates to methods and materials for treating cancer. For example, Morreton viruses (MORVs; e.g., recombinant MORVs) and methods for using such MORVs as an oncolytic agent (e.g., to treat cancer) are provided.

### CROSS-REFERENCE TO RELATED APPLICATIONS

This application claims the benefit of U.S. Patent Application Ser. No. 63/322,428, filed on Mar. 22, 2022. The disclosure of the prior application is considered part of (and is incorporated by reference in) the disclosure of this application.

### STATEMENT REGARDING FEDERAL FUNDING

This invention was made with government support under CA195764 awarded by the National Institutes of Health. The government has certain rights in the invention.

### SEQUENCE LISTING

This application contains a Sequence Listing that has been submitted electronically as an XML file named “07039-2094WO1.xml.” The XML file, created on Feb. 22, 2023, is 35,800 bytes in size. The material in the XML file is hereby incorporated by reference in its entirety.

### TECHNICAL FIELD

This document relates to methods and materials for treating cancer. For example, this document provides Morreton viruses (MORVs; e.g., recombinant MORVs) and methods for using such MORVs as an oncolytic agent (e.g., to treat cancer).

### BACKGROUND INFORMATION

Numerous oncolytic virus species have been reported to kill cancer cells while leaving normal tissues intact (Kaufman et al., Nat. Rev. Drug Discov., 15:660 (2016)). For example, the vesicular stomatitis virus (VSV) member of the genus Vesiculovirus in the family Rhabdoviridae has been studied for its therapeutic potention (Liu et al., Pathogens, 10(9):1092 (2021); and Bishnoi et al., Viruses, 10(2):90 (2018)). In spite of the therapeutic potential of VSV, its clinical translation has encountered a number of challenges, including potential for neurotoxicity and liver toxicity (Muik et al., Cancer Res., 74:3567-3578 (2014); Naik et al., Cancer Gene Ther., 19(7):443-450 (2012); Johnson et al., Virology, 360:36-49 (2007); and Zhang et al., Hum. Gene Ther Clin. Dev., 27:111-122 (2016)). In recent years, few other Vesiculovirus vectors have been investigated to overcome these challenges, and most have failed to achieve the level of potency of VSV in preclinical models of human cancers (Zemp et al., Biotechnol. Genet. Eng. Rev., 34:122-138 (2018)).

### SUMMARY

MORV is a new member of the genus Vesiculovirus that is non-pathogenic and close in phylogeny to, but antigenically distinct from, VSV (Walker et al., PLoS Pathog., 11:e1004664 (2015); and Amarasinghe et al., Arch. Virol., 162:2493-2504 (2017)). As described herein, MORVs can be used as an oncolytic viral platform for safe and effective oncolytic virotherapy.

This document provides methods and materials for treating cancer. For example, this document provides MORVs (e.g., recombinant MORVs) having oncolytic activity. In some cases, one or more MORVs described herein (e.g., one or more MORVs having oncolytic activity) can be used as an oncolytic agent (e.g., to treat cancer). For example, one or more MORVs (e.g., recombinant MORVs) described herein can be administered to a mammal having cancer to treat that mammal.

As demonstrated herein, MORVs can be used as a safe and effective oncolytic virotherapy to treat cancer (e.g., liver cancers such as cholangiocarcinoma and hepatocellular carcinoma). In some cases, wild-type MORV can be used to induce tumor regression in liver cancers (e.g., cholangiocarcinomas and hepatocellular carcinoma) without adverse events via immune-mediated and immune-independent mechanisms. In some cases, MORVs containing a recombinant genome having (a) a leader sequence of a Vesiculovirus species other than MORV, (b) at least one synthetic intergenic region, (c) a trailer sequence of a Vesiculovirus species other than MORV, or (d) any combination of two or three of (a)-(c) can be used to induce tumor regression in liver cancers (e.g., cholangiocarcinomas and hepatocellular carcinoma) without adverse events via immune-mediated and immune-independent mechanisms. Accordingly, one or more MORVs described herein (e.g., one or more recombinant MORVs) and/or nucleic acid encoding a MORV provided herein (e.g., nucleic acid encoding a recombinant MORV) can be used as anticancer agents to reduce the number of cancer cells within a mammal (e.g., a human).

In general, one aspect of this document features recombinant MORVs. The recombinant MORVs can include, or consist essentially of, (a) a leader sequence of a first Vesiculovirus species different from the MORV; (b) a synthetic intergenic region; and (c) a trailer sequence of a second Vesiculovirus species different from the MORV. The first Vesiculovirus species can be a VSV. The leader sequence can include the sequence set forth in SEQ ID NO:3. The leader sequence can consists of the sequence set forth in SEQ ID NO:3. The leader sequence can be located before a MORV-N gene within the MORV genome. The synthetic intergenic region can include a sequence set forth in SEQ ID NO:1 or SEQ ID NO:2. The synthetic intergenic region can consist of the sequence set forth in SEQ ID NO:2. The synthetic intergenic region can be located after a stop of a first gene within the MORV genome and before a start codon of a second gene within the MORV genome. The synthetic intergenic region can be located between each gene within the MORV genome. The second Vesiculovirus species can be a VSV. The trailer sequence can include a sequence set forth in SEQ ID NO:4. The trailer sequence can consist of the sequence set forth in SEQ ID NO:4. The trailer sequence can be located after a MORV-L gene within the MORV genome. The genome of the recombinant MORV can include: (a) the leader sequence before a MORV-N gene within the genome; (b) the synthetic intergenic region (i) after a stop codon of the MORV-N gene and before a start codon of a MORV-P gene, (ii) after a stop codon of the MORV-P gene and before a start codon of a MORV-M gene, (iii) after a stop codon of the MORV-M gene and before a start codon of a MORV-G gene, (iv) after a stop codon of the MORV-G gene and before a start codon of a MORV-L gene, and (v) after a stop codon of a MORV-L gene; and (c) the trailer sequence after the MORV-L gene. The genome of the recombinant MORV can include the nucleic acid sequence set forth in SEQ ID NO:5. The genome of the recombinant MORV can include the nucleic acid sequence set forth in SEQ ID NO:6.

In another aspect, this document features methods for treating a mammal having cancer. The methods can include, or consist essentially of, administering, to a mammal having cancer, a composition comprising MORV, wherein the number of cancer cells within the mammal is reduced. The mammal can be a human. The cancer can be a liver cancer, a pancreatic cancer, a breast cancer, a prostate cancer, a bladder cancer, a colorectal cancer, a lungs cancer, a thyroid cancer, a melanoma, a myeloma, or a sarcoma. The liver cancer can be a cholangiocarcinoma or a hepatocellular carcinoma. The MORV can be a wild-type MORV. The MORV can be a recombinant MORV. The recombinant MORV can include: (a) a leader sequence of a first Vesiculovirus species different from the MORV; (b) a synthetic intergenic region; and (c) a trailer sequence of a second Vesiculovirus species different from the MORV. The first Vesiculovirus species can be a VSV. The leader sequence can include the sequence set forth in SEQ ID NO:3. The leader sequence can consist of the sequence set forth in SEQ ID NO:3. The leader sequence can be located before a MORV-N gene within the MORV genome. The synthetic intergenic region can include a sequence set forth in SEQ ID NO:1 or SEQ ID NO:2. The synthetic intergenic region can consist of the sequence set forth in SEQ ID NO:2. The synthetic intergenic region can be located after a stop of a first gene within the MORV genome and before a start codon of a second gene within the MORV genome. The synthetic intergenic region can be located between each gene within the MORV genome. The second Vesiculovirus species can be a VSV. The trailer sequence can include a sequence set forth in SEQ ID NO:4. The trailer sequence can consist of the sequence set forth in SEQ ID NO:4. The trailer sequence can be located after a MORV-L gene within the MORV genome. The genome of the recombinant MORV can include: (a) the leader sequence before a MORV-N gene within the genome; (b) the synthetic intergenic region (i) after a stop codon of said MORV-N gene and before a start codon of a MORV-P gene, (ii) after a stop codon of the MORV-P gene and before a start codon of a MORV-M gene, (iii) after a stop codon of the MORV-M gene and before a start codon of a MORV-G gene, (iv) after a stop codon of the MORV-G gene and before a start codon of a MORV-L gene, and (v) after a stop codon of a MORV-L gene; and (c) the trailer sequence after the MORV-L gene. The genome of the recombinant MORV can include the nucleic acid sequence set forth in SEQ ID NO:5. The genome of the recombinant MORV can include the nucleic acid sequence set forth in SEQ ID NO:6.

In another aspect, this document features uses of compositions including MORVs descried herein (e.g., recombinant MORVs described herein) for treating a mammal having cancer.

In another aspect, this document features uses of compositions including MORVs descried herein (e.g., recombinant MORVs described herein) in the manufacture of a medicament for treating a mammal having cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B. Genome organization and ultrastructure of MORV. FIG. 1A: A schematic of a MORV, a negative-sense RNA Vesiculovirus, having a genome comprised of five major genes (MORV-N, MORV-P, MORV-M, MORV-G, and MORV-L). FIG. 1B: Transmission electron micrograph of MORV, a bullet-shaped virus about 200 nm in length and about 75 nm in width. Defective interfering (DI) particles designated as incomplete particles (IP) were found after several plaque purifications. G: MORV glycoprotein; VP: viral particle.

FIGS. 2A-2E. Characterization of MORV. FIG. 2A: Changes in titers of MORV and VSV grown in Vero cells over 48 hours. Recombinant MORV and laboratory-adapted VSV were used to infect BHK-21 cells. FIG. 2B: Supernatants from infected cells were collected at different time points, and viral titers were determined with a 50% tissue culture infective dose (TCID₅₀) method on Vero cells (1.5×10⁴). Right: Serial dilutions of MORV and VSV were used to infect a monolayer of Vero cells (5×10⁵). 48 hours later, cells were stained with crystal violet to reveal viral plaques. FIG. 2C: A549 (2×10⁴) IFN-responsive lung cancer cells were pretreated with various concentrations of universal type I IFN-α and then infected with viruses at MOI of 0.01. Cell viability was measured with a colorimetric assay (MTS) 48 hours post-infection. FIGS. 2D and 2E: Anti-VSV-G antibody, serum from patients treated with VSV (PT13-D2, PT14-22) expressing human IFN-β (VSV-hIFN-β), and normal human serum were evaluated for their ability to neutralize 400 TCID₅₀ units of MORV (FIG. 2D) and VSV (FIG. 2E) in Vero cells (2×10⁴). Cell viability was assessed 48 hours post-infection. Data are expressed as means of triplicate from three independent experiments.

FIGS. 3A-3B. Cytopathic effect of MORV and VSV in a panel of liver cancer cells. Monolayers (1.5×10⁵) of a transformed cholangiocyte cell line (H69), human CCA (EGI-1, PAX-42, HuCCT-1, SNU-1079, RBE, GBD-1, CAK-1, SNU-245, SNU-308, and SNU869), murine CCA (SB), human hepatocellular carcinoma (Hep3B, HepG2, Huh7, Sk-Hep1), and murine HCC (R1LWT, R2LWT, HCA-1 and Hepa 1-6) cells were mock-infected or were infected with MORV (FIG. 3A) or VSV (FIG. 3B) at an MOI of 0.01, 0.1, and 1. The percentage of cell viability was determined 72 hours post-infection by MTS assays (Promega, USA). The average of three independent experiments was plotted.

FIGS. 4A-4C. Assessment of MORV and VSV infectivity and cytotoxicity in low density lipoprotein receptor knockout (LDLR-KO) cell line (HAP-1). FIG. 4A: Expression of LDLR was measured with flow cytometry using fluorescein (FITC) anti-human LDLR antibody (solid line) or an isotype control antibody (dashed line). FIG. 4B: HAP1 WT cells or LDLR KO cells (1×10⁴) were infected with different MOIs (10, 1, 0.1, 0.01, 0.001, or 0.0001) of MORV or VSV. 72 hours post-infection, cell viability was measured with MTS assay. FIG. 4C: Representative images of infected HAP1 WT cells, mock-infected HAP1 WT cells, and LDLR KO cells. Cells were fixed at 48 hours, and then stained with crystal violet (scale bar=200 μm). Data are shown as mean±SEM from three independent experiments.

FIGS. 5A-5D. Intranasal administration of MORV and VSV in immunocompetent mice. FIG. 5A and FIG. 5B: Body weights of mice treated with high-dose MORV (FIG. 5A) or VSV (FIG. 5B) at 1×10¹⁰ TCID₅₀/kg. FIG. 5C: Hematoxylin and eosin (H&E)-stained brain sections from mice treated with high-dose MORV or VSV (1×10¹⁰ TCID₅₀/kg). FIG. 5D: Complete blood count (white blood cells (WBC, top panel) and lymphocytes (bottom panel)) values after intranasal administration of increasing doses (1×10⁷, 1×10⁸, 1×10⁹, and 1×10¹⁰ TCID₅₀/kg) of MORV or VSV.

FIGS. 6A-6D. In vivo antitumor efficacy of MORV and VSV in xenograft mouse models of cholangiocarcinoma (CCA) and hepatocellular carcinoma (HCC). One or two doses of MORV or VSV (1×10⁷ TCID₅₀) were injected intratumorally into mice bearing tumors initiated with HuCCT-1 cells (CCA) or Hep3B cells (HCC). Effects of MORV and VSV on tumor growth and tumor inhibition in HuCCT-1 xenografts (FIG. 6A and FIG. 6B) and in Hep3B xenografts (FIG. 6C and FIG. 6D).

FIGS. 7A-7G. Reduction of tumor burden in a mouse model of CCA required a lower dose of MORV than of VSV. FIG. 7A: Overview of the in vivo experiment. FIG. 7B: Weights of tumors from mice treated with PBS, MORV, or VSV at 1×10⁷ TCID₅₀ or 1×10⁸ TCID₅₀. FIG. 7C: Frequency of tumor-infiltrating CD3⁺CD8⁺ cytotoxic T lymphocytes (CTLs). FIGS. 7D-7G: Changes in serum ALP, ALT, IgM, and IgG2b. Triangle: PBS; squares: MORV; and circles: VSV.

FIGS. 8A-8B. Glycoprotein-based phylogeny of MORV. FIG. 8A: Phylogenic tree based on MORV glycoprotein and other viruses close to, yet distinct from, Vesiculoviruses. The Maximum Likelihood method and JTT matrix-based model were used. FIG. 8B: List of glycoproteins of known Vesiculoviruses genetically close to VSV.

FIGS. 9A-9B. MORV and VSV sensitivity to human type I interferon (IFN). FIG. 9A: Phase-contrast microscopy images of A549 cells treated with human IFN-α and infected with MORV and VSV at a multiplicity of infection (MOI) of 0.01 for 48 hours post-infection. FIG. 9B: A panel of human tumor cells (2×10⁴) was infected with MORV or VSV at MOI of 5. Levels of IFN-β in the supernatants of infected cells 18 hours after infection were measured. The average of three independent experiments was plotted.

FIG. 10. Intranasal administration of MORV and VSV are well tolerated in laboratory mice. Changes in blood parameters, including monocyte counts, granulocyte counts, red cell distribution width (RWA), hemoglobin (HGB) amount, red blood cell (RBC) counts, and platelet counts after intranasal treatments with MORV or VSV.

FIG. 11. Quantitation of viral nucleoprotein gene mRNA in mouse tissues after infection with MORV or VSV. Quantification of viral nucleoprotein gene (MORV-N or VSV-N) in the brain, blood, liver, and spleen tissues of immunocompetent mice treated with intranasal doses of MORV or VSV. Dosage: TCID₅₀/kg; ND: Non-detected or <1000 copies per ng of total RNA.

FIG. 12A-12B. Individual tumor volume and body weight (tumor xenografts). Graphs showing the effect of treatment with single or multiple doses of MORV or VSV on body weights of individual mice with HuCCT-1 xenografts (FIG. 12A) or Hep3B xenografts (FIG. 12B).

FIG. 13A-13B. Real-time analysis of MORV-induced apoptosis in vitro and quantification of apoptotic tumor cells from in vivo. FIG. 13A: Murine cholangiocarcinoma (SB) cells (1×10⁴ cells per well) were infected with MORV or VSV at different MOIs (10, 1, 0.1, or 0.01). Apoptosis was measured every 6 hours for 48 hours with Annexin V Red in the IncuCyte® S3 system (Essen Bioscience). Images were taken 48 hours after infection (MOI of 10). Data plotted as mean±SEM. FIG. 13B: Terminal deoxynucleotidyl transferase dUTP nick-end labeling (TUNEL) assays were used to analyze apoptosis of indicated cell samples from syngeneic tumor model of CCA after treatment with indicated doses of MORV or VSV.

FIGS. 14A-14C. Tumor nodules, liver function tests, and expression of MORV and VSV genes in orthotopic murine model of CCA. FIG. 14A: Representative images of tumor nodules in the control (PBS), MORV, and VSV groups. FIG. 14B: Analysis of serum levels of type I interferon (INF-α and INF-β) and biochemical parameters of blood (bilirubin, albumin, blood urea nitrogen, and cholesterol). FIG. 14C: Quantification of viral nucleoprotein (MORV-N and VSV-N) mRNA in tumor nodules after 4 weeks of treatment with MORV or VSV.

FIG. 15. MORV promotes greater CTL infiltration in murine CCA tumor microenvironment. Immune profiling of syngeneic mice that were administered PBS or viruses (MORV or VSV) showing percentages of tumor-infiltrating immune cells, including tumor-associated macrophages (TAMs), myeloid-derived suppressor cells (M-MDSCs), granulocytic myeloid-derived suppressor cells (G-MDSCs), programmed cell death protein 1 (PD1*), and granzyme (GzmB⁺) reactive CTLs, and natural killer (NK) cells. Unpaired t-test and one-way ANOVA were used to establish significance (p=0.05).

FIG. 16. Analysis of virus-specific immunoglobulin levels of MORV compared to VSV. Analysis of the immunoglobulin profiles of syngeneic mice that were administered intraperitoneal injections of PBS or indicated doses of MORV or VSV. Unpaired t-test and one-way ANOVA were used to determine significance (p=0.05).

FIGS. 17A-17C. Changes in mouse weight, spleen weight, and metastatic nodules following treatment with MORV and VSV. Changes in mouse weight, spleen weight, and metastatic nodules following treatment with MORV or VSV. Evaluation of changes in body weight (FIG. 17A), spleen weight (FIG. 17B), and tumor metastasis in the intestine (FIG. 17C) of mice treated with MORV or VSV.

### DETAILED DESCRIPTION

This document provides methods and materials for treating cancer. For example, this document provides methods and materials for treating cancer using one or more MORVs described herein (e.g., one or more recombinant MORVs) as an oncolytic agent (e.g., to treat cancer). In some cases, this document provides MORVs having oncolytic activity. For example, this document provides MORVs containing a recombinant genome having (a) a leader sequence of a Vesiculovirus species other than MORV, (b) at least one synthetic intergenic region, (c) a trailer sequence of a Vesiculovirus species other than MORV, or (d) any combination of two or three of (a)-(c). In some cases, a MORV provided herein can have a recombinant genome having a leader sequence of a Vesiculovirus species other than MORV and at least one synthetic intergenic region. In some cases, a MORV provided herein can have a recombinant genome having a leader sequence of a Vesiculovirus species other than MORV and a trailer sequence of a Vesiculovirus species other than MORV. In some cases, a MORV provided herein can have a recombinant genome having at least one synthetic intergenic region and a trailer sequence of a Vesiculovirus species other than MORV. In some cases, a MORV provided herein can have a recombinant genome having a leader sequence of a Vesiculovirus species other than MORV, at least one synthetic intergenic region, and a trailer sequence of a Vesiculovirus species other than MORV.

The MORVs described herein can have oncolytic activity. In some cases, this document provides methods for using one or more MORVs described herein (e.g., one or more recombinant MORVs) to treat a mammal having cancer. For example, one or more MORVs described herein (e.g., one or more recombinant MORVs) can be administered to a mammal (e.g., a human) having cancer to reduce the number of cancer cells (e.g., by infecting and killing cancer cells) in that mammal. In some cases, one or more MORVs described herein (e.g., one or more recombinant MORVs) can be administered to a mammal (e.g., a human) having cancer to stimulate anti-cancer immune responses in that mammal.

In some cases, a MORV provided herein (e.g., a recombinant MORV) can be replication competent.

In some cases, a MORV provided herein (e.g., a recombinant MORV) can infect dividing cells (e.g., can infect only dividing cells).

In some cases, a MORV provided herein (e.g., a recombinant MORV) can be non-pathogenic (e.g., to a mammal being treated as described herein).

In some cases, a MORV provided herein (e.g., a recombinant MORV) can be non-neurotropic (e.g., to a mammal being treated as described herein).

In some cases, a MORV provided herein (e.g., a recombinant MORV) can bud through the endoplasmic reticulum.

In some cases, a MORV provided herein (e.g., a recombinant MORV) can bind to a cellular receptor (e.g., to facilitate viral entry to a cell).

In some cases, a MORV provided herein (e.g., a recombinant MORV) can have reduced or eliminated neurotoxicity (e.g., as compared to another Vesiculovirus such as a VSV).

In some cases, a MORV provided herein (e.g., a recombinant MORV) can have reduced or eliminated hepatotoxicity (e.g., as compared to another Vesiculovirus such as a VSV).

In some cases, a MORV provided herein (e.g., a recombinant MORV) can have an increased oncolytic activity (e.g., as compared to another Vesiculovirus such as a VSV).

In some cases, a MORV provided herein (e.g., a recombinant MORV) may not be recognized (e.g., recognized and inactivated) by a neutralizing antibody. For example, a MORV provided herein (e.g., a recombinant MORV) may not be neutralized by a VSV neutralizing antibody present in a mammal having a pre-existing adaptive immunity to a VSV.

Any appropriate MORV can be used as described herein (e.g., as an oncolytic agent to treat a mammal having cancer). For example, any appropriate MORV can be used to create a recombinant MORV provided herein. When a MORV provided herein is a recombinant MORV, the recombinant MORV can be derived from (e.g., can include genomic elements such as nucleic acids encoding a polypeptide (or fragments thereof)) from any appropriate MORV. In some cases, a MORV that can be used to treat a mammal (e.g., a human) having cancer can include, or can be derived from, a genome having a nucleic acid sequence set forth in the National Center for Biotechnology Information (NCBI) databases in, for example, Accession No. NC_034508.1 or Accession No. KM205007.1. In some cases, a MORV that can be used to treat a mammal (e.g., a human) having cancer can be as described in Example 1. In some cases, a MORV that can be used to treat a mammal (e.g., a human) having cancer can be as described in Example 2.

AMORV provided herein (e.g., a recombinant MORV) can be any appropriate size. For example, a MORV provided herein (e.g., a recombinant MORV) can be about 100 nm in length (e.g., across its longest dimension).

In some cases, a MORV provided herein (e.g., a recombinant MORV) can include one or more nucleotide sequences that do not naturally occur in that MORV (e.g., do no naturally occur in that MORV prior to recombination). Nucleotide sequences that do not naturally occur in the MORV can be from any appropriate source. In some cases, a nucleotide sequence that does not naturally occur in that MORV can be from a non-viral organism. In some cases, a nucleotide sequence that does not naturally occur in a MORV provided herein can be from a virus other than a MORV. In some cases, a nucleotide sequence that does not naturally occur in that MORV can be from a MORV obtained from a different species (e.g., another species of Vesiculovirus such as VSV). In some cases, a nucleotide sequence that does not naturally occur in that MORV can be from a different strain of MORV (e.g., stereotypically distinct strains). In some cases, a nucleotide sequence that does not naturally occur in that MORV can be a synthetic nucleotide sequence.

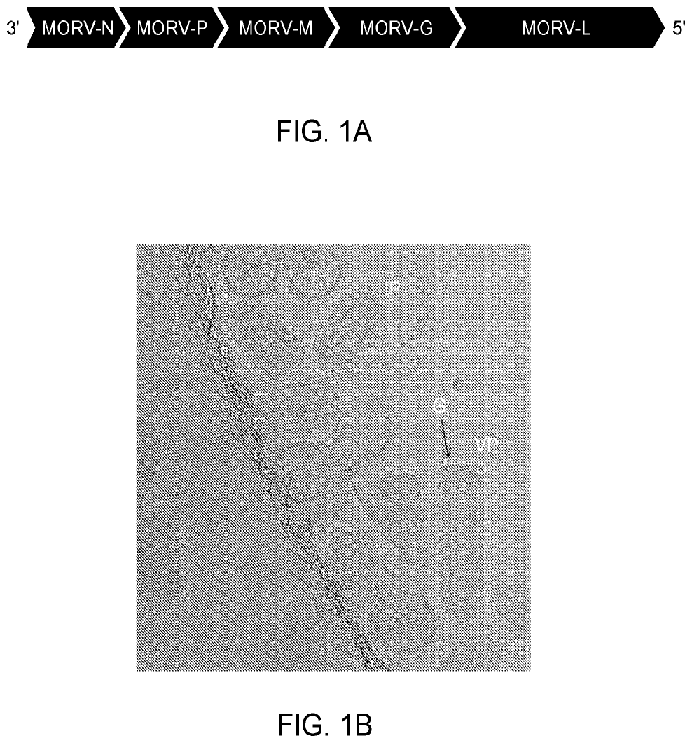

Nucleic acids that can be included in a MORV genome include, for example, MORV N gene (e.g., nucleic acid encoding a MORV nucleoprotein), MORV-P gene (e.g., nucleic acid encoding a MORV phosphoprotein), MORV-M gene (e.g., nucleic acid encoding a MORV matrix protein), MORV-G gene (e.g., nucleic acid encoding a MORV glycoprotein), and MORV-L gene (e.g., nucleic acid encoding a MORV polymerase). Viral elements that can be included in a MORV genome include, without limitation, leader sequences, 5′ long terminal repeats (LTRs), a 3′ LTRs, intergenic regions, and trailer sequences. A schematic of an exemplary MORV genome is shown in FIG. 1A.

In some cases, a MORV provided herein (e.g., a recombinant MORV) can include a chimeric MORV genome. For example, a chimeric MORV genome can include one or more nucleic acid sequences (e.g., one or more nucleic acids encoding a polypeptide (or fragments thereof) and/or one or more viral elements) from two or more (e.g., two, three, four, five, or more) different viral genomes. Examples of virus genomes from which one or more nucleic acid sequences in a chimeric MORV genome provided herein can be derived include, without limitation, a VSV genome, a Maraba virus genome, a Carajas virus genome, an Isfahan virus genome, a Radi virus genome, a Piry virus genome, a Malpais spring virus genome, and a Cocal virus genome. In some cases, a recombinant MORV provided herein can include one or more nucleic acid sequences from a VSV genome. For example, a recombinant MORV described herein can include one or more nucleic acid sequences from a VSV Indiana strain genome. A VSV Indiana strain can have a sequence set forth in the NCBI databases in, for example, Accession No. MW760865.1 or Accession No. J02428.1.

When a MORV provided herein (e.g., a recombinant MORV) includes one or more nucleotide sequences that do not naturally occur in that MORV (e.g., do no naturally occur in that MORV prior to recombination), the MORV can include any appropriate type of nucleotide sequence. In some cases, a MORV provided herein (e.g., a recombinant MORV) can include one or more intergenic regions (e.g., one or more intergenic regions designed to facilitate proper translation of one or more viral polypeptides). For example, a MORV provided herein (e.g., a recombinant MORV) can include one or more synthetic intergenic regions. Examples of intergenic regions that can be present in a genome of a MORV provided herein include, without limitation, intergenic regions comprising the sequence 5′-AACAGnnATC-3′ (SEQ ID NO:1; e.g., AACAGCAATC (SEQ ID NO:16)) and intergenic regions comprising the sequence 5′-TATGAAAAAAA-3′ (SEQ ID NO:2). In some cases, a MORV provided herein (e.g., a recombinant MORV) designed to include one or more intergenic regions can exhibit improved translation of one or more viral polypeptides. In some cases, a MORV provided herein (e.g., a recombinant MORV) designed to include one or more intergenic regions can exhibit enhanced viral replication. In some cases, a MORV provided herein (e.g., a recombinant MORV) designed to include one or more intergenic regions can spread into one or more tumor tissues.

In some cases, a MORV provided herein (e.g., a recombinant MORV) can include one or more leader sequences (e.g., one or more leader sequences that do not naturally occur in that MORV). For example, a MORV provided herein (e.g., a recombinant MORV) can include one or more leader sequences from a VSV Indiana strain genome, a Maraba virus genome, a Carajas virus genome, an Isfahan virus genome, a Radi virus genome, a Piry virus genome, a Malpais spring virus genome, or a Cocal virus genome. Examples of leader sequences that can be present in a genome of a MORV provided herein include, without limitation, leader sequences comprising the sequence 5′-AGACAAACAAACCATTATTACAATTAAAAGGCTCAGGAGAACCTTCAACAGCAAT CGAA-3′ (SEQ ID NO:3). In some cases, a MORV provided herein (e.g., a recombinant MORV) designed to include one or more leader sequences can exhibit proper viral RNA polymerase RNA dependent binding and activity. In some cases, a MORV provided herein (e.g., a recombinant MORV) designed to include one or more leader sequences can exhibit improved translation of one or more viral polypeptides.

In some cases, a MORV provided herein (e.g., a recombinant MORV) can include one or more trailer sequences (e.g., one or more trailer sequences that do not naturally occur in that MORV). For example, a MORV provided herein (e.g., a recombinant MORV) can include one or more trailer sequences from a VSV Indiana strain genome, a Maraba virus genome, a Carajas virus genome, an Isfahan virus genome, a Radi virus genome, a Piry virus genome, a Malpais spring virus genome, or a Cocal virus genome. Examples of trailer sequences that can be present in a genome of a MORV provided herein include, without limitation, trailer sequences comprising the sequence 5′-GATAAGACTTAGAACCCTCTTAGGATTTTTTTTGTTTTAAATGGTTTGTTGGTTTGG CATGGCATCTCCACC-3′ (SEQ ID NO:4). In some cases, a MORV provided herein (e.g., a recombinant MORV) designed to include one or more trailer sequences can exhibit improved viral replication. In some cases, a MORV provided herein (e.g., a recombinant MORV) designed to include one or more trailer sequences can exhibit improved viral kinetics.

When a MORV provided herein (e.g., a recombinant MORV) includes one or more nucleotide sequences that do not naturally occur in that MORV (e.g., do no naturally occur in that MORV prior to recombination), the nucleotide sequence(s) can be in any appropriate location within the MORV genome. In cases where an intergenic region (e.g., a synthetic intergenic region) is present in a MORV provided herein, the intergenic region can be present before a start codon and/or before a stop codon of a gene within the MORV genome. In some cases, an intergenic region can be present between each gene within the MORV genome. For example, an intergenic region (e.g., a synthetic intergenic region) can be located after a stop codon of a first MORV gene within the MORV genome and before a start codon of a second MORV gene within the MORV genome. In some cases, a MORV provided herein (e.g., a recombinant MORV) can include a synthetic intergenic region located between each gene in the MORV genome.

In cases where a leader sequence (e.g., a chimeric leader sequence such as a leader sequence from a VSV Indiana strain genome) is present in a MORV provided herein, the leader sequence can be present before a gene within the MORV genome. For example, a leader sequence can be present before a MORV-N gene within the MORV genome.

In cases where a trailer sequence (e.g., a chimeric trailer sequence such as a trailer sequence from a VSV Indiana strain genome) is present in a MORV provided herein, the trailer sequence can be present after a gene within the MORV genome. For example, a trailer sequence can be present after a MORV-L gene within the MORV genome.

In some cases, a recombinant MORV provided herein (e.g., a recombinant MORV having oncolytic activity) can include a leader sequence (e.g., a chimeric leader sequence such as a leader sequence from a VSV Indiana strain genome) before a MORV-N gene, an intergenic region (e.g., a synthetic intergenic region) between a MORV-N gene and a MORV-P gene, an intergenic region (e.g., a synthetic intergenic region) between a MORV-P gene and a MORV-M gene, an intergenic region (e.g., a synthetic intergenic region) between a MORV-M gene and a MORV-G gene, an intergenic region (e.g., a synthetic intergenic region) between a MORV-G gene and a MORV-L gene, an intergenic region (e.g., a synthetic intergenic region) after a MORV-L gene, and a trailer sequence after a MORV-L gene (e.g., after an intergenic region that is after a MORV-L gene). For example, a recombinant MORV provided herein can include a leader sequence (e.g., a chimeric leader sequence such as a leader sequence from a VSV Indiana strain genome) before a start codon of a MORV-N gene, an intergenic region (e.g., a synthetic intergenic region) after a stop codon of a MORV-N gene and before a start codon of a MORV-P gene, an intergenic region (e.g., a synthetic intergenic region) after a stop codon of a MORV-P gene and before a start codon of a MORV-M gene, an intergenic region (e.g., a synthetic intergenic region) after a stop codon of a MORV-M gene and before a start codon of a MORV-G gene, an intergenic region (e.g., a synthetic intergenic region) after a stop codon of a MORV-G gene and before a start codon of a MORV-L gene, an intergenic region (e.g., a synthetic intergenic region) after a stop codon of a MORV-L gene, and a trailer sequence after a MORV-L gene.

In some cases, a MORV (e.g., a recombinant MORV) having oncolytic activity can include a genome having a nucleic acid sequence as set forth in SEQ ID NO:5.

In some cases, a MORV (e.g., a recombinant MORV) having oncolytic activity can include a genome having a nucleic acid sequence as set forth in SEQ ID NO:6.

In some cases, a MORV provided herein (e.g., a recombinant MORV) can include a genome containing a sequence that deviates from the nucleotide sequence set forth in SEQ ID NO:5 or SEQ ID NO:6, sometimes referred to as a variant sequence. For example, a nucleotide sequence of a genome of a MORV can have at least 80% sequence identity (e.g., about 82% sequence identity, about 85% sequence identity, about 88% sequence identity, about 90% sequence identity, about 93% sequence identity, about 95% sequence identity, about 97% sequence identity, about 98% sequence identity, or about 99% sequence identity) to the nucleotide sequence set forth in SEQ ID NO:5 or SEQ ID NO:6. Percent sequence identity is calculated by determining the number of matched positions in aligned nucleotide sequences, dividing the number of matched positions by the total number of aligned nucleotide, and multiplying by 100. A matched position refers to a position in which identical nucleotide occur at the same position in aligned sequences. Sequences can be aligned using the algorithm described by Altschul et al. (Nucleic Acids Res., 25:3389-3402 (1997)) as incorporated into BLAST (basic local alignment search tool) programs, available at ncbi.nlm.nih.gov on the World Wide Web. BLAST searches or alignments can be performed to determine percent sequence identity between a nucleic acid and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a nucleotide sequence and another sequence, the default parameters of the respective programs can be used.

In some cases, a MORV provided herein (e.g., a recombinant MORV) can include a MORV genome containing a nucleic acid sequence that has one or more (e.g., one, two, three, four, five, six, seven, eight, nine, ten, or more) modifications (e.g., as compared to the nucleotide sequence set forth in SEQ ID NO:5 or SEQ ID NO:6). For example, a MORV provided herein can have one or more modifications as compared to a MORV that includes a genome having the nucleic acid sequence as set forth in SEQ ID NO:5. In another example, a MORV provided herein can have one or more modifications as compared to the MORV that includes a genome having the nucleic acid sequence as set forth in SEQ ID NO:6. A modification can be any type of modification. Examples of modifications that can be made to a nucleotide sequence include, without limitation, deletions, insertions, substitutions, or combinations thereof. In some cases, a modification can be a silent modification (e.g., a modification to nucleic acid encoding a polypeptide that does not produce a modification in the encoded polypeptide). In some cases, a modification can be in a nucleic acid encoding a polypeptide. In some cases, a modification can be in a viral element of the MORV genome.

Also provided herein are vectors (e.g., expression vectors) containing nucleic acid encoding a MORV provided herein (e.g., recombinant MORV). Vectors can carry nucleic acid encoding a MORV provided herein into another cell (e.g., a cancer cell), where it can be replicated and/or expressed. An expression vector, also commonly referred to as an expression construct, is typically a plasmid or vector having an enhancer/promoter region controlling expression of a specific nucleotide sequence. When introduced into a cell, the expression vector can use cellular protein synthesis machinery to produce the virus in the cell.

A vector containing nucleic acid encoding a MORV provided herein (e.g., recombinant MORV) can be any appropriate type of expression vector. In some cases, a vector can be a viral vector. In some cases, a vector can be a non-viral vector.

When a vector containing nucleic acid encoding a MORV provided herein (e.g., recombinant MORV) is a viral vector, any appropriate viral vector can be used. A viral vector can be derived from a positive-strand virus or a negative-strand virus. A viral vector can be derived from a virus with a DNA genome or a RNA genome. In some cases, a viral vector can be a chimeric viral vector. In some cases, a viral vector can infect dividing cells. In some cases, a viral vector can infect non-dividing cells. Examples virus-based vectors that can be used to deliver nucleic acid encoding a MORV provided herein (e.g., a recombinant MORV) to a mammal (e.g., a human) include, without limitation, virus-based vectors based on adenoviruses, AAVs, Sendai viruses, retroviruses, lentiviruses, paramyxoviruses, Coxsackieviruses, poxviruses, and herpes viruses.

When a vector containing nucleic acid encoding a MORV provided herein (e.g., recombinant MORV) is a non-viral vector, any appropriate non-viral vector can be used. In some cases, a non-viral vector can be an expression plasmid (e.g., a cDNA expression vector).

In addition to nucleic acid encoding a MORV provided herein (e.g., a recombinant MORV), a vector (e.g., a viral vector or a non-viral vector) can contain one or more regulatory elements operably linked to the nucleic acid encoding a MORV provided herein (e.g., a recombinant MORV). Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid encoding a MORV provided herein (e.g., a recombinant MORV). A promoter can be a naturally occurring promoter or a recombinant promoter. A promoter can be ubiquitous or inducible (e.g., in the presence of tetracycline), and can affect the expression of a nucleic acid encoding a polypeptide in a general or tissue-specific manner. Examples of promoters that can be used to drive expression of a MORV provided herein (e.g., a recombinant MORV) in cells include, without limitation, T7 promoters. As used herein, “operably linked” refers to positioning of a regulatory element in a vector relative to a nucleic acid encoding a polypeptide in such a way as to permit or facilitate expression of the encoded polypeptide. For example, a vector can contain a promoter and nucleic acid encoding a MORV provided herein (e.g., a recombinant MORV). In this case, the promoter is operably linked to a nucleic acid encoding a MORV provided herein (e.g., a recombinant MORV) such that it drives expression of the MORV in cells to produce the virus in the cell.

This document also provides methods and materials for using one or more MORVs provided herein (e.g., one or more recombinant MORVs). In some cases, one or more MORVs provided herein (e.g., one or more recombinant MORVs) can be used for treating a mammal (e.g., a human) having cancer. For example, one or more MORVs provided herein (e.g., one or more recombinant MORVs) and/or nucleic acid encoding a MORV provided herein (e.g., nucleic acid encoding a recombinant MORV) can be administered to a mammal having cancer to treat the mammal. In some cases, administering one or more MORVs provided herein (e.g., one or more recombinant MORVs) and/or nucleic acid encoding a MORV provided herein (e.g., nucleic acid encoding a recombinant MORV) to a mammal (e.g., a human) having cancer can increase survival of the mammal. In some cases, administering one or more MORVs provided herein (e.g., one or more recombinant MORVs) and/or nucleic acid encoding a MORV provided herein (e.g., nucleic acid encoding a recombinant MORV) to a mammal (e.g., a human) having cancer can stimulate an anti-cancer immune response in the mammal.

In some cases, one or more MORVs provided herein (e.g., one or more recombinant MORVs) and/or nucleic acid encoding a MORV provided herein (e.g., nucleic acid encoding a recombinant MORV) can be administered to a mammal to reduce the size of the cancer in the mammal. For example, one or more MORVs provided herein (e.g., one or more recombinant MORVs) and/or nucleic acid encoding a MORV provided herein (e.g., nucleic acid encoding a recombinant MORV) can be administered to a mammal (e.g., a human) in need thereof (e.g., a human having cancer) as described herein to reduce the number of cancer cells in the mammal by, for example, 10, 20, 30, 40, 50, 60, 70, 80, 90, 95, or more percent. For example, one or more MORVs provided herein (e.g., one or more recombinant MORVs) and/or nucleic acid encoding a MORV provided herein (e.g., nucleic acid encoding a recombinant MORV) can be administered to a mammal (e.g., a human) in need thereof (e.g., a human having cancer) as described herein to reduce the volume of one or more tumors in the mammal by, for example, 10, 20, 30, 40, 50, 60, 70, 80, 90, 95, or more percent.

In some cases, one or more MORVs provided herein (e.g., one or more recombinant MORVs) and/or nucleic acid encoding a MORV provided herein (e.g., nucleic acid encoding a recombinant MORV) can be administered to a mammal to induce oncolysis of cancer cells within a mammal. For example, one or more MORVs provided herein (e.g., one or more recombinant MORVs) and/or nucleic acid encoding a MORV provided herein (e.g., nucleic acid encoding a recombinant MORV) can be administered to a mammal to induce cell death in a cell of the mammal (e.g., in an infected cell of the mammal). For example, one or more MORVs provided herein (e.g., one or more recombinant MORVs) and/or nucleic acid encoding a MORV provided herein (e.g., nucleic acid encoding a recombinant MORV) can be administered to a mammal (e.g., a human) in need thereof (e.g., a human having cancer) as described herein to induce oncolysis in, for example, 10, 20, 30, 40, 50, 60, 70, 80, 90, 95, or more percent of the cancer cells in the mammal.

In some cases, one or more MORVs provided herein (e.g., one or more recombinant MORVs) and/or nucleic acid encoding a MORV provided herein (e.g., nucleic acid encoding a recombinant MORV) can be administered to a mammal to induce a cytopathic effect (CPE) in a cell of the mammal. For example, one or more MORVs provided herein (e.g., one or more recombinant MORVs) and/or nucleic acid encoding a MORV provided herein (e.g., nucleic acid encoding a recombinant MORV) can be administered to a mammal to induce syncytia formation of a cell of the mammal (e.g., of an infected cell of the mammal). For example, one or more MORVs provided herein (e.g., one or more recombinant MORVs) and/or nucleic acid encoding a MORV provided herein (e.g., a recombinant MORV) can be administered to a mammal to induce vacuolization of a cell of the mammal (e.g., of an infected cell of the mammal). For example, one or more MORVs provided herein (e.g., one or more recombinant MORVs) and/or nucleic acid encoding a MORV provided herein (e.g., nucleic acid encoding a recombinant MORV) can be administered to a mammal (e.g., a human) in need thereof (e.g., a human having cancer) as described herein to induce a CPE in, for example, 10, 20, 30, 40, 50, 60, 70, 80, 90, 95, or more percent of the cancer cells in the mammal.

Any appropriate mammal having, or at risk of having, cancer can be treated as described herein. Examples of mammals that can have, or can be at risk of having, cancer and can be treated as described herein (e.g., by administering one or more MORVs provided herein and/or nucleic acid encoding a MORV provided herein) include, without limitation, humans, non-human primates such as monkeys, horses, bovine species, porcine species, dogs, cats, mice, and rats. In some cases, a human having cancer can be treated as described herein. In some cases, a mammal (e.g., a human) treated as described herein is not a natural host of and/or does not have pre-existing immunity against a MORV.

A mammal having any type of cancer can be treated as described herein (e.g., by administering one or more MORVs provided herein and/or nucleic acid encoding a MORV provided herein). In some cases, a cancer treated as described herein can include one or more solid tumors. In some cases, a cancer treated as described herein can be a hematologic cancer (e.g., a blood cancer). Examples of cancers that can be treated as described herein include, without limitation, liver cancers (e.g., cholangiocarcinomas and hepatocellular carcinomas), pancreatic cancers, breast cancers, prostate cancers, bladder cancers, colorectal cancers, lung cancers, thyroid cancers, melanomas, myelomas, and sarcomas.

In some cases, methods described herein also can include identifying a mammal as having cancer. Examples of methods for identifying a mammal as having cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and/or genetic tests. Once identified as having cancer, a mammal can be administered or instructed to self-administer one or more MORVs provided herein (e.g., one or more recombinant MORVs) and/or nucleic acid encoding a MORV provided herein (e.g., nucleic acid encoding a recombinant MORV).

One or more MORVs provided herein (e.g., one or more recombinant MORVs) and/or nucleic acid encoding a MORV provided herein (e.g., nucleic acid encoding a recombinant MORV) can be administered by any appropriate route (e.g., intratumoral, intraperitoneal, intravenous, intramuscular, subcutaneous, oral, intranasal, inhalation, transdermal, and parenteral) to a mammal. In some cases, one or more MORVs provided herein (e.g., one or more recombinant MORVs) and/or nucleic acid encoding a MORV provided herein (e.g., nucleic acid encoding a recombinant MORV) can be administered intratumorally to a mammal (e.g., a human). In some cases, one or more MORVs provided herein (e.g., one or more recombinant MORVs) and/or nucleic acid encoding a MORV provided herein (e.g., nucleic acid encoding a recombinant MORV) can be administered intraperitoneally to a mammal (e.g., a human).

In some cases, one or more MORVs provided herein (e.g., one or more recombinant MORVs) and/or nucleic acid encoding a MORV provided herein (e.g., a recombinant MORV) can be formulated into a composition (e.g., a pharmaceutical composition) for administration to a mammal (e.g., a mammal having, or at risk of having, cancer). For example, one or more MORVs provided herein (e.g., one or more recombinant MORVs) and/or nucleic acid encoding a MORV provided herein (e.g., nucleic acid encoding a recombinant MORV) can be formulated into a pharmaceutically acceptable composition for administration to a mammal having, or at risk of having, cancer. In some cases, one or more MORVs provided herein (e.g., one or more recombinant MORVs) and/or nucleic acid encoding a MORV provided herein (e.g., nucleic acid encoding a recombinant MORV) can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline such as phosphate-buffered saline (PBS), protamine sulfate, disodium hydrogen phosphate, potassium hydrogen phosphate, sodium chloride, and zinc salts), colloidal silica, magnesium trisilicate, polyacrylates, waxes, wool fat, and lecithin.

In some cases, one or more MORVs provided herein (e.g., one or more recombinant MORVs) and/or nucleic acid encoding a MORV provided herein (e.g., a recombinant MORV) can be packaged into one or more carrier cells. For example, one or more MORVs provided herein (e.g., one or more recombinant MORVs) and/or nucleic acid encoding a MORV provided herein (e.g., nucleic acid encoding a recombinant MORV) can be formulated into carrier cells for administration to a mammal having, or at risk of having, cancer. Examples of cells that can be used as carrier cells for one or more MORVs provided herein and/or nucleic acid encoding a MORV provided herein include, without limitation, myeloid derived cells and NK cells.

A composition (e.g., a pharmaceutical composition) containing one or more MORVs provided herein (e.g., one or more recombinant MORVs) and/or nucleic acid encoding a MORV provided herein (e.g., nucleic acid encoding a recombinant MORV) can be formulated into any appropriate dosage form. Examples of dosage forms include solid or liquid forms including, without limitation, gums, capsules, tablets (e.g., chewable tablets, and enteric coated tablets), suppositories, liquids, enemas, suspensions, solutions (e.g., sterile solutions), sustained-release formulations, delayed-release formulations, pills, powders, and granules.

A composition (e.g., a pharmaceutical composition) containing one or more MORVs provided herein (e.g., one or more recombinant MORVs) and/or nucleic acid encoding a MORV provided herein (e.g., nucleic acid encoding a recombinant MORV) can be designed for oral or parenteral (including subcutaneous, intratumoral, intramuscular, intravenous, topical, and intradermal) administration. When being administered orally, a pharmaceutical composition containing one or more MORVs provided herein can be in the form of a pill, syrup, gel, liquid, flavored drink, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more MORVs provided herein (e.g., one or more recombinant MORVs) and/or nucleic acid encoding a MORV provided herein (e.g., nucleic acid encoding a recombinant MORV) can be administered locally or systemically. For example, a composition containing one or more MORVs provided herein (e.g., one or more recombinant MORVs) and/or nucleic acid encoding a MORV provided herein (e.g., nucleic acid encoding a recombinant MORV) can be administered locally by an intratumoral injection to a tumor within a mammal (e.g., a human). For example, a composition containing one or more MORVs provided herein (e.g., one or more recombinant MORVs) and/or nucleic acid encoding a MORV provided herein (e.g., nucleic acid encoding a recombinant MORV) can be administered systemically by an oral administration to a mammal (e.g., a human).

An effective amount of a composition (e.g., a pharmaceutical composition) containing one or more MORVs provided herein (e.g., one or more recombinant MORVs) and/or nucleic acid encoding a MORV provided herein (e.g., nucleic acid encoding a recombinant MORV) can be any amount that can treat the cancer without producing significant toxicity to the mammal. An effective amount of one or more MORVs provided herein (e.g., one or more recombinant MORVs) and/or nucleic acid encoding a MORV provided herein (e.g., nucleic acid encoding a recombinant MORV) can be any appropriate amount. In some cases, an effective amount of one or more MORVs provided herein (e.g., one or more recombinant MORVs) can be from about 1×10⁷ TCID₅₀ (tissue culture infective dose) per kg body weight of the mammal to be treated (TCID₅₀/kg) to about 1×10¹⁰ TCID₅₀/kg. In some cases, an effective amount of one or more MORVs provided herein (e.g., one or more recombinant MORVs) can be about 1×10⁷ TCID₅₀. In some cases, an effective amount of one or more MORVs provided herein (e.g., one or more recombinant MORVs) can be about 1×10⁸ TCID₅₀. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., a cancer) may require an increase or decrease in the actual effective amount administered.

The frequency of administration of a composition (e.g., a pharmaceutical composition) containing one or more MORVs provided herein (e.g., one or more recombinant MORVs) and/or nucleic acid encoding a MORV provided herein (e.g., nucleic acid encoding a recombinant MORV) can be any frequency that can treat the cancer without producing significant toxicity to the mammal. For example, the frequency of administration can be from about three times a day to about once a week, from about twice a day to about twice a week, or from about once a day to about twice a week. The frequency of administration can remain constant or can be variable during the duration of treatment. A course of treatment with a composition containing one or more MORVs provided herein can include rest periods. For example, a composition containing one or more MORVs provided herein (e.g., one or more recombinant MORVs) and/or nucleic acid encoding a MORV provided herein (e.g., nucleic acid encoding a recombinant MORV) can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., a cancer) may require an increase or decrease in administration frequency.

An effective duration for administering a composition (e.g., a pharmaceutical composition) containing one or more MORVs provided herein (e.g., one or more recombinant MORVs) and/or nucleic acid encoding a MORV provided herein (e.g., nucleic acid encoding a recombinant MORV) can be any duration that treat the cancer without producing significant toxicity to the mammal. For example, the effective duration can vary from several days to several weeks, months, or years. In some cases, the effective duration for the treatment of a cancer can range in duration from about one month to about 10 years. Multiple factors can influence the actual effective duration used for a particular treatment. For example, an effective duration can vary with the frequency of administration, effective amount, use of multiple treatment agents, route of administration, and severity of the condition being treated.

In some cases, one or more MORVs provided herein (e.g., one or more recombinant MORVs) and/or nucleic acid encoding a MORV provided herein (e.g., nucleic acid encoding a recombinant MORV) can be used as the sole active agent(s) to treat a mammal (e.g., a human) having cancer.

In some cases, one or more MORVs provided herein (e.g., one or more recombinant MORVs) and/or nucleic acid encoding a MORV provided herein (e.g., nucleic acid encoding a recombinant MORV) can be administered to a mammal (e.g., a human) having cancer together with one or more (e.g., one, two, three, four, five or more) additional agents/therapies used to treat cancer. Examples of additional anti-cancer agents that can be used in combination with a MORV provided herein (or a nucleic acid encoding a MORV provided herein) include, without limitation, chemotherapeutic agents, targeted therapies, cytotoxic agents, immune checkpoint inhibitors, anti-angiogenics, and any combinations thereof. In cases where one or more MORVs provided herein (e.g., one or more recombinant MORVs) and/or nucleic acid encoding a MORV provided herein (e.g., nucleic acid encoding a recombinant MORV) are used in combination with additional agents used to treat cancer, the one or more additional agents can be administered at the same time (e.g., in a single composition containing both one or more MORVs provided herein and/or nucleic acid encoding a MORV provided herein and containing the one or more additional agents) or independently. For example, one or more MORVs provided herein (e.g., one or more recombinant MORVs) and/or nucleic acid encoding a MORV provided herein (e.g., nucleic acid encoding a recombinant MORV) can be administered first, and the one or more additional agents administered second, or vice versa. Examples of therapies that can be used to treat cancer include, without limitation, surgery, radiation therapies, and adoptive cell transfer therapies. In cases where one or more MORVs provided herein (e.g., one or more recombinant MORVs) and/or nucleic acid encoding a MORV provided herein (e.g., nucleic acid encoding a recombinant MORV) are used in combination with one or more additional therapies used to treat cancer, the one or more additional therapies can be performed at the same time or independently of the administration of one or more MORVs provided herein (e.g., one or more recombinant MORVs) and/or nucleic acid encoding a MORV provided herein (e.g., nucleic acid encoding a recombinant MORV). For example, the one or more MORVs provided herein and/or nucleic acid encoding a MORV provided herein can be administered before, during, or after the one or more additional therapies are performed.

In some cases, the size of the cancer (e.g., the number of cancer cells and/or the volume of one or more tumors) present within a mammal and/or the severity of one or more symptoms of the cancer being treated can be monitored. Any appropriate method can be used to determine whether or not the size of the cancer present within a mammal is reduced. For example, imaging techniques can be used to assess the size of the cancer present within a mammal.

The invention will be further described in the following examples, which do not limit the scope of the invention described in the claims.

### EXAMPLES

### Example 1: Characterization of Morreton Virus (MORV) as a Novel Oncolytic Virotherapy Platform for Liver Cancers

This Example assesses the safety of MORV after intranasal administration, and evaluates antitumor efficacy of single and multiple doses of MORV. As described herein, MORV has comparable oncolytic potency to VSV and a better safety profile than VSV. As such, MORV can be used as an oncolytic agent (e.g., to treat cancer).

### Results

### Ultrastructure and Genome Sequence of Morreton Virus (MORV)

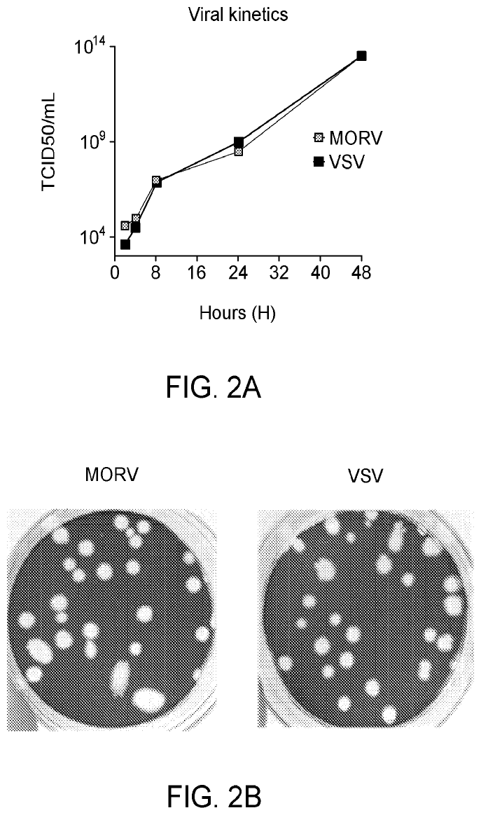

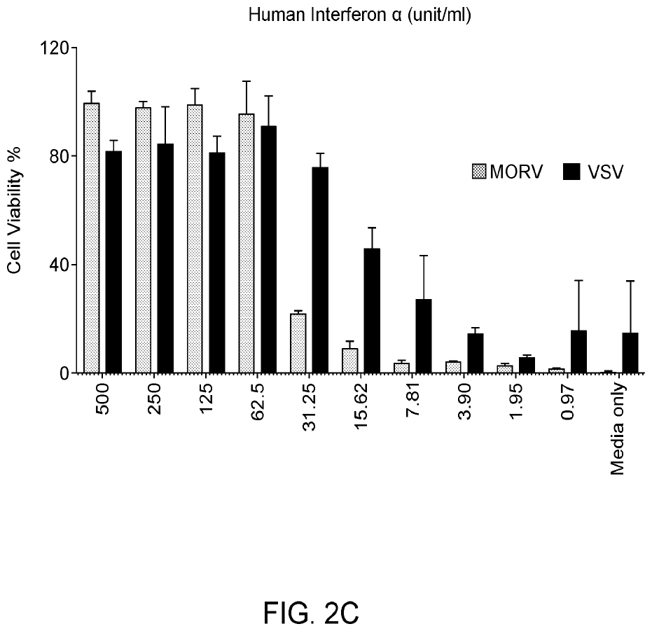

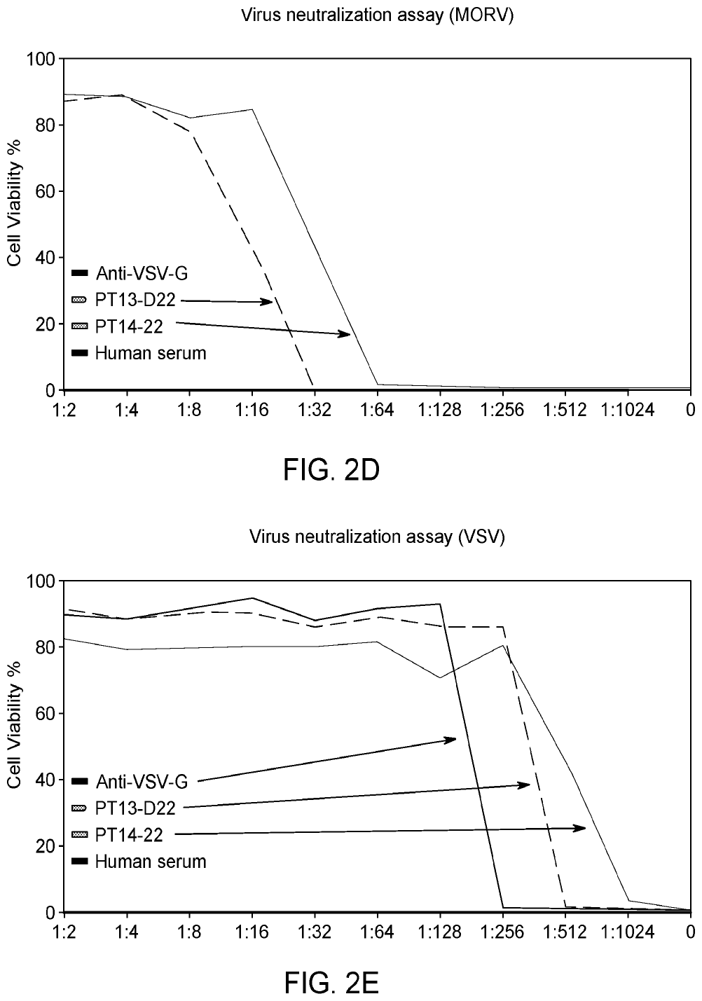

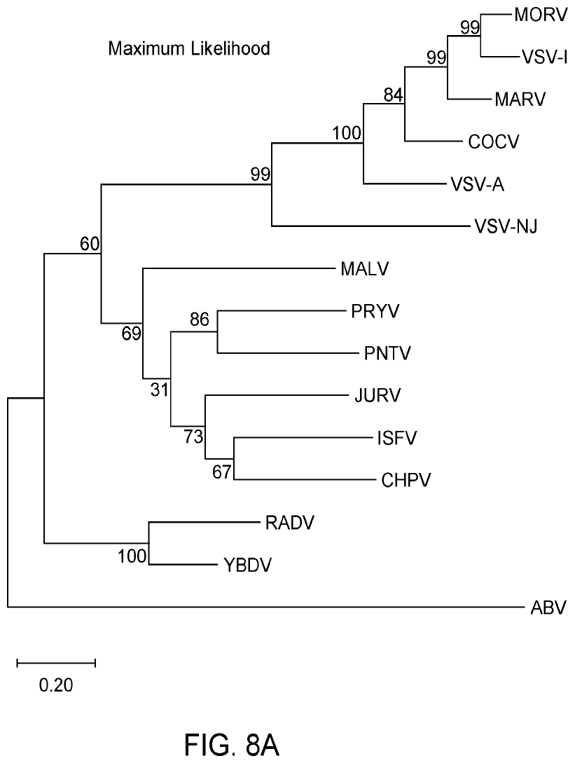

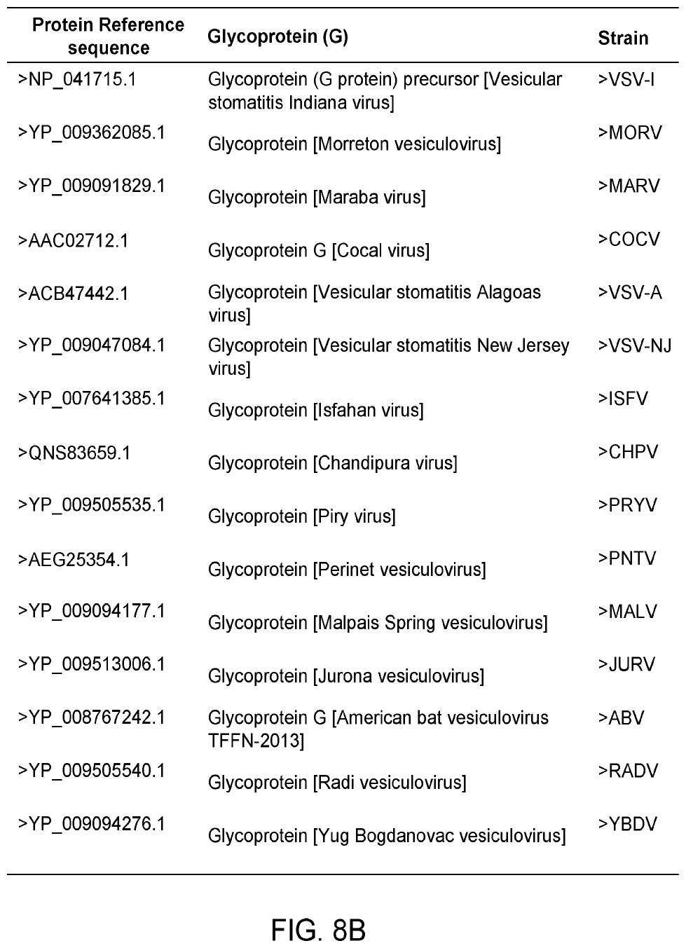

MORV is a nonpathogenic, bullet-shaped (length, 200 nm; width, 75 nm) negative-sense RNA Vesiculovirus of the family Rhabdoviridae (FIGS. 1A-1B). Analysis of glycoprotein (G protein) among members of this virus family to construct a phylogenetic tree indicates that MORV is closely related to yet genetically distinct from vesicular stomatitis virus Indiana strain (FIGS. 1A-1B; FIG. 8). The MORV genome is 11,181 bp in length and has five specific Vesiculovirus genes (from the 3′ to 5′ direction): nucleoprotein (MORV-N), phosphoprotein (MORV-P), matrix (MORV-M), glycoprotein (MORV-G), and polymerase (MORV-L). Although the infectivity of MORV was similar to that of VSV, MORV could be grown at comparably high titers and formed relatively larger plaques (FIGS. 2A-2B).

Oncolytic MORV is Sensitive to Anti-Viral Mechanisms Associated with Host Type Interferon (IFN-α) Response

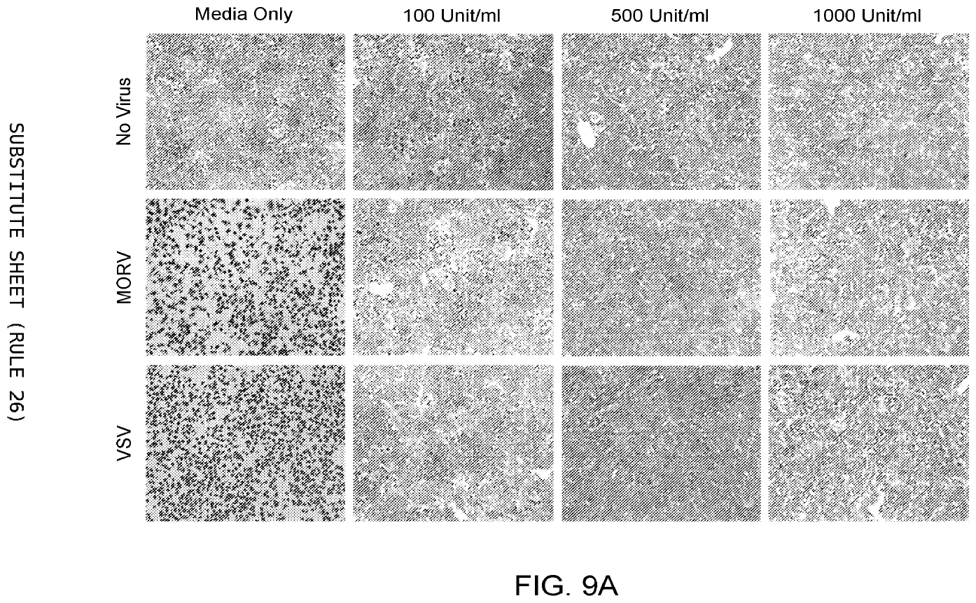

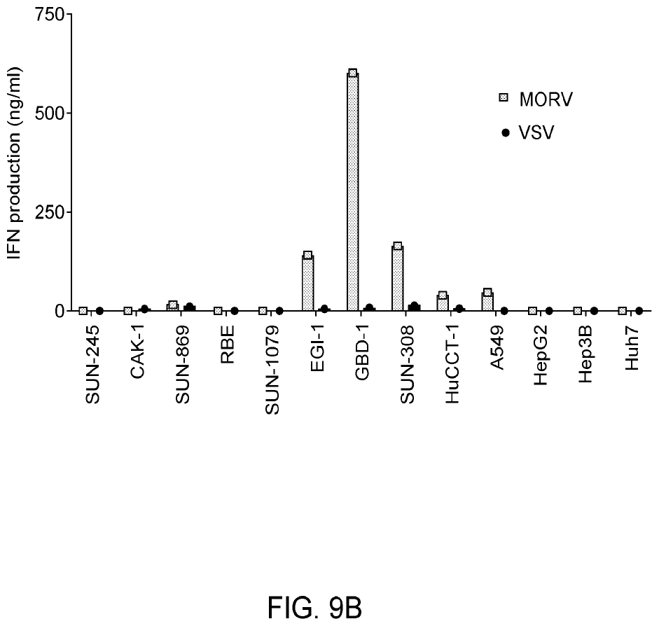

Mammalian cells respond to viral infection by expressing antiviral cytokines, such as type I IFN-α and IFNβ, and many oncolytic viruses selectively infect and replicate in tumor cells in which the functional IFN-associated antiviral responses are partially or completely impaired. To examine the impact of IFN on MORV replication, a monolayer of A549 IFN-responsive lung cancer cells was treated with serial concentrations of human type I IFN-α for 24 hours, and then infected with MORV or VSV at a multiplicity of infection (MOI) of 5. Dose-response treatment with MORV or VSV resulted in inhibition of MORV and VSV oncolysis (FIG. 2C), with differences in sensitivity to IFN-α (FIG. 9A). The lowest concentration of human IFN-α that significantly inhibited VSV-induced cytopathic effect (CPE) (75% cell viability) was 31.25 units/mL. MORV required a 2-fold higher dose (62.5 units/mL) to induce protection (85% cell viability). Overall, both MORV and VSV were sensitive to the antiviral effects of type I IFN-α. However, a low level of IFN-α did not significantly inhibit cytopathic effects induced by MORV, relative to those induced by VSV.

### MORV is Resistant to Anti-VSV-G Neutralizing Antibodies and to Normal Human Serum

MORV sensitivity to anti-VSV-G or other pre-existing antibodies in patients was evaluated by assessing the MORV-neutralizing ability of anti-VSV neutralizing antibodies (Anti-VSV-G), serum from patients treated with VSV-hIFN-β (ClinicalTrials.gov: NCT01628640), and normal pooled human serum. The data showed that, while the anti-VSV-G antibody effectively neutralized VSV, it was ineffective against MORV-induced CPE (FIGS. 2D-2E). However, serum samples obtained from patients treated with VSV-hIFN-β (PT13-D22, PT14-22) neutralized MORV, but only at elevated serum concentrations (FIG. 2D). Furthermore, serum from PT13-D22 (80% cell viability at 1:80 dilution) was slightly less potent than serum from PT14-22 (85% cell viability at 1:160 dilution) in its inhibition of MORV. Pooled samples of human serum failed to neutralize MORV or VSV, indicating low pre-existing immunity in the general population for these vectors.

Additionally, IFN-β was not quantifiable in serum from PT13-D22 and PT14-22, suggesting that VSV-hIFN-β did not extensively replicate in these patients. Therefore, the observed neutralization effects seen with patient serum are most likely due to polyclonal anti-VSV antibodies against viral epitopes common to VSV and MORV.

### MORV Induced Robust CPE in Tumor Cells

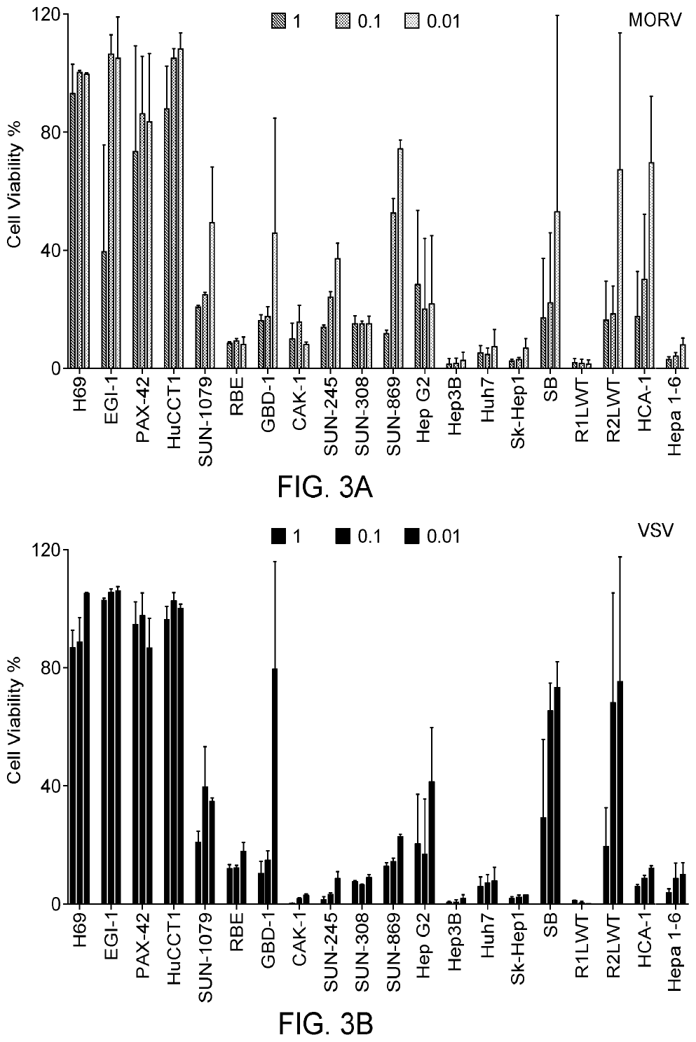

To evaluate the cytotoxicity MORV in vitro, parallel CPE assays were performed with VSV on normal cells and 19 primary liver cancer cell lines (Table 1). Monolayers of human and murine liver cancer cells were infected with MORV or VSV at MOIs of 1, 0.1, and 0.01. Cell viability was measured 72 hours post-infection. Neither MORV nor VSV induced measurable cell death in ‘normal’ cholangiocytes (H69). However, both MORV and VSV were able to lyse cancer cells and displayed different lytic activities for different types of cancer cell lines (FIG. 3). Two of the 15 cell lines that were tested (HuCCT-1 and PAX-42) were resistant to MORV and VSV, evidenced by 70-100% viability at MOI of 1, 0.1, and 0.01 (FIG. 3). EGI-1 had a similar phenotype of resistance to VSV (MOI of 1) but not to MORV (35% cell viability). Significant virus-induced cancer cell death (<10% viability at MOI of 1) was found in human CCA cell lines SNU-1079, RBE, GBD-1, CAK-1, SNU-245, SNU-308 and SNU-869; in murine CCA cell line SB; in human HCC cell lines HepG2, Hep3B, and Huh7; and in murine HCC cell lines RIL-175 (clones R1LWT and R2LWT), Hepa 1-6, and HCA-1 (FIG. 3). These results indicate that MORV can selectively infect and lyse tumor cells in vitro, while leaving normal cells unaffected.

TABLE 1
Liver Cancer Cell lines.

| Cell line | Site | Media |
|---|---|---|
| CAK-1 | eCCA | RPMI1640 + 10% FBS + AA |
| EGI-1 | eCCA | RPMI1640 + 10% FBS + AA |
| GBD-1 | GBC/eCCA | RPMI1640 + 10% FBS + AA |
| H69 | Normal | DMEM/F12 + 10% FBS +GFs |
| | cholangiocyte | |
| HuCCT1 | iCCA | DMEM + 10% FBS + AA |
| PAX-42 | CCA | DMEM + 10% FBS + AA |
| RBE | iCCA | RPMI1640 + 10% FBS + AA |
| SNU-1079 | iCCA | RPMI1640 + 10% FBS + AA |
| SNU-245 | eCCA | RPMI1640 + 10% FBS + AA |
| SNU-308 | GBC/eCCA | RPMI1640 + 10% FBS + AA |
| SNU-869 | Ampulla of Vater | RPMI1640 + 10% FBS + AA |
| SB | Murine CCA | DMEM + 10% FBS + AA |
| Hep3B | HCC | DMEM + 10% FBS + AA |
| HepG2 | HCC | DMEM + 10% FBS + AA |
| Huh7 | HCC | DMEM + 10% FBS + AA |
| Sk-Hep 1 | HCC | DMEM + 10% FBS + AA |
| RILWT (clone 1 | Murine HCC | DMEM + 10% FBS + AA |
| of RIL-175 cells) | | |
| R2LWT (Clone 2 | Murine HCC | DMEM + 10% FBS + AA |
| of RIL-175 cells) | | |
| HCA-1 | Murine HCC | DMEM + 10% FBS + AA |

Treatment with MORV Did not Induce Production of Type I IFN (IFN-β) in CCA Cells

It was shown that both MORV and VSV are highly sensitive to type I IFN. Because MORV is closely related to VSV, it was investigated if susceptibility in a CCA cell line to MORV infection would correlate with its responsiveness to type I IFN. The ability of MORV and VSV to induce IFN was measured in supernatants of infected cells (MOI of 5) 18 hours post-infection. IFN-β was not detectable in supernatants of cultured CCA cells, which are sensitive to MORV, except in 2 cell lines-GBD-1 (600 ng/mL) and SNU-308 (180 ng/mL) (FIG. 9B). None of the CCA cell lines, including those resistant to VSV, had detectable IFN-β in response to VSV infection. This observation suggests that the resistance to virus-induced CPE in CCA cells does not always correlate with the degree of type I IFN induction. Other virus/host interactions and intrinsic mechanisms specific to tumor cell lines are likely to contribute to oncolysis resistance in the absence of type I IFN secretion and signaling.

### MORV Infects and Efficiently Lyses LDLR and LDLR Knockout Cell Lines

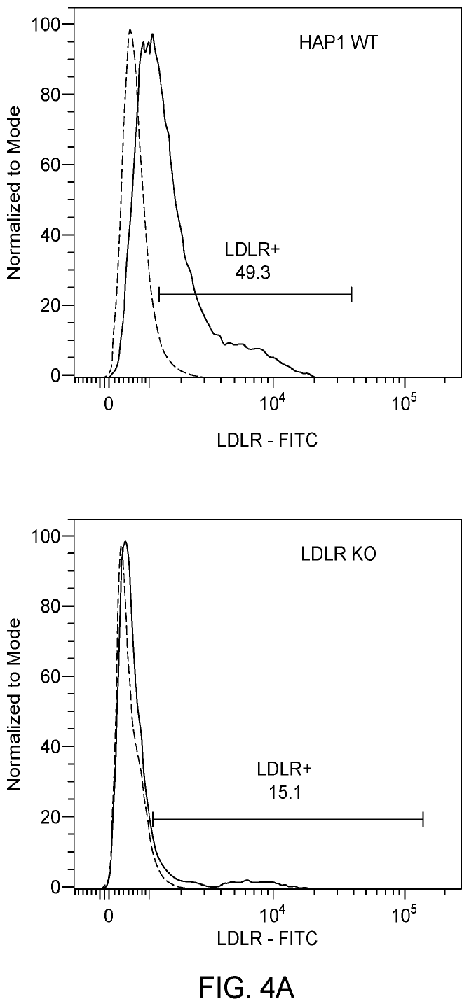

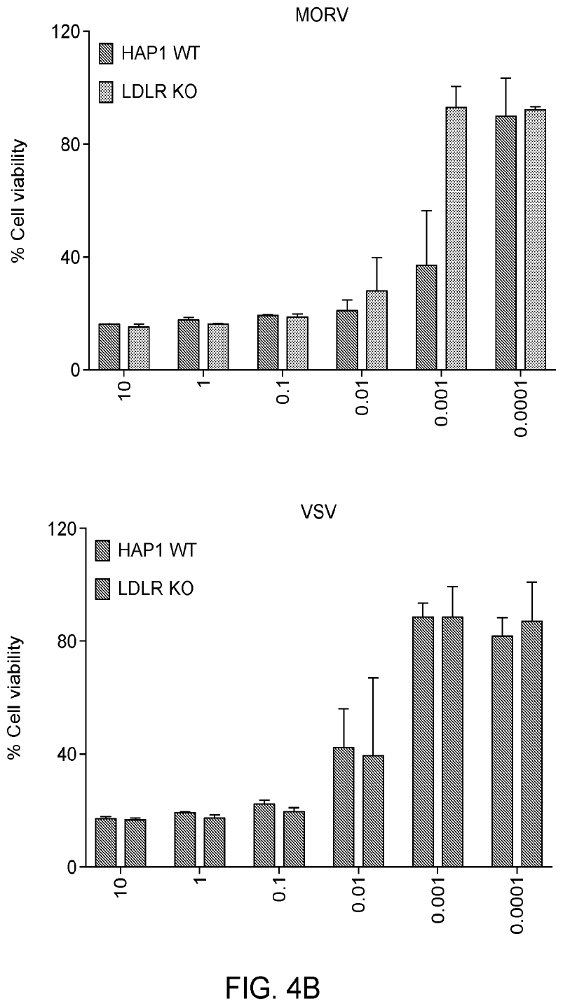

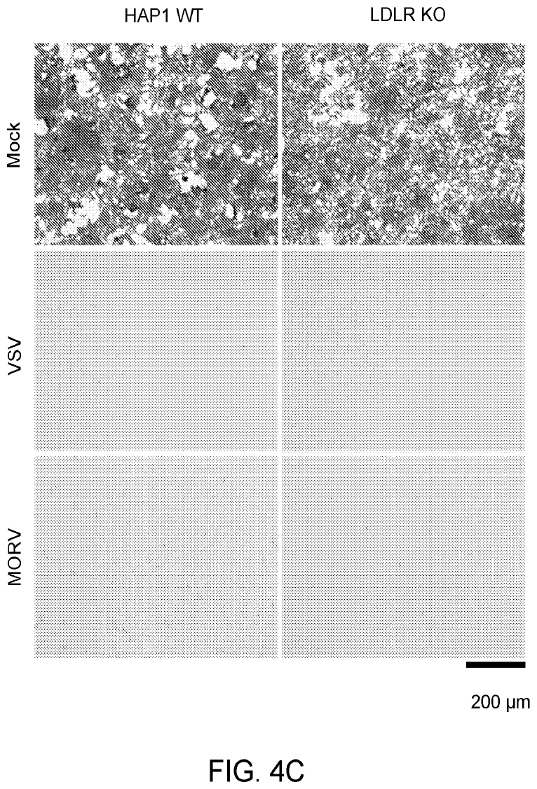

The pantropic nature of VSV implies that it binds and infects host cells via attachment to a cell entry protein that is ubiquitously expressed on the surfaces of mammalian cells. The low-density lipoprotein receptor (LDLR) and its family members have been shown to be putative primary receptors for VSV, but it is unknown whether other Vesiculoviruses such as MORV also use LDLR family members as cellular entry receptors. To address this question, isogenic pairs of wild-type HAP-1 (HAP1 WT) and LDLR knockout (LDLR-KO) cell lines were infected with MORV and VSV across a range of MOIs. The HAP1 cell line (Horizon Discovery) is a human near-haploid cell line derived from KBM-7, a chronic myelogenous leukemia cell line. HAP1 LDLR-KO cells do not express LDLR on their surface. The LDLR gene was disrupted with CRISPR-Cas9 technology to introduce a single nucleotide excision in exon 4 (FIG. 4A). Both MORV and VSV efficiently infected and induced extensive cell lysis in HAP1 WT cells and LDLR-KO cell at indicated MOIs (FIGS. 4B-4C). These observations indicate that cell entry of MORV and VSV is not solely dependent on the availability of LDLR and that these viruses may use different receptors or receptor-independent mechanisms for cell entry.

High-Dose Intranasal Administration of MORV is not Associated with Neurotoxicity or Hepatotoxicity

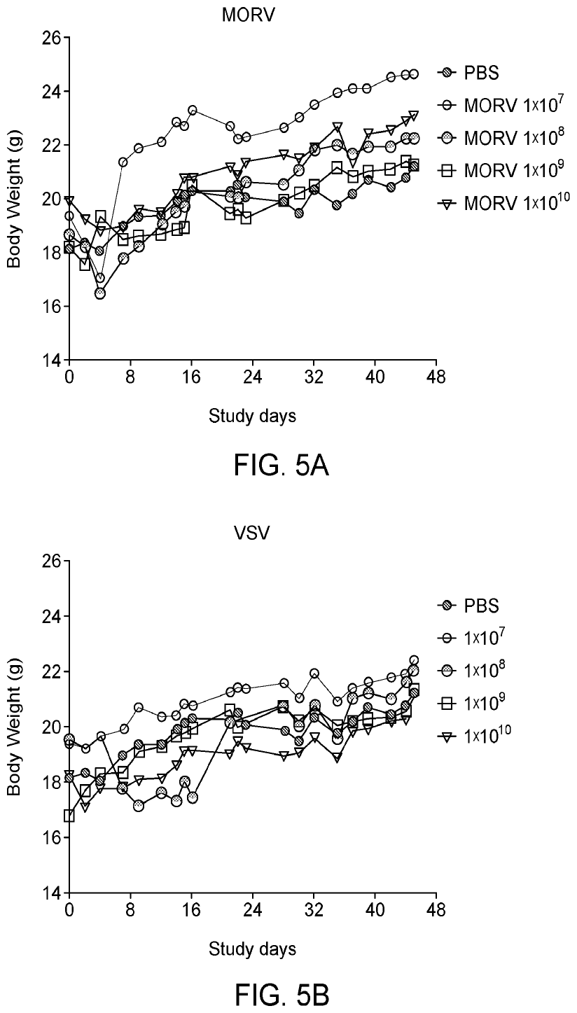

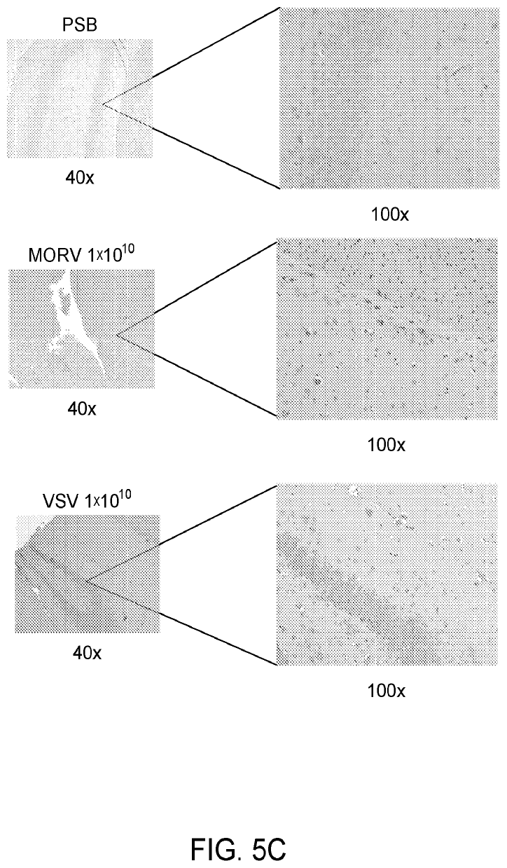

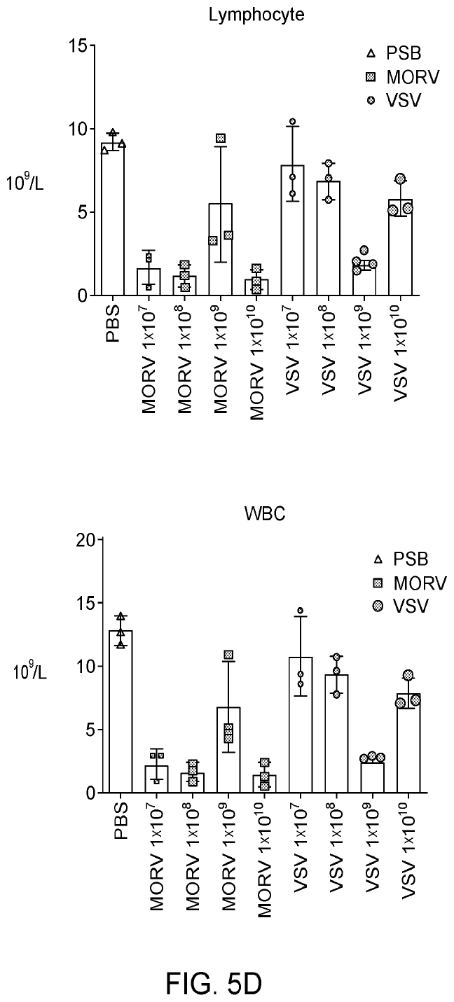

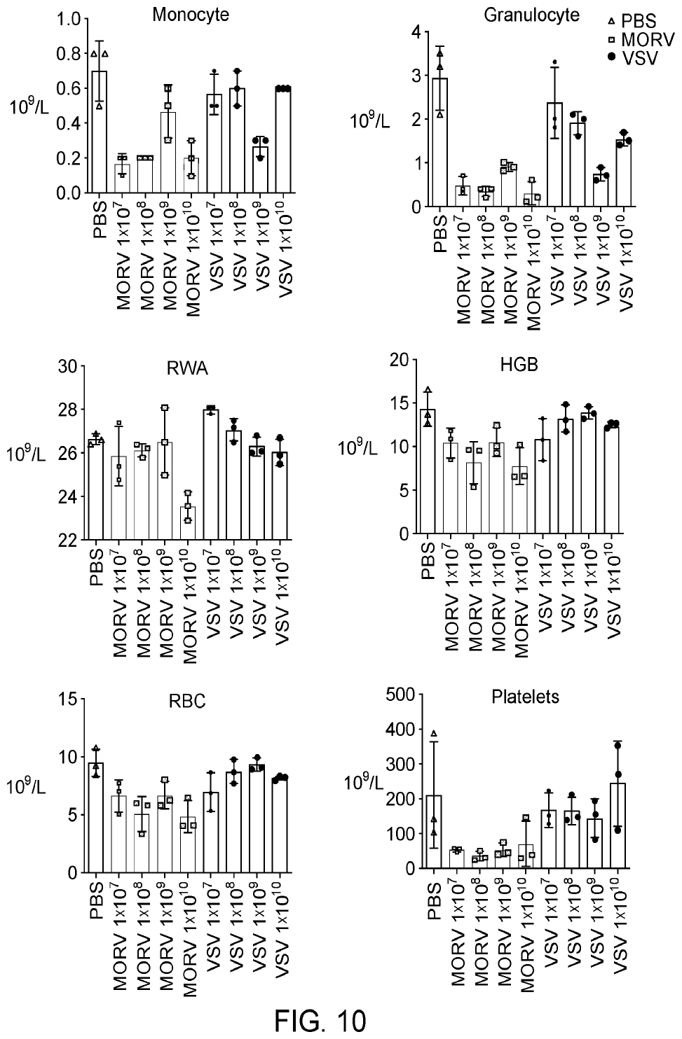

To determine whether MORV is causally associated with brain damage and neurotoxicity in animal models, immunocompetent mice were subjected to 4 intranasal administrations of MORV or VSV across a range of doses (1×10⁷ TCID₅₀/kg, 1×10⁸ TCID₅₀/kg, 1×10⁹ TCID₅₀/kg, and 1×10¹⁰ TCID₅₀/kg). The control group of animals was treated with phosphate-buffered saline (PBS). The highest dose of 1×10¹⁰ TCID₅₀/kg corresponded to 2×10⁸ TCID₅₀ total for a 20 g mouse. Three mice per group were sacrificed 3 days post-infection to assess short-term toxicity and blood, brain, liver, and spleen tissues were collected for further analysis. The remaining animals were monitored for 45 days by a certified veterinarian for signs of toxicity. It was found that the range of low to high doses of MORV and VSV were well tolerated and managed in all groups. In this study, a recombinant VSV rescued from a cDNA clone encoding the VSV Indiana strain, which is known to be significantly attenuated, compared to wild-type VSV was used. Decreases in body weight were not statistically significant 3 days after treatment in the MORV groups; from 7 days after intranasal administration until the end of the study, body weights consistently increased (FIGS. 5A-5B). Consistent with the minimal effects on body weight, a thorough pathology review of brain sections of mice treated with the highest dose of viruses (1×10¹⁰ TCID₅₀/kg) showed that MORV did not cause overt signs of toxicity (FIG. 5C). Analysis of complete blood counts 3 days after treatment with MORV revealed decreases in the numbers of white blood cells, lymphocytes, granulocytes, and platelets (FIG. 5D; FIG. 10). In addition to virus-mediated hematological changes, these changes in blood cell counts might have been caused by hemolysis from the terminal cardiac puncture, which is known to interfere with blood counts and other blood parameters.

### MORV Did Not Extensively Replicate In Non-Tumor-Bearing Immunocompetent Mice

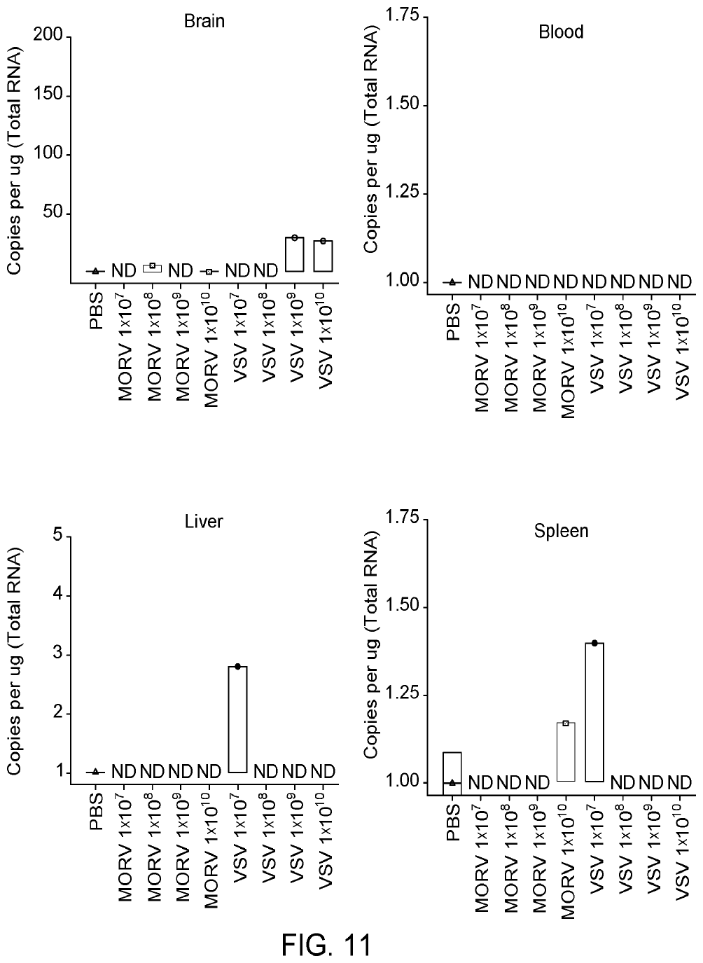

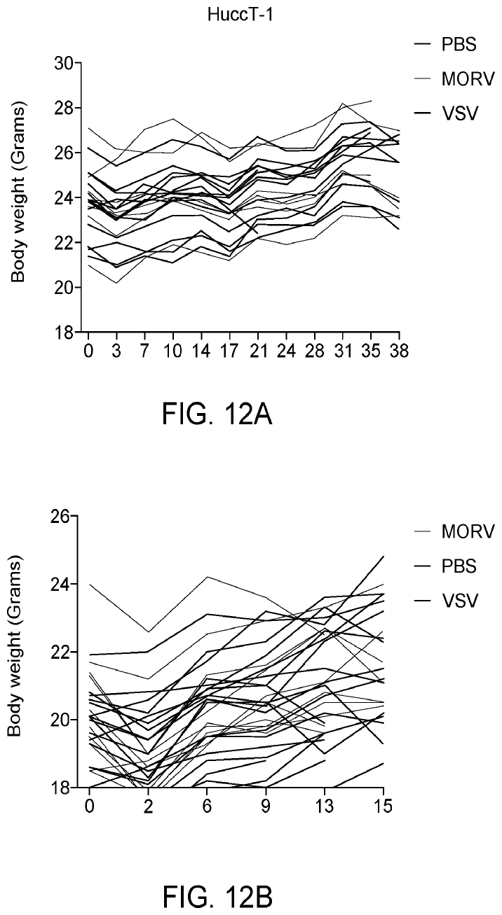

Quantitative real-time polymerase chain reaction (qRT-PCR) was performed to measure MORV-N and VSV-N gene expression in samples prepared from the blood, brain, liver, and spleen tissues of infected mice. MORV-N mRNA was undetectable (<10 copies per μg total RNA) in these tissues from mice treated with MORV doses ranging from 1×10⁷ to 1×10¹⁰ TCID₅₀/kg (FIG. 11). In contrast, ˜30 copies of VSV-N mRNA per g total RNA in brain tissues from mice treated with VSV doses of 1×10⁹ TCID₅₀/kg and 1×10¹⁰ TCID₅₀/kg were detected (FIG. 11). Collectively, these data further indicate that intranasal administration of high doses of MORV (up to 1×10¹⁰ TCID₅₀/kg) did not elicit significant toxicity (100% survival after 45 days) in the treated animals and did not result in physical impairment (Table 2).

TABLE 2
Survival of mice treated with intranasal doses of MORV and VSV
(45 days).

| | 1 × 10⁷ | 1 × 10⁸ | 1 × 10⁹ | 1 × 10¹⁰ |
|---|---|---|---|---|
| Virus | TCID₅₀/kg | TCID₅₀/kg | TCID₅₀/kg | TCID₅₀/kg |
| MORV | 3/3 (100%) | 3/3 (100%) | 3/3 (100%) | 3/3 (100%) |
| VSV | 3/3 (100%) | 3/3 (100%) | 3/3 (100%) | 3/3 (100%) |

### MORV Displayed Heterogeneity In Anti-Tumor Efficacy In Xenograft Models Of Primary Human Liver Cancers

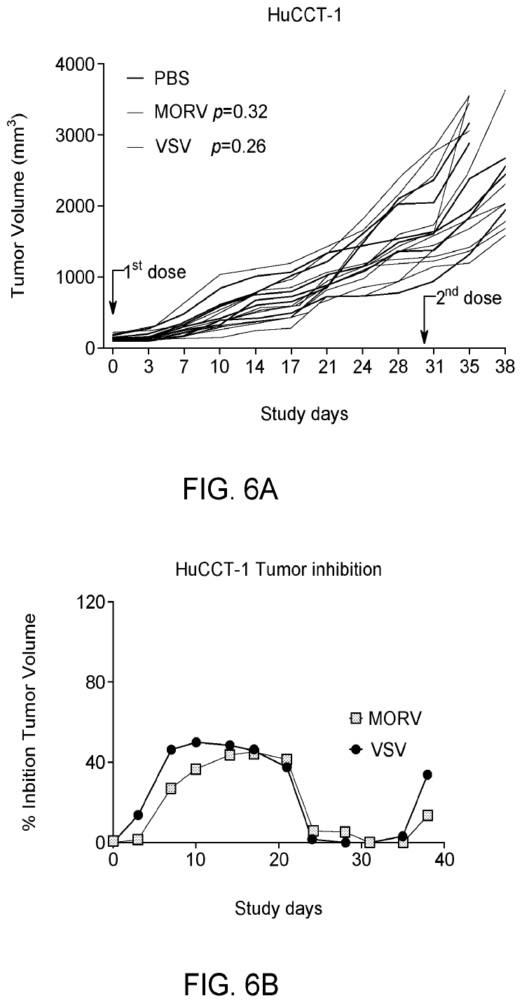

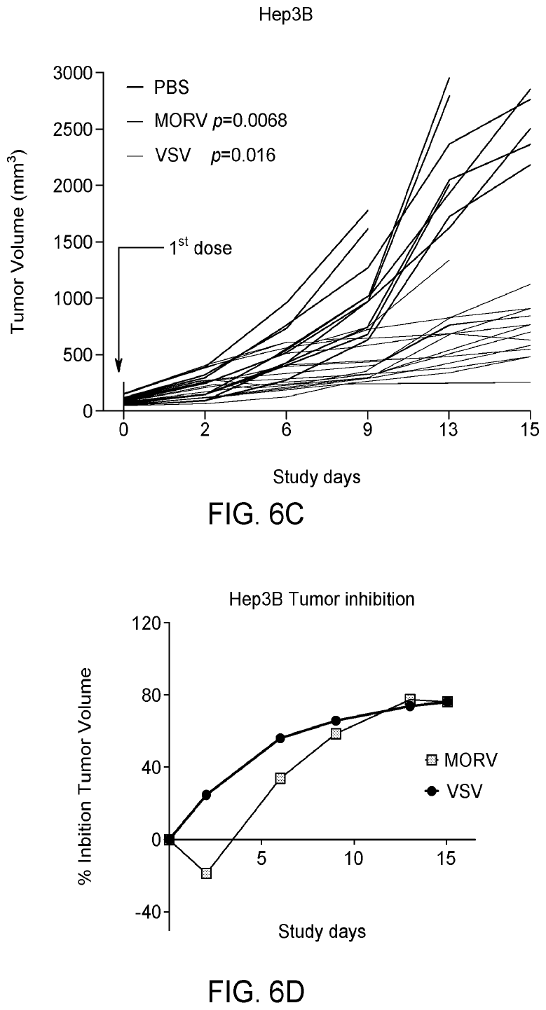

To assess whether tumor cell lines that are resistant or sensitive to MORV-induced death in vitro could display the same phenotype in vivo, resistant (HuCCT-1) and sensitive (Hep3B) human liver cancer cell lines were subcutaneously implanted into female athymic nude (NU/J) mice (n=7) (Jackson Laboratories). After tumors reached a treatable size, intra-tumoral injections (FIG. 6) of MORV or VSV were administered at 1×10⁷ TCID₅₀ units. Body weight, tumor volume and weight, and clinical parameters were recorded 3 times a week. In the human HuCCT-1 CCA model, MORV (p=0.32) and VSV (p=0.26) failed to induce reductions in tumor sizes (FIG. 6A). In contrast, a single injection of MORV (p=0.0068) or VSV (p=0.016) in human Hep3B HCC xenografts resulted in significant reductions in tumor growth (FIG. 6B). No adverse events or virus-related toxicities were observed.

### MORV Efficiently Reduced Tumor Burden In Immune-Competent Mouse Models Of Cholangiocarinoma

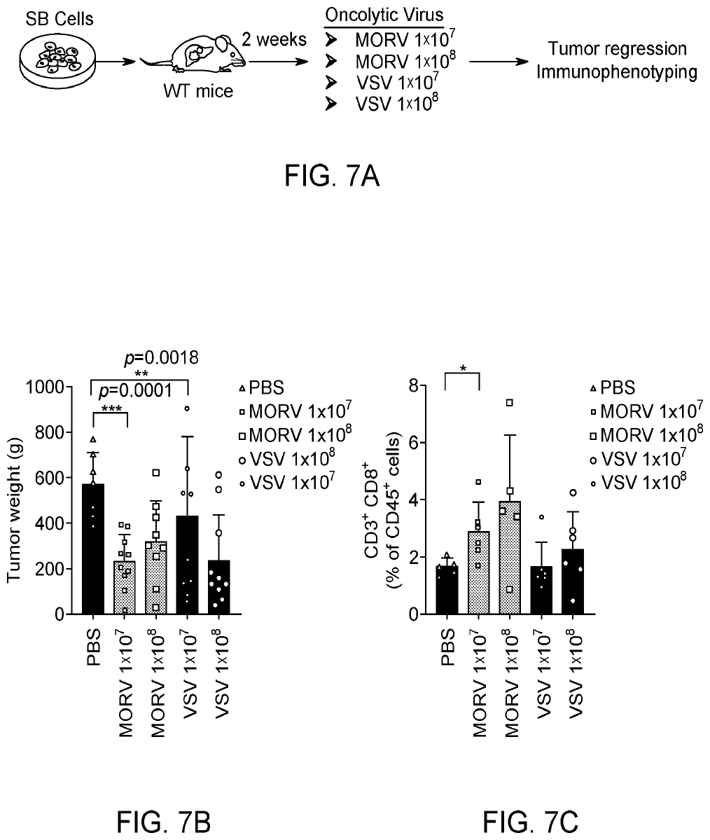

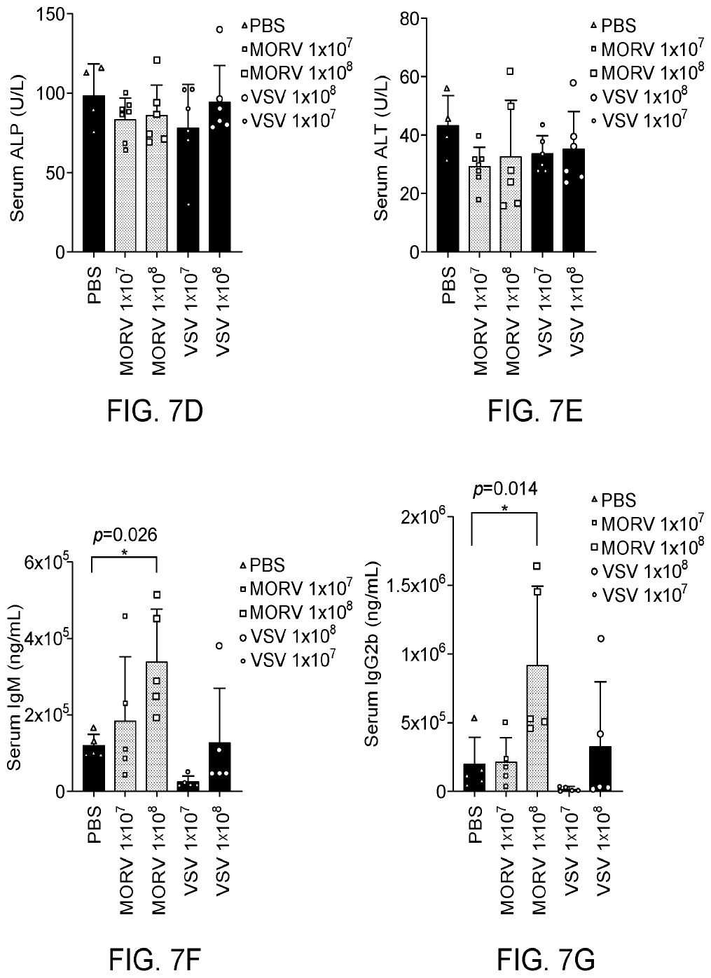

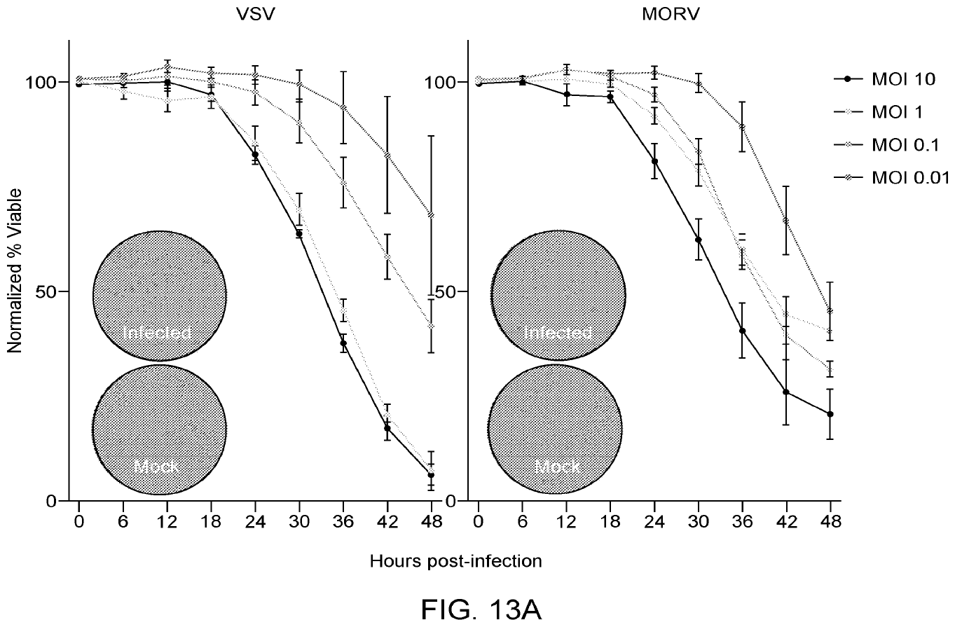

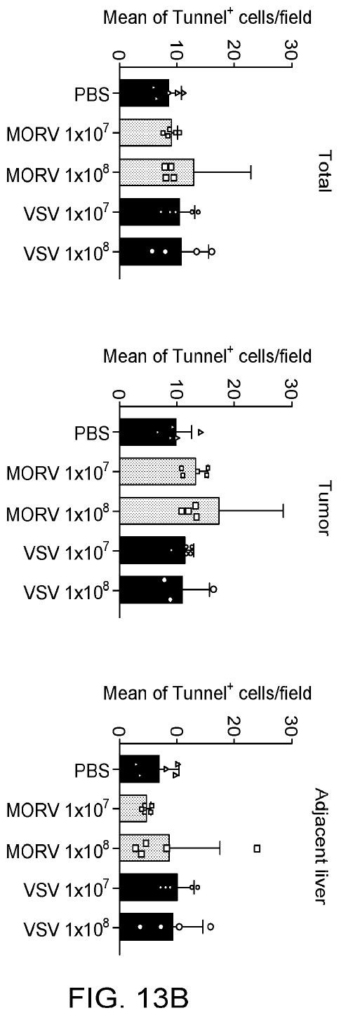

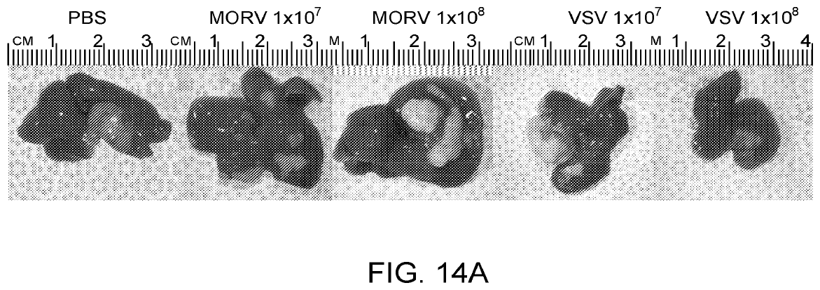

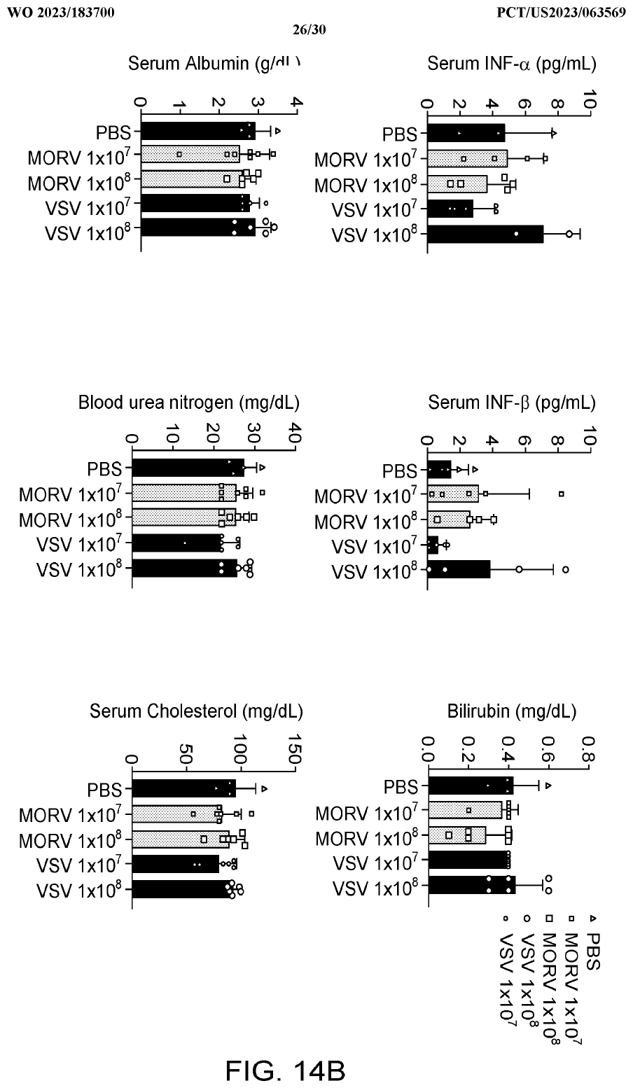

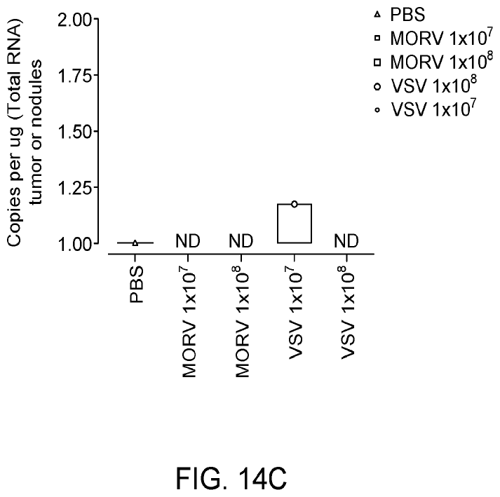

Viral-induced apoptosis was analyzed, and it was confirmed that MORV efficiently infects and kills murine bile duct cancer cells (SB) in vitro (FIG. 13A). To evaluate the cancer-killing properties of MORV particles in vivo, SB cells were surgically implanted into the livers of immunocompetent mice. Fourteen days after orthotopic implantation of SB cells, single intraperitoneal doses of PBS, MORV, or VSV (1×10⁷ TCID₅₀ or 1×10⁸ TCID₅₀) were administered to mice (FIG. 7A). Four weeks after SB cell implantation, mice were sacrificed, and cardiac blood and tumors were collected for downstream analysis. Numbers and sizes of malignant nodules in the livers of MORV-treated mice (p=0.0001) were significantly lower than in VSV-treated mice (p=0.0018) (FIG. 14A). Moreover, tumor regression and disease control in mice treated with a dose of MORV (1×10⁷ TCID₅₀) were identical to those in mice treated with a dose of VSV (1×10⁸ TCID₅₀) (FIG. 7B). Serum levels of alkaline phosphatase (ALP) and alanine aminotransferase (ALT) in mice that were administered MORV and VSV were not significantly different from those of mice that received vehicle (FIGS. 7D-7E). Additionally, TUNEL staining on the adjacent liver tissues showed that treatment did not induce damage to normal (i.e., non-cancer) liver tissues in virus-treated and control mice (FIG. 13B). Furthermore, no significant changes were observed in serum levels of IFN-α, IFN-β, or albumin; blood levels of bilirubin, cholesterol, or urea; and expression levels of MORV-N or VSV-N genes in tumor nodules (FIGS. 14B-14C).

### MORV Altered Antibody Profiles And Immune Cell Components In Tumor Tissues

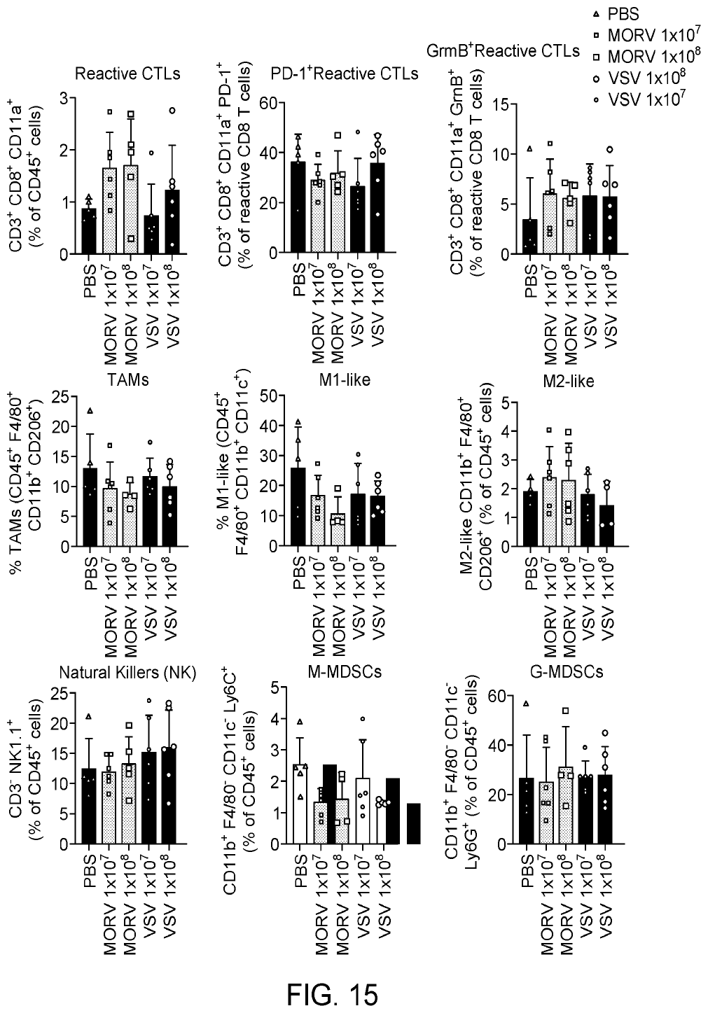

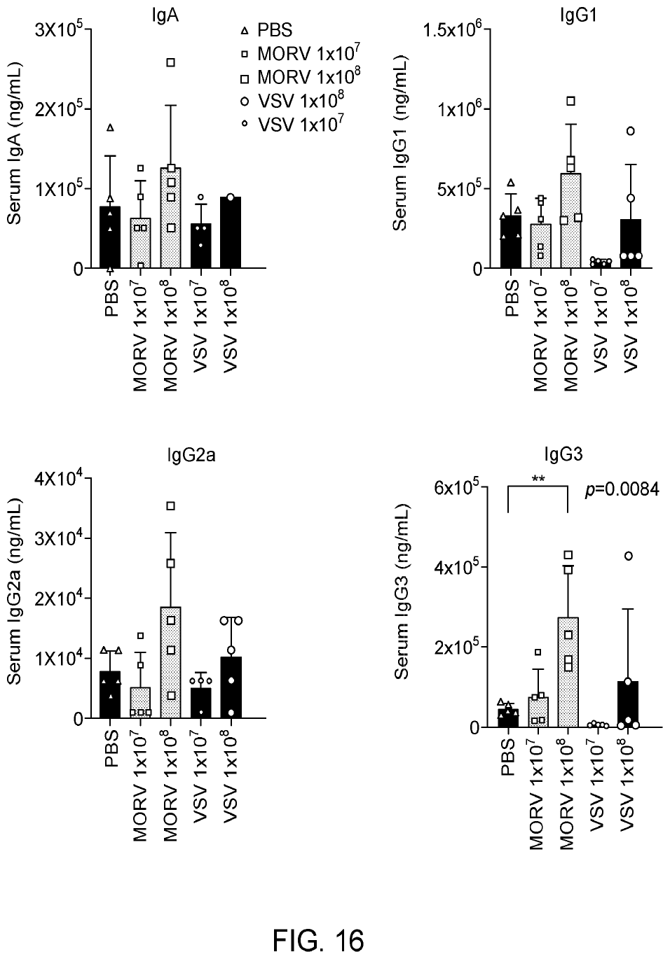

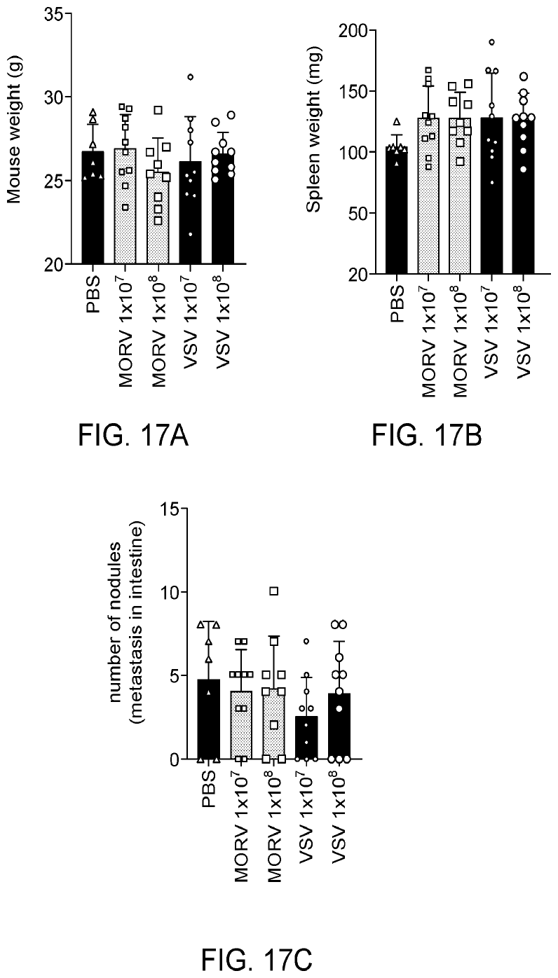

Oncolytic Vesiculoviruses have been shown to exert their antitumor actions by inducing direct cytotoxicity of tumor cells and stimulating host antitumor immune responses. Thus, to understand whether injection of MORV leads to immune responses against tumor cells, immune cell and antibody profiles were assessed in an orthotopic syngeneic model of intrahepatic CCA (SB). Numbers of CD3⁺CD8⁺ cytotoxic T lymphocytes (CTLs) (p=0.029) and amounts of IgG2B (p=0.014), IgG3 (p=0.0084), and IgM (p=0.0256) were significantly greater in MORV-treated mice than in VSV-treated mice. This correlated with the significant reductions in tumor weight (FIGS. 7C, 7F, and 7G; and FIG. 16). Moreover, MORV treatment did not increase the frequency of M2-like tumor-associated macrophages (TAMs), monocytic myeloid-derived suppressor cells (M-MDSCs), or granulocytic (G)-MDSCs (FIG. 15). These results suggest that systemic administration of MORV is associated with an effective and robust antitumor immune response, mainly via CD8⁺T-cell-mediated cytotoxicity. These findings are supported further by the evidence that apoptotic tumor cells are greater in virus-treated mice, especially those treated with MORV, than in the control group (FIG. 13B), with no overt effects on adjacent normal liver cells. As shown in FIG. 14C, MORV-N mRNA was not detected in tumor nodules at the end of the study, suggesting that transient intra-tumoral replication of MORV can induce and sustain durable antitumor immunity. These findings indicate that MORV induces an antitumor immune response mediated by CD8⁺T cell infiltration into the tumor tissue, which will program tumor cells to undergo apoptosis.

Taken together, these results demonstrate that one or more MORVs (e.g., recombinant MORVs) described herein can be administered to a mammal having cancer as a safe and effective oncolytic virotherapy.

### Materials and Methods

### Cell Lines

This study used a panel of 9 human CCA cell lines (HuCCT-1, EGI-1, CAK-1, SNU-245, SNU-308, SNU-869, SNU-1070, RBE, and GBD-1), a carcinoma of the ampulla of Vater, a transformed cholangiocyte cell line (H69), a murine CCA cell line (SB), 4 HCC cell lines (HepG2, Hep3B, Huh7, and Sk-Hep-1) and 3 murine HCC cell lines (R1LWT, R2LWT, and HCA-1). All cell lines were cultured at 37° C. with 5% CO₂ in medium supplemented with L-glutamine and antibiotics (100 g/mL penicillin and 100 g/mL streptomycin). EGI-1, CAK-1, SNU-245, SNU-308, SNU-869, SNU-1070, RBE, and GBD-1 were maintained in RPMI 1640 medium with 10% fetal bovine serum (FBS). PAX-42, HuCCT-1, H69, BHK-21 (baby hamster kidney fibroblast), A549 (human lung tumor cells), and Vero (African green monkey kidney) were maintained in Dulbecco's modified Eagle's medium (DMEM) with 10% FBS.

HAP1 parental cell line and LDLR-KO cell line were obtained from Horizon and maintained in Iscove's modified Dulbecco's medium (IMDM) supplemented with 10% FBS and 1% antibiotic. The HuCCT-1 cell line was obtained from the Japanese Collection of Research Bioresources (JCRB). EGI-1 was purchased from the German Collection of Microorganisms and Cell Cultures (DSMZ). SNU-245, SNU-308, SNU-869, and SNU-1079 were obtained from the Korean cell line bank (KCLB). RBE was purchased from the National Bio-Resource Project of the MEXT, Japan (RIKEN). BHK-21, A549, and Vero cells were obtained from the American Type Culture Collection (ATCC; Manassas, VA). Hep3B, HepG2, Huh7, Sk-Hep1, and Hepa 1-6 were purchased from the ATCC and were grown in DMEM with 10% FBS and in RPMI with 10% FBS, respectively. HCA-1, and SB cells, and two clones derived from the RIL-175 cell line (R1LWT, R2LWT) were grown in DMEM with 10% FBS.

### Oncolytic Viruses

MORV was obtained from the University of Texas Medical Branch (UTMB) World Reference Center for Emerging Viruses and Arboviruses (WRCEVA). A laboratory-adapted viral clone of MORV was generated with sequential plaque purifications on Vero cells (ATCC). RNA-sequencing was applied to confirm the full-length MORV genome. The full-length MORV genome (11,181 nucleotides) comprised of genes encoding the nucleoprotein (MORV-N), phosphoprotein (MORV-P), matrix protein (MORV-M), glycoprotein (MORV-G), and RNA-directed RNA polymerase L protein (MORV-L), was synthesized (Genscript) from the laboratory-adapted viral clone of MORV and was subcloned into plasmid (pMORV-XN2). pMORV-XN2, along with helper plasmids (pMORV-P, pMORV-N, and pMORV-L), are as described elsewhere (Faul et al., Viruses, 1:832-851 (2009)). These plasmids were used to express the antigenomic-sense RNA of MORV under the bacteriophage T7 promoter to generate recombinant MORV. However, in this study, wild-type MORV rather than recombinant MORV was used. VSV was rescued using BHK-21 cells from a plasmid (pVSV-XN2) containing the VSV Indiana genome serotype. All viruses were rescued with a vaccinia rescue system and were propagated and titrated on BHK-21 cells, as previously described. Sucrose density gradient centrifugation was used to obtain purified viral particles (VSV, MORV, and recombinant MORV) before in vitro and in vivo studies.

### Evolutionary Analysis Of MORV Glycoprotein (MORV-G) Amino Acid Sequence By Maximum Likelihood Method

All viral glycoprotein sequences were retrieved from UniProt (uniprot.org). The evolutionary history was inferred by using the maximum likelihood method and JTT matrix-based model. The tree with the highest log likelihood is shown (FIG. 8A). The percentage of trees in which the associated taxa clustered together is shown next to the branches. Initial tree(s) for the heuristic search were obtained automatically by applying Neighbor-Join and BioNJ algorithms to a matrix of pairwise distances estimated with the JTT model and then selecting the topology with superior log likelihood value. The tree is drawn to scale, with branch lengths measured in the number of substitutions per site. An evolutionary analysis was conducted in MEGA X 5, using 15 amino acid sequences comprising 574 positions in the final dataset.

### Transmission Electron Microscopy

MORV was propagated in BHK-21 cells. Supernatants were harvested approximately 48 hours post-infection and clarified by centrifugation at 1,500 g in a benchtop centrifuge for 20 minutes. MORV-containing supernatants were layered on top of a 1-step sucrose gradient (60% (w/v) layered on top of 20% (w/v)) and centrifuged at 100,000 g for 2 hours. The visible virus band was harvested from between the sucrose layers by pipetting from the meniscus. MORV was diluted in buffer (20 mM Tris, 150 mM NaCl; pH 7.8) and centrifuged at 100,000 g for 1.5 hours. The supernatant was discarded and the virus pellet was resuspended in 100 μL of buffer (20 mM Tris, 150 mM NaCl; pH 7.8). 2 μL of this sample was applied to R2/2 UltrAUfoil grids and blotted manually before plunge freezing in liquid ethane. The vitrified specimen was imaged with a FEI Titan Krios transmission electron microscope (ThermoFisher) operating at an accelerating voltage of 300 keV with a Gatan K2 Summit direct electron detector camera.

### Viral Plaque Formation Assays

Serial dilutions (1:100 to 1:10⁸) ofMORV and VSV were used to infect Vero cells (5×10⁵) in tissue culture dishes (60 mm) for 1 hour. Cells were then washed with PBS and overlaid with 1.5% bacteriological agar (Sigma). Forty-eight hours post-infection, infected cells were stained with crystal violet, and plaques were counted to determine the plaque-forming unit per ml (PFU/mL). Visualization of MORV and VSV plaques (1:10⁵ dilution) after crystal violet staining was performed with an Invitrogen EVOS® FL Auto Imaging System.

### IFN Sensitivity Assays

IFN-sensitive lung cancer cells (A549) were seeded in a 96-well plate at a density of 2×10⁴ cells/well and cultured overnight. Twenty-four hours post-infection, cells were pretreated with different concentrations of universal type I IFN-α (Catalog No. 11105-1; PBL Assay Science) added directly into the culture medium. After overnight incubation, fresh medium containing universal Type I IFN-α was added to the cells, and the cells were infected with MORV or VSV at a MOI of 0.01. Cell viability was assessed with a Cell Titer 96™ AQueous One Solution Cell Proliferation Assay (Promega). Absorbance measurements at 490 nm were normalized to the maximum read per cell line, representing 100% viability. Data are shown from 3 independent experiments. For all cell viability experiments, absorbance was read with a Cytation™ 3 plate reader (BioTeK). Data are expressed as means of triplicates from three independent experiments SEM.

### Type IIFN-f Production Assays

Cells were plated (2×10⁴ cells per well) in 96-well plates and infected with MORV or VSV at MOI of 5. Cell supernatants were harvested 18 hours post-infection, and IFN-β levels were measured with a VeriKine-HS™ Human IFN-β TCM ELISA kit (Catalog No. 41435-1; PBL Assay Science), as recommended by the manufacturer.

### Virus Neutralization Assays

MORV or VSV (400 TCID₅₀) was incubated for 1 hour (37° C., 5% CO₂) in 96-well plates with a specific polyclonal rabbit antibody against VSV-G, VSV-N, or VSV-M (Imanis) at increasing dilutions (1:10, 1:20, 1:40; and 1:10,240 final antibody dilution) of serum from patients (PT13-D22 and PT14-22) treated with VSV-INF-β in the context of a clinical study (ClinicalTrials.gov: NCT01628640); or normal human serum. This incubation was followed by addition of Vero cells (2×10⁴) directly into each well. After 24-hour incubation, cell viability was measured with an MTS assay (Promega), as indicated above. Data are expressed as means of triplicates from three independent experiments±SEM.

### Cell Viability Assays

For all cytotoxicity assays (96-well format), cells (1.5×10⁴) were infected with MORV or VSV at MOI of 1, 0.1, and 0.01 in serum-free GIBCO™ Minimum Essential Media (Opti-MEM). Cell viability was determined with Cell Titer 96™ AQueous One Solution Cell Proliferation Assay. Data were generated as means of six replicates from six independent experiments, ±SEM.

### Visualization Of Virus-Induced Cytopathic Effects In Cholangiocarcinoma Cells

MORV and VSV were used to infect adherent cells (95×10⁵ cells per well) in 6-well plates at MOI of 0.1. Cells were incubated at 37° C. until analysis. At 72 hours after infection, cells were fixed with 5% glutaraldehyde and stained with 0.1% crystal violet to visualize cellular morphology and remaining adherence, which indicates cell viability. Pictures of representative areas were taken.

### Real-Time Analysis of Virus Induced Apoptosis

Murine cholangiocarcinoma (SB) cells (1×10⁴ cells per well) were plated in 96-well plates and rested overnight. The next day, serial dilutions of virus, starting at MOT of 10, were added to wells in triplicate. Annexin V Red (Essen Bioscience) was added to each well, including control wells with no virus. Plates were imaged every 6 hours for 48 hours in the IncuCyte S3 system (Essen Bioscience), recording both phase and red fluorescence images. Total red area for each well was quantified and normalized for the entire experiment. Control wells were used to subtract background and as representative of 100% viability. Data were plotted as mean±SEM. Representative images are included from the 48-hour timepoint, with either virus-treated (MOI of 10) or control wells (mock-infected).

### Flow Cytometry

For flow cytometry, cells of the HAP1 parental cell line (WT) and LDLR-KO cells (1×10⁶ cells per sample) were resuspended in PBS, plated in 96-well format, and washed twice at 350 g for 3 minutes. This step was followed by incubation with 3% bovine serum albumin for 30 minutes at room temperature to block nonspecific staining. Cells were washed, and incubated with primary antibody (LDLR antibody, 10785-1-AP; Proteintech) (0.2 μg per test) for 2 hours at room temperature. Cells then were washed twice with 2% FBS in PBS. Secondary antibody (Goat anti-rabbit IgG-FITC, #sc-2012; Santa Cruz) was added to the cells at 1:400 and the cells were incubated for 1 hour at room temperature. Cells were washed twice with 2% FBS in PBS and resuspended in 2% formalin before being acquired with an LSRFortessa (BD). Data were collected in the FITC and scatter channels and were analyzed in FlowJo® version 10.7.1 (BD). Isotype control samples were used to set positive gates (at 5% on isotype samples) that were applied to full-stained samples.

### Susceptibility And Effects Of MORV And VSV In An LDLR-KO Cell Line (HAP-1)

HAP1 (Horizon Discovery) is a near-haploid human cell line derived from chronic myelogenous leukemia cell line KBM-7. HAP1 knockout (HAP1-KO) cells have a single base pair deletion in exon 4 of the LDLR gene (constructed with CRISPR-Cas9 technology). HAP1 WT cells or LDLR-KO cells (1×10⁴ cells per well) were plated in 96-well plates and rested overnight. The following day, cells were infected with serial dilutions of the indicated virus, starting with MOI of 10. After 72 hours, cell viability was measured with MTS assays, as described above. Data are shown as mean±SEM from three independent experiments.

### Animal Studies

The in vivo evaluations described below were conducted.

Toxicology And Biodistribution Of Virus Administrated Via The Nasal Route. To determine whether treatment with MORV and VSV could be associated with neurotoxicity, female C57BL/6J mice (N=78; n=6 mice per group), including controls, were intranasally administered (10 μL per nostril) PBS or a dose (1×10⁷, 1×10⁸, 1×10⁹, or 1×10¹⁰ TCID₅₀/kg) of MORV or VSV. Body weight, temperature, behavior, and clinical signs were monitored by a board-certified veterinarian at least 3 times per week to detect signs of toxicity. Three days post-infection, three mice per group were sacrificed and tissues (brain, liver, and spleen) were collected for evaluation of short-term toxicity and viral biodistribution. The remaining mice were monitored for 45 days, and body weights and clinical observations were recorded at least three times per week for the study duration.

Blood Tests. Blood was collected from the submandibular vein (cheek bleed) on day 3 and from cardiac puncture on day 45. Blood was collected for complete blood counts in BD Microtainer® tubes with ethylenediaminetetraacetic acid or lithium heparin (Becton, Dickinson and Company) and for serum analysis in BD Microtainer® SST tubes (Becton, Dickinson, and Company). Analysis of complete blood counts was performed in a Piccolo Xpress® chemistry analyzer (Abaxis), and blood chemistry analysis was done in a VetScan® HM5 Hematology Analyzer (Abaxis).

Viral RNA Extraction And Quantitative RT-PCR. At 72 hours post-infection, female C57BL/6J mice (N=39; n=3 mice per group) from PBS-treated, MORV-infected, and VSV-infected groups were sacrificed. RNA was extracted from tissues and blood (RNeasy Plus Universal Mini Kit, Cat. 73404; QlAamp Viral RNA, Cat. 52904; Qiagen). MORV N and VSV N mRNA in brain, liver, spleen, and blood was quantified with 1-step multiplex qRT-PCR (LightCycler® 480 RNA Master Hydrolysis; Roche Diagnostics), as recommended by the manufacturer. All qRT-PCR assays, including detection of viral gene expression in the tumor (SB in vivo model) were conducted on a LightCycler® 480 II with the following primers and probes: VSV-N Forward primer, 5′-TGATAGTACCGGAGGATTGACGAC-3′ (SEQ ID NO:7); VSV-N Reverse primer, 5′-CTGCATCATATCAGGAGTCGGT-3′ (SEQ ID NO:8); VSV-N (Probe), 5′-FAM-TCGACCACATCTCTGCCTTGTGGCGGTGCA-ZEN/IBFQ-3′ (SEQ ID NO:9); MORV-N Forward primer, 5′-CCCCAATGCAGGGGGACTCACAAC-3′ (SEQ ID NO:10); MORV-N Reverse primer, 5′-TAGCAACATGTCTGGGGTGGGC-3′ (SEQ ID NO:11); MORV-N (Probe), 5′-HEX-TCTACAACATCTCGTCCTTGAGGAGGAGCA-ZEN/IBFQ-3′ (SEQ ID NO:12); MORV-GForward primer, 5′-TCGCAGGACCCATCATTCCTC-3′(SEQ ID NO:13); MORV-G Reverse primer, 5′-CAACATCTTCGTAGGGGTAC-3′ (SEQ ID NO:14); MORV-G (Probe), 5′-Cy5-CGGTCGTGGTTCCGCTGATTACTCCTCTCA-TAO/IBRQ-3′ (SEQ ID NO:15).

In Vivo Efficacy Study In Human CCA And HCC Xenograft Models. To evaluate the in vivo therapeutic efficacy of oncolytic viruses MORV and VSV in subcutaneous xenograft models of human liver bile duct carcinoma and hepatocellular carcinoma, HuCCTI (CCA) and Hep3B (HCC) cells (2×10⁶) were subcutaneously inoculated into the right flanks of female athymic nude (NU/J) mice (n=7 mice per group) (Jackson Laboratories). When tumors reached an average size of 100-200 mm³, mice were randomized into treatment groups and were dosed within 24 hours of randomization. Each mouse received 2 (HuCCT-1) or 1 (Hep3B) intra-tumoral injections. Mice in the HuCCT-1 cohort received 2 doses 1 week apart of 50 μL containing PBS or 1×10⁷ TCID₅₀ units of MORV or VSV). Mice in the Hep3B cohort received a single dose of PBS or 1×10⁷ TCID₅₀ units of MORV or VSV. Tumor volume and body weight were monitored. Mice were euthanized when adverse effects were observed or when tumor size was larger than 2,000 mm³. Tumor volume was calculated with the following equation: (longest diameter*shortest diameter²)/2. Tumor images were taken before resection and tumor weights recorded after resection.

In Vivo Efficacy In A Syngeneic Orthotopic Mouse Model Of Cholangiocarcinoma (CCA). Female C57BL/6J mice (N=50; n=10 mice per group) were purchased from Jackson Laboratories. Mice were anesthetized with 1.5-3% isoflurane. Under deep anesthesia, the abdominal cavity was opened with a 1-cm incision below the xiphoid process. A sterile cotton-tipped applicator was used to expose the superolateral aspect of the medial lobe of the liver. With a 27-gauge needle, 20 μL of standard medium containing 0.75×10⁶ SB cells (murine CCA cells) was injected into the lateral aspect of the medial lobe. A cotton-tipped applicator was held over the injection site to prevent cell leakage and blood loss. Subsequently, the abdominal wall and skin were closed in separate layers with absorbable chromic 3-0 gut and nylon 4-0 skin suture material. All virus injections were initiated 7 days after implantation of SB cells. For experiments that used oncolytic viruses, mice were randomly assigned to vehicle (PBS), MORV 1×10⁷ TCID₅₀, MORV 1×10⁸ TCID₅₀, VSV 1×10⁷ TCID₅₀, or VSV 1×10⁸ TCID₅₀. Viral preparations were administered in 50-μL single intraperitoneal injections. Animals were monitored daily for 1 week and then weekly for any changes in appearance or behavior, including any signs of morbidity or mortality. Four weeks after implantation of SB cells, mice were sacrificed. Tumor, adjacent liver, spleen, and blood were collected for downstream analysis. Images of resected tumors were taken at study termination.

Analysis Of Tumor-Infiltrating Immune Cells. Upon excision, tumors from five mice per group were dissociated with gentleMACS™ Octo Dissociator (Miltenyi), according to the manufacturer's protocol. CD45⁺ cells were isolated with CD45 (TIL) mouse microbeads (Miltenyi). Cells were incubated with Fixable Viability Stain 510 (BD Horizon™) for 15 minutes, followed by anti-Fc blocking reagent (Miltenyi) for 10 minutes prior to surface staining. Cells were stained, followed by data acquisition on a MACSQuant Analyzer 10 optical bench flow cytometer (Miltenyi). All antibodies were used according to the manufacturer's recommendation. Fluorescence Minus One control was used for each independent experiment to establish gating. For intracellular staining of granzyme B, cells were stained with the intracellular staining kit (Miltenyi). Analysis was performed with FlowJo® (TreeStar). Forward scatter and side scatter were used to exclude cell debris and doublets.

Flow Cytometry Analysis Antibodies. The following antibodies were used for flow cytometry staining: F4/80-PE (REA126; Miltenyi), CD11b-PE-Cy5 (M1/70; eBioscience), CD206-PE-Cy7 (C068C2; BioLegend), F4/80-PE-Vio770 (REA126; Miltenyi), CD11c-APC (REA754; Miltenyi), Ly6G-PE (Rat 1A8; Miltenyi), Ly6C-APC-Vio770 (REA796; Miltenyi) CD3-APC-Vio770 (REA641; Miltenyi), CD8-BV421 (53-6.7; BD Horizon), CD11a-PE-Vio770 (REA880; Miltenyi), PD-1-PerCP-Vio700 (REA802; Miltenyi), and granzyme B-PE (REA226; Miltenyi).

Murine Immunoglobulin Isotyping After Treatment With MORV And VSV For analysis of antibody production, serum samples from five mice per group were obtained from whole blood collected in BD Microtainer® tubes. Serum immunoglobulin subclass antibody responses (IgG1, IgGA, IgG2a, IgG2b, IgG3, and IgM) were determined after infection with MORV or VSV by using mouse isotyping multiplex assay (MGAMMAG-300K; Millipore Sigma), as recommended by the manufacturer.

Serum Cytokines. Serum levels of IFN-α and IFN-β were measured with IFN-α and IFN-β-Plex Mouse Panel (EPX02A-22187-901; ThermoFischer). Blood chemistry analysis (serum albumin, blood urea nitrogen, and serum cholesterol) was performed in a Piccolo Xpress® chemistry analyzer (Abaxis).

Histopathological Analysis. Assessment of any abnormal changes in brain, liver, or spleen was determined by histopathological evaluation of H&E-stained images, reviewed by a board-certified pathologist. HALO v3.1.1076.379 was used to measure the percentage tumor necrotic area.

TUNEL Assay Immunohistochemistry. Liver histology was examined with tissue fixed in 10% formalin, dehydrated, and embedded in paraffin. Terminal deoxynucleotidyl transferase deoxyuridine triphosphate nick-end labeling (TUNEL) assays were carried out with the ApopTag® Peroxidase In Situ Apoptosis Detection Kit (Millipore Sigma). Diaminobenzidine was used as a peroxidase substrate (Vector Laboratories). 0.5% methyl green was used for the counterstain. Positive cells were identified in five representative fields of adjacent liver and five usual fields in tumor for each group.

### Statistical Analysis

All values were expressed as mean±standard deviation, and the results were analyzed by one-way analysis of variance, followed by the Tukey test for multiple comparisons and the Kaplan-Meier method for survival, using statistical software in GraphPad Prism®, version 8 (GraphPad Software). A p value less than 0.05 was considered significant.

### Example 2: Engineering of an Attenuated Strain of Morreton Virus (MORV)

This Example describes the generation of an attenuated MORV strain.

Laboratory based attenuated of MORV

MORV was obtained from the UTMB WRCEVA. A laboratory-adapted viral clone of MORV was generated using sequential plaque purifications (˜20 passages) on human cancer cells (A549, Hela) and Vero cells (ATCC, Manassas, VA). RNA-sequencing was applied to confirm the full-length MORV genome which differs in its intergenic region from the wild-type MORV from Genbank (NC_034508.1).

Recombinant MORV genome

The full-length MORV genome (11,181 nucleotides) comprising genes encoding for the nucleoprotein (MORV-N), phosphoprotein (MORV-P), matrix protein (MORV-M), glycoprotein (MORV-G), and RNA-directed RNA polymerase L protein (MORV-L), was codon optimized for expression in mammalian cells and synthesized (Genscript, USA) from the laboratory-adapted viral clone of MORV. A chimeric leader sequence from the VSV Indiana strain and MORV and a synthetic intergenic region to enable proper translation of viral proteins were added. The resultant full-length MORV genome was subcloned into plasmid (pMORV-XN2). pMORV-XN2, along with helper plasmids (pMORV-P, pMORV-N, and pMORV-L as described elsewhere; Faul et al., Viruses, 1:832-851 (2009)), were used to express the antigenomic-sense RNA of MORV under the bacteriophage T7 promoter. The generated recombinant MORV (rMORV) was recovered using a vaccinia rescue system, and the recovered rMORV was propagated and titrated on BHK-21 cells. Sucrose density gradient centrifugation was used to obtain purified viral particles (VSV, MORV and recombinant MORV) before in vitro and in vivo studies.

Intergenic sequences added before the start and stop codon of each gene of MORV:
5′-AACAGnnATC (antiparallel of the start sequence; SEQ ID NO: 1)-
ATG-target gene-STOP-3′ 5′-TATGAAAAAAA (antiparallel of the end
sequence; SEQ ID NO: 2)-3′
Chimeric leader sequence (before the N gene of MORV):
(SEQ ID NO: 3)
5′-AGACAAACAAACCATTATTACAATTAAAAGGCTCAGGAGAACCTTCAACAGCAATCGAA-3′
Laboratory strain of MORV virus (RNA-seq from sucrose gradient purified particles)
SEQ ID NO: 5
AGACAAACAAACCATTATTACAATTAAAAGGCTCAGGAGAACCTTCAACAGCAATCGAAATGTCGGTT
ACAGTCAAAAGAATCATTGACAACTCTGTCATCCTCCCCAAATTGCCAGCCAATGAGGATCCAGTTGA
ATATCCGGGTGACTATTTCAAAAAGACAAATGAGGTCCCTGTGTACATCAACACCAGCAAGACTCTAA
ATGATCTGAGAGGGTATGTTTATCAAGGACTAAAAACAGGCAATGTGTCTATAATCCATGTCAACAGC
TATCTGTATGCAGCATTAAGAGACATTAAAGGAAAACTCGACAAAGATTGGATCAGCTTCGGAGTTCA
GATCGGGAAAACCGGAGATGAAGTTGGAATATTCAATTTAGTGTCAGTCAAAACTCTGGAAGGAATTA
TTCCAGATGGAGTATCAGATGCATCAAGAACTAGCGCAGATGATGCCTGGCTTCCCCTTTATTTACTG
GGGCTTTACAGAGTGGGGAGGACTCAAATGCCAGAATACCGGAAGAAATTGATGGATGGTCTAATTAA
TCAGTGCAAAATGATCAATGAGAAGTTCGAACCTCTAGTTCCTGAGGGGCGTGACATCTTTGATGTGT
GGGGAAACGACAGCAATTATACCAAAATCGTAGCCGCCGTCGACATGTTCTTTCATATGTTCAAAAAG
CATGAGAAGGCCTCTTTCAGATATGGAACTATAGTGTCCAGGTTCAAGGACTGCGCCGCATTGGCGAC
ATTTGGACATCTATGTAAAATCACTGGCATGTCTACAGAAGATGTGACCACTTGGATCCTCAATAGAG
AAGTGGCAGATGAAATGGTTCAAATGATGCTTCCAGGCCAAGAAATTGATAAGGCGGATTCTTACATG
CCTTACCTCATAGACTTCGGACTGTCTTCAAAGTCTCCATACTCGTCCGTAAAAAACCCGGCTTTCCA
CTTTTGGGGTCAATTGACTGCATTGCTGTTGAGATCAACTCGAGCAAGGAATGCCAGACAACCTGACG
ACATCGAGTATACATCATTGACAACAGCAGGTCTCCTATATGCTTATGCAGTAGGTTCATCAGCAGAC
CTTGCGCAGCAATTTTGCATTGGGGACAACAAATACGTTCCTGACCCCAATGCAGGGGGACTCACAAC
GAATGCTCCTCCTCAAGGACGAGATGTTGTAGAATGGCTAGGTTGGTTTGAAGACCAGAATAGAAAGC
CCACCCCAGACATGTTGCTATATGCAAAGAGAGCAGTCAACTCTCTGCAAGGGTTAAGAGAAAAAACA
GTCGGGAAGTATGCCAAATCTGAGTTTGACAAGTGACCATGTGTTGACAGCCTATACTATAAGATCTG
TTACATATGAAAAAAACTAACAGAAATCATGGACAATCTGGCCAAAGTTAGAGAATATCTTAAGACAT
ATTCCCGTTTAGACCAAGCAGTTCAAGAGATGGACGATTTAGAAGCTCAGAGAGAGGAGAAGACTAAC
TATGAATTATTTCAAGAAGAAGGGATAGATATTCAGAATCATCCATCTTATTATCAGGCAGCAGCAGA
TGAAAGTTCAGATAGTGAAGAAGAAGAGATGTTGGAAGCTTATGATGTCACTGAGGACCAACATAAGA
CTATTTCTACAGATGAGGTAGAGGGTTATATTGCTGAACCATCTGACGATTACGCAGATGATGAAGTC
AACGTAGTTTTCACCTCAGATTGGAAACAACCAGAGCTAGAATCTGATGGAAATGGGAAGACACTGAG
GCTCACAATGCCAGAAGGCTTAAGCAATGAACAGCAATCCCAATGGCTTTCAACCATTAAAGCTGTTG
TGCAGAGTGCAAAGTATTGGAATATTGCAGAGTGTACCCTGGAGAGCACAAAGTCAGGGGTTGTAATG
AAAGAACGACAAATGACCCCTGATGTATATAAGGTGACCCCTGTCCTCAACAATCCTTCATCGATAGA
AGAGACTCCAACAGATGTTTGGTCCCTTTGTCAGGTTTCTGTTTCTTTCACCCCGAGGAAAAGTGGCA
TTCAGCCATTTGTAATCTCTCTGGAGGAATTATTTAACTCACGTGCAGAATTCATCTCAGTTGGCGGC
AACGGAAAAATGTCACATAAAGAGGCAATACTCCTTGGGCTCCGTTATAAAAAGCTATACAATCAAGC
AAGAGTGAAGTATAATCTAGGATGATTATGAAAAAAACTAACAGAGATACAAATGCTCTGAATACTTA
AGTGTTCAAAATGAGCTCCCTCAAGAAAATACTTGGTATTAAGGGAAAGAACAAAAAATCTAAGAAAT
TGGGCCTTCCTCCTCCTCCTTATGAAGAAGATGCAAGGATGGAATTTGCACCTAGTGCCCCAATTGAC
AGATCATTTTTCGGAGTTGAAGATATGGATATCCAGGATAAAAAGCAACTCAGATACGAGAAGTTCTA
TTTTTCAGTCAAGATGACAGTTCGCTCAAATAGACCCTTCAGAACTTACTCTGATGTCGCATCGGCTG
TATCTAACTGGGATCACATGTATATTGGGATGGCAGGAAAAAGACCATTTTATAAAATCTTGGCCTTC
TTGGGATCTACACTCTTGAAAGCAACTCCAGCCGTGCTAGCCGACCACGGACAGCCAGAATATCACGC
CCATTGTGAAGGCCGAGCTTATCTCCCTCATCGATTAGGTCCAACTCCCCCCATGTTGAACGTCCCTG
AACATTTTCGGAGACCTTTTAATATAGGTCTGTTCAGAGGCACTATTGATCTAACAATGACTCTCCAT
GATGATGAGTCTTTGGAGGCCGCACCAATGATTTGGGATCATTTTAATGCATCCCGAGTCACTGATTT
TCCAGAAAAGGCCTTGCTATTTGGACTGATTGTAGAGAAGAAAGCAACGGGGGCTTGGATCTTAGACT
CAATCAGCAATTTCAAATAATCTTCTTTGACAAATTCTCAGCTACATAATTTATTGTGAATCAGTCTT
GCTTACCTTATGAAAAAAACTAACAGAAATCATTCTGTTTCGTCACTATGCTGGTTTTATACCTGTTA
TTGAGCCTTTTGGCTCTGGGAGCTCAATGCAAGTTCACTATAGTATTTCCTCACAATCAAAAAGGGAA
TTGGAAAAATGTACCGGCAAATTATCAGTATTGTCCTTCTAGTTCTGACTTGAATTGGCACAATGGGC
TGATTGGCACTTCTCTCCAAGTCAAAATGCCCAAAAGCCATAAGGCCATCCAAGCGGATGGTTGGATG
TGTCATGCTGCCAAGTGGGTGACTACTTGTGACTTCAGATGGTACGGACCTAAATATGTGACACATTC
TATAAAGTCCATGATACCTACAGTCGACCAGTGTAAAGAAAGTATAGCCCAGACTAAACAAGGAACGT
GGTTAAATCCGGGTTTCCCTCCCCAAAGTTGTGGATATGCTTCCGTTACAGATGCAGAGGCTGTGATA
GTCAAAGCAACCCCCCACCAGGTTTTGGTTGACGAATATACAGGAGAATGGGTTGACTCCCAATTTCC
GACTGGAAAATGCAATAAAGACATTTGCCCAACAGTTCACAACTCAACTACCTGGCACTCAGATTATA
AGGTCACTGGCCTTTGCGATGCAAATTTGATCTCAATGGACATCACTTTCTTCTCCGAAGATGGAAAA
TTAACATCCCTCGGGAAAGAAGGAACAGGGTTCAGAAGCAATTACTTTGCATACGAAAATGGTGACAA
AGCATGCCGCATGCAGTACTGTAAACACTGGGGAGTTCGACTTCCATCCGGAGTGTGGTTCGAAATGG
CAGATAAAGACATCTATAATGATGCGAAATTCCCGGATTGCCCTGAAGGATCATCCATTGCGGCTCCC
TCTCAGACTTCAGTCGATGTTAGTCTCATTCAGGATGTAGAGAGAATCTTGGACTACTCTTTGTGTCA
GGAAACCTGGAGCAAAATTCGTGCTCATTTGCCCATTTCACCAGTTGACCTCAGCTATTTATCCCCAA
AAAATCCTGGAACTGGTCCTGCATTCACTATCATCAATGGGACATTAAAATACTTTGAGACTCGATAC
ATAAGAGTCGATATCGCAGGACCCATCATTCCTCAAATGAGAGGAGTAATCAGCGGAACCACGACCGA
GAGAGAGCTGTGGACGGACTGGTACCCCTACGAAGATGTTGAAATCGGACCAAATGGGGTTTTGAAAA
CTGCTACAGGGTATAAGTTCCCTTTATATATGATTGGCCACGGCATGCTCGACTCAGATCTCCACATC
ACATCAAAGGCTCAGGTTTTTGAACATCCCCATATTCAGGATGCTGCTTCTCAGCTTCCTGATGATGA
GACTTTATTTTTTGGTGATACTGGACTCTCGAAAAACCCCATAGAGCTTGTAGAAGGTTGGTTCAGCG
GATGGAAAAGCACTATTGCTTCTTTTTTCTTCATAATAGGGCTTGTGATCGGATTATATTTGGTTCTT
AGGATTGGAATCGCTTTATGCATCAAATGCCGAGTGCAGGAGAAAAGGCCCAAAATTTACACTGATGT
GGAAATGAACAGATTGGATCGATGAAAGCTTCATTGCTGCAGATGACAACCACACTCACCAAAGATCT
CCAGTTTCAAAGTTATATTGGAGACACAATTTTACAATAATGATGACTACAGGAAACCAAATTATAAT
CACTTCCAAAGTAAACACGACTTAATTTTATGTATGAAAAAAACTAACAGCCATCATGGATGTCAACG
ATTTTGAGATTGATGATCAGGTCACATTTGATGAGGAAGATTATGTTACACAGGAATTCCTCAATCCT
GATGAGAGGATGACCTATCTTAATCATGCAGATTACAATCTGAATTCCCCCTTGATTAGTGATGACAT
CGACAATCTGTTAAGAAGATATAACAGCATGCCGGTACCAAAAATGTGGGAAAACAAGCCATGGGAGG
GAGTGTTGGAAATGCTGACCTCTTGTCAAGCTAACCCTTTGCCATCCACTAAAATTCATAATTGGATG
GGAAGATGGCTCATGTCAGATGACCACGATACCAGCCAAGGTTATAGCTTTCTCCATGAAGTTGATAA
GGAAGCAGAGTTGACCTTCGACATTGTGGAGACTTTCTTGAAAGGATGGGGAGGATGTGTAATAAAAT
TTCAAAAGAAGGAAGGTTTTCATGATGCTTTCAAGGTCGTTGCATCATTGTGCCAAAAATTCTTAGAC
CTACATAAATTAACTCTCATCTTGAATGCTGTCTCTCAAGACGAACTGAATAATCTAAGCAGAACATT
CAAAGGGAAAAATAGAACAGCCAATTCAGGAAACATTATCACCAGATTGAGAGTACCAAGTTTGGGAC
CTGCTTTTGTGACACTGGGCTGGGTGTACTTAAAAAAGTTAGATCTCTTATTTGATCGGAATGTGCTA
CTTATGATCAAAGATGTTATCATTGGACGGATGCAGACTATTCTGTCCATGATTGGAAGGAATGATGC
CTTATTTTCAGAACAAGACATATTCACATTGTTGACAATTTATAGAATTGGAGATCGCATCGTCGAAA
GATTGGGAAATCAAGCGTATGACTTAATCAAAATGGTTGAACCTATATGTAATTTAAAACTGATGAAC
TTGGCTCGAGAGTATCGCCCGCTAGTTCCAAAATTCCCTCACTTTGAACAACACATTCTTCAATCTGT
TCAAGAAGCCAAGAAAATAGATAATGGAATCAGTTATCTGCATGATCAGATCTTAAGTCTCCAGTCTG
TAGATCTGACTCTCGTCGTCTATGGATCTTTCCGACATTGGGGTCATCCATTCATTGATTATTATGCA
GGCCTTGAAAAACTTCATAAGCAGGTCACAATGGAAAAGACAATTGACTCATCTTACGCCAATGCCCT
CGCTAGTGACCTAGCCCGGACTGTCCTACAGAATCAATTCAATGAGCACAAAAAATGGTTTGTAGATG
AAGGCTTGATAAGTGCTGATCATCCCTTCAAAAATCACATTCTGGAAAACACCTGGCCGACTGCAGCT
CAAGTGCAGGATTTTGGGGATAAGTGGCATCATTTGCCCCTTATTAAGTGTTTTGAAATTCCAGATCT
CTTAGATCCGTCAATCATCTATTCAGATAAAAGTCACTCTATGAATAAAAGCGAAGTTCTTCGTCATG
TTAGACTCAATCCCACAACTCCAATACCCAGCAGGAAAGTTTTGCAGACAATGCTAGATACAAAGGCG
ACAGATTGGAAGGAGTTTTTGCAAAACATTGATGAACACGGGTTAGATGAGGATGATCTAGTAATAGG
TCTCAAAGGCAAAGAGCGAGAACTCAAACTAGCCGGTCGATTTTTCTCATTAATGTCATGGAGATTAC
GTGAGTATTTTGTGATCACAGAGTACCTGATCAAGACCCATTTTGTTCCTATGTTTAAAGGGTTGACT
ATGGCAGATGATTTGACAGCTGTCATAAAAAAAATGTTAGACTCTTCTTCAGGCCAGGGACTTGATAA
TTATGAAGCAATTTGCATAGCCAATCATATTGATTACGAAAAATGGAACAACCATCAACGGAAGGAAT
CAAATGGTCCTGTGTTCCGTGTCATGGGCCAGTTTTTAGGTTATCCATCTTTGATTGAGAGAACTCAC
GAATTCTTTGAGAAAAGCTTGATTTATTACAACGGAAGGCCGGATTTGATGAGGGTATCGAACAATAC
ACTTGTTAATGCTACGAACCAAAGAGTCTGTTGGCAGGGACAAGCAGGAGGTTTAGAAGGTCTTAGAC
AAAAAGGATGGAGTATACTGAATCTGCTTGTCATACAAAGAGAGGCAAAAATCCGCAACACTGCAGTA
AAAGTTTTAGCACAAGGAGATAATCAAGTTATTTGCACTCAGTATAAGACAAAAAAATCGAGAAACGA
CCTGGAGCTACGATCTGCGTTAAATCAGATGGTATTGAACAATGAACACATCATGAACGCCATCAAAG
CAGGAACAGGTCGTCTTGGACTTGTGATCAATGATGACGAAACCATGCAGTCAGCTGATTACCTTAAT
TACGGAAAAATTCCAATCTTTCGTGGCGTAATTCGAGGACTCGAAACTAAAAGATGGTCCCGTGTAAC
ATGTGTTACTAACGATCAGATCCCCACATGTGCCAATATCATGAGCTCTGTTTCAACAAATGCATTGA
CCGTAGCACATTTTGCCGAGAATCCCATCAATGCAATGATACAATATAATTATTTCGGGAATTTTGCA
CGACTCTTATTGATGATGCACGATCCAGCACTTCGTAAGTCTCTATATGAGGTACAGTCTTCAATTCC
AGGATTACATAGCGTCACATTCAAGTATGCTATGTTATATCTAGATCCATCCATTGGAGGAGTATCCG
GGATGTCTTTGTCGAGATTCTTAATTCGAGCTTTCCCCGATCCAGTTACTGAGAGCTTATCATTTTGG
AAATTCATTCATGATCACACAAAGGAGGACCACTTAAAAGAGATTAGTGCTGTATTCGGGAATCCAGA
CATTGCACGATTCCGCCTAACACACATAGATAAATTAGTTGAGGATCCTACTTCACTAAATATCGCAA
TGGGCATGAGTCCTGCAAACCTCCTCAAAACAGAAGTCAAAAAATGCTTAATTGAATCCAGACAATCA
ATTAAAAATCAAGTGATCAAAGACGCCACAATTTATCTATACCACGAAGAAGATAAGCTCAGGAGTTT
CCTATGGTCTATCAATCCCTTATTTCCTCGCTTCTTAAGCGAATTCAAATCAGGAACATTTTTGGGTG
TCGCAGATGGTCTGATCAGTTTGTTCCAAAATTCAAGAACAATTCGAAATTCTTTTAGAAGAAAATAT
CATAGGGAGCTTGATGATCTGATAATCAAAAGTGAGGTCTCATCACTCATACACTTAGGAAAGCTACA
TCTTCGAAGAGGCGCTTATCGAATTTGGAGCTGCTCCTCAACCCAGGCTGACACCCTCCGTTATAAAT
CATGGGGTCGTACAGTCATAGGCACAACTGTCCCTCATCCTCTGGAAATGTTAGGACCTCATTCAAAG
AAAGAAGGACCCTGTGTCGCCTGTAATGCATCAGGTTTTAATTATGTCTCAGTGTATTGTCCTTCTGG
AATCAATAATGTCTTCTTATCCCGAGGTCCGCTACCAGCCTATTTGGGTTCTAAAACCTCTGAATCAA
CTTCCATTTTGCAACCTTGGGAACGTGAGAGCAAAGTACCACTGATAAAGAGAGCCACTAGATTAAGA
GATGCTATCTCATGGTTTGTTGACCCCAATTCTAATTTGGCAAAAACTATCCTTGATAATATTCATGC
ATTAACAGGCGAGGAATGGTCAAAGAAACAACATGGATTCAAACGGACAGGATCTGCATTGCATCGAT
TCTCTACCTCAAGAATGAGCCATGGCGGGTTTGCCTCACAGAGCACTGCAGCATTGACAAGATTAATG
GCGACCACAGACACGATGAGGGACTTAGGAGATCAGAACTATGATTTTCTCTTTCAAGCCACTCTTCT
ATATGCTCAAATCACAACAACCGTGGTTCGAAACGGTTATCTCTCCAGTTGCACCGATCACTATCACA
TTACTTGCCGGTCATGCCTCAGGACCATAGAGGAGGTTACATTGGATTCTACTATGGATTACTCACCT
CCAGACGTTTCCCATGTCTTGAAGACTTGGAGAAATGGGGAAGGATCCTGGGGACAAGAAATTAAACA
AATATATCCTGTGGAGGGGGATTGGAAAACTTTATCTCCTGCAGAGCAATCTTATCAAGTGGGAAGAT
GCATCGGATTCCTTTATGGGGACTTAGCATATAGAAAGTCATCTCATGCGGATGACAGCTCTCTTTTT
CCCCTTTCCATACAAAATAGAATCCGTGGAAGAGGTTTCCTCAAAGGACTCCTTGATGGATTAATGAG
GGCGAGTTGTTGTCAGGTGATTCATCGCAGAAGTTTAGCCCATCTAAAACGGCCAGCTAACGCAGTGT
ATGGAGGTCTGATTTATCTCATCGATAAGATTAGTGCTTCTGCACCATTCTTGTCTTTGACCAGATCT
GGTCCTTTACGAAGTGAGTTGGAAACTGTTCCTCACAAGATACCCACTTCTTATCCAACCAGCAATAG
AGATATGGGAATAATTGTTCGAAATTATTTTAAGTACCAATGCAGACAGATTGAAAAGGGCAAGTACA
AGACACACTACACACAATTATGGTTGTTTTCTGATGTACTATCCATCGATTTTCTTGGTCCTTTATCC
ATATCTACAATCCTAATGACTCTACTGTACAAACAATCTCTTTCTGCAAGGGACAAAAATGAACTACG
TGAATTAGCTAATTTGTCATCATTATTGAGATCAGGGGAAGGCTGGGAAGAAGTTCATGTCAAATTCT
TTTCTAGGGATATATTACTTTGTCCAGAAGAAATCAGACATGCCTGCAAATTTGGTATCGCGAAAGAA
ATAAACACAGAGTCCTATTACCCTCCTTGGGAAAAGGAAGTAACAGGACCTATAACAATTCATCCTAT
CTATTACACAACGGTTCCTCATCCGAAGATCTTAGATTCGCCTCCGCGGGTGCAAAACCCTTTGTTAT
CTGGACTAAGATTAGGTCAACTACCAACAGGAGCACATTACAAAATTCGAAGCATTATTCGAGGATTA
AAAATACACTTCAAAGACGTCTTATGTTGCGGAGATGGATCAGGAGGTATGACAGCAGCTTTGTTGAG
AGAAAGTCGACACAGTAGGGCAATTTTCAATAGTTTACTGGAATTGTCCGGTTCCATAATGAGGGGTG
CTTCACCAGAGCCTCCTAGTGCTCTGGAAACCTTGGGTGATGAAAAACGAAGATGTGTAAATGGATCA
ACTTGCTGGGAACATCCATCTGATTTAAGTGACACGAAAACATGGGATTATTTTCTTCAATTGAAAAA
TGGATTAGGGCTTCAGCTCGACTTAATTGTGATGGATATGGAGGTTAGAGATCCAATAATTAGTCAAA
AAATAGAATATAATGTTCGCCATTATATGCATCGACTGCTAGATCAGAATGGAGTCTTAATTTACAAG
ACATATGGCACCTACCTCCAATCCATCACTCAGAACATTCTAACAATTGCCGGACCACTATTTAGGTC
TGTTGATTTGGTTCAGACTGAGTTCAGTAGTTCCCAAACCTCTGAAGTGTATTGTGTCTGCCAGGGAT
TAAAAAACATGATAGATGAACCTCATGTAGATTGGTCATTGCTGAGAGATAGATGGAATCAGCTGTAT
GCATTCCAAACAGAGGAACAGGAATTCATCAGAGCCAAGAAAATGTGTCAGAGAGATACTTTGACCGG
CATCCCAGCTCAATTCATCCCAGACCCGTTTGTCAACTTAGAAACAATCTTGCAAATTTTCGGAGTCC
CTACAGGGGTTGCGCATTCGGCCGCACTTACTGCATCCTCACATCCGAATGAGTTAGTGACAGTTAGT
CTCTTTTTTATGACAATAATATCATATTACAATCTCAACCACCTAAGGAAATCGCCTAGTGTTCCTCC
CCCTCCATCAGATGGAATAGCTCAAAATGTTGGTGTCTCTTTCGTCGGGATAAGTTTATGGTTAAGTC
TCATTGAATTGGACCTTAAACTGTATAAAAGATCATTAAGAGCTATAAGAACTTCTTTCCCGATTAGA
TTGGAGACAGTCAGAGTACTTGACGGTTACAAACTTAGATGGCATGTCCATGGATTGGGAATCCCCAA
AGATTGTCGAATCTCTGACTCATTAGCTGCAATTGGCAACTGGATTCGAGCTTTAGAACTTATCCGGA
ATCAATCAAATCAGAAGCCATTCTGTGAGCGCCTGTTCAACCAGCTTTGTCGTTTAGTAGACTACCAT
CTGAAATGGTCTACACTGAGAGACCAGACGGGTGTCAGGGATTGGTTAACCGGACATGTGTCCATTAA
TGATAAATCTATTTTAATCACAAGGAGTGACGTACATGATGAGAACTCTTGGAGATCTTAAAGATGTC
TATATCAATTTGAAGAGCAAAAGCTACAGTATGAAAAAAACTGATAAGACTTAGAACCCTCTTAGGAT
TTTTTTTGTTTTAAATGGTTTGTTGGTTT
Recombinant MORV genome (codon optimized with chimeric trailer sequence and
modified intergenic regions)
SEQ ID NO: 6
AGACAAACAAACCATTATTACAATTAAAAGGCTCAGGAGAACCTTCAACAGCAATCGAAATGTCGGTT
ACAGTCAAAAGAATCATTGACAACTCTGTCATCCTCCCCAAATTGCCAGCCAATGAGGATCCAGTTGA
ATATCCGGGTGACTATTTCAAAAAGACAAATGAGGTCCCTGTGTACATCAACACCAGCAAGACTCTAA
ATGATCTGAGAGGGTATGTTTATCAAGGACTAAAAACAGGCAATGTGTCTATAATCCATGTCAACAGC
TATCTGTATGCAGCATTAAGAGACATTAAAGGAAAACTCGACAAAGATTGGATCAGCTTCGGAGTTCA
GATCGGGAAAACCGGAGATGAAGTTGGAATATTCAATTTAGTGTCAGTCAAAACTCTGGAAGGAATTA
TTCCAGATGGAGTATCAGATGCATCAAGAACTAGCGCAGATGATGCCTGGCTTCCCCTTTATTTACTG
GGGCTTTACAGAGTGGGGAGGACTCAAATGCCAGAATACCGGAAGAAATTGATGGATGGTCTAATTAA
TCAGTGCAAAATGATCAATGAGAAGTTCGAACCTCTAGTTCCTGAGGGGCGTGACATCTTTGATGTGT
GGGGAAACGACAGCAATTATACCAAAATCGTAGCCGCCGTCGACATGTTCTTTCATATGTTCAAAAAG
CATGAGAAGGCCTCTTTCAGATATGGAACTATAGTGTCCAGGTTCAAGGACTGCGCCGCATTGGCGAC
ATTTGGACATCTATGTAAAATCACTGGCATGTCTACAGAAGATGTGACCACTTGGATCCTCAATAGAG
AAGTGGCAGATGAAATGGTTCAAATGATGCTTCCAGGCCAAGAAATTGATAAGGCGGATTCTTACATG
CCTTACCTCATAGACTTCGGACTGTCTTCAAAGTCTCCATACTCGTCCGTAAAAAACCCGGCTTTCCA
CTTTTGGGGTCAATTGACTGCATTGCTGTTGAGATCAACTCGAGCAAGGAATGCCAGACAACCTGACG
ACATCGAGTATACATCATTGACAACAGCAGGTCTCCTATATGCTTATGCAGTAGGTTCATCAGCAGAC
CTTGCGCAGCAATTTTGCATTGGGGACAACAAATACGTTCCTGACCCCAATGCAGGGGGACTCACAAC
GAATGCTCCTCCTCAAGGACGAGATGTTGTAGAATGGCTAGGTTGGTTTGAAGACCAGAATAGAAAGC
CCACCCCAGACATGTTGCTATATGCAAAGAGAGCAGTCAACTCTCTGCAAGGGTTAAGAGAAAAAACA
GTCGGGAAGTATGCCAAATCTGAGTTTGACAAGTGACCATGTGTTGACAGCCTATACTATAAGATCTG
TTACATATGAAAAAAACTAACAGAAATCATGGACAATCTGGCCAAAGTTAGAGAATATCTTAAGACAT
ATTCCCGTTTAGACCAAGCAGTTCAAGAGATGGACGATTTAGAAGCTCAGAGAGAGGAGAAGACTAAC
TATGAATTATTTCAAGAAGAAGGGATAGATATTCAGAATCATCCATCTTATTATCAGGCAGCAGCAGA
TGAAAGTTCAGATAGTGAAGAAGAAGAGATGTTGGAAGCTTATGATGTCACTGAGGACCAACATAAGA
CTATTTCTACAGATGAGGTAGAGGGTTATATTGCTGAACCATCTGACGATTACGCAGATGATGAAGTC
AACGTAGTTTTCACCTCAGATTGGAAACAACCAGAGCTAGAATCTGATGGAAATGGGAAGACACTGAG
GCTCACAATGCCAGAAGGCTTAAGCAATGAACAGCAATCCCAATGGCTTTCAACCATTAAAGCTGTTG
TGCAGAGTGCAAAGTATTGGAATATTGCAGAGTGTACCCTGGAGAGCACAAAGTCAGGGGTTGTAATG
AAAGAACGACAAATGACCCCTGATGTATATAAGGTGACCCCTGTCCTCAACAATCCTTCATCGATAGA
AGAGACTCCAACAGATGTTTGGTCCCTTTGTCAGGTTTCTGTTTCTTTCACCCCGAGGAAAAGTGGCA
TTCAGCCATTTGTAATCTCTCTGGAGGAATTATTTAACTCACGTGCAGAATTCATCTCAGTTGGCGGC
AACGGAAAAATGTCACATAAAGAGGCAATACTCCTTGGGCTCCGTTATAAAAAGCTATACAATCAAGC
AAGAGTGAAGTATAATCTAGGATGATTATGAAAAAAACTAACAGAGATCCAAATGCTCTGAATACTTA
AGTGTTCAAAATGAGCTCCCTCAAGAAAATACTTGGTATTAAGGGAAAGAACAAAAAATCTAAGAAAT
TGGGCCTTCCTCCTCCTCCTTATGAAGAAGATGCAAGGATGGAATTTGCACCTAGTGCCCCAATTGAC
AGATCATTTTTCGGAGTTGAAGATATGGATATCCAGGATAAAAAGCAACTCAGATACGAGAAGTTCTA
TTTTTCAGTCAAGATGACAGTTCGCTCAAATAGACCCTTCAGAACTTACTCTGATGTCGCATCGGCTG
TATCTAACTGGGATCACATGTATATTGGGATGGCAGGAAAAAGACCATTTTATAAAATCTTGGCCTTC
TTGGGATCTACACTCTTGAAAGCAACTCCAGCCGTGCTAGCCGACCACGGACAGCCAGAATATCACGC
CCATTGTGAAGGCCGAGCTTATCTCCCTCATCGATTAGGTCCAACTCCCCCCATGTTGAACGTCCCTG
AACATTTTCGGAGACCTTTTAATATAGGTCTGTTCAGAGGCACTATTGATCTAACAATGACTCTCCAT
GATGATGAGTCTTTGGAGGCCGCACCAATGATTTGGGATCATTTTAATGCATCCCGAGTCACTGATTT
TCCAGAAAAGGCCTTGCTATTTGGACTGATTGTAGAGAAGAAAGCAACGGGGGCTTGGATCTTAGACT
CAATCAGCAATTTCAAATAATCTTCTTTGACAAATTCTCAGCTACATAATTTATTGTGAATCAGTCTT
GCTTACCTTATGAAAAAAACTAACAGAAATCATTCTGTTTCGTCACTATGCTGGTTTTATACCTGTTA
TTGAGCCTTTTGGCTCTGGGAGCTCAATGCAAGTTCACTATAGTATTTCCTCACAATCAAAAAGGGAA
TTGGAAAAATGTACCGGCAAATTATCAGTATTGTCCTTCTAGTTCTGACTTGAATTGGCACAATGGGC
TGATTGGCACTTCTCTCCAAGTCAAAATGCCCAAAAGCCATAAGGCCATCCAAGCGGATGGTTGGATG
TGTCATGCTGCCAAGTGGGTGACTACTTGTGACTTCAGATGGTACGGACCTAAATATGTGACACATTC
TATAAAGTCCATGATACCTACAGTCGACCAGTGTAAAGAAAGTATAGCCCAGACTAAACAAGGAACGT
GGTTAAATCCGGGTTTCCCTCCCCAAAGTTGTGGATATGCTTCCGTTACAGATGCAGAGGCTGTGATA
GTCAAAGCAACCCCCCACCAGGTTTTGGTTGACGAATATACAGGAGAATGGGTTGACTCCCAATTTCC
GACTGGAAAATGCAATAAAGACATTTGCCCAACAGTTCACAACTCAACTACCTGGCACTCAGATTATA
AGGTCACTGGCCTTTGCGATGCAAATTTGATCTCAATGGACATCACTTTCTTCTCCGAAGATGGAAAA
TTAACATCCCTCGGGAAAGAAGGAACAGGGTTCAGAAGCAATTACTTTGCATACGAAAATGGTGACAA
AGCATGCCGCATGCAGTACTGTAAACACTGGGGAGTTCGACTTCCATCCGGAGTGTGGTTCGAAATGG
CAGATAAAGACATCTATAATGATGCGAAATTCCCGGATTGCCCTGAAGGATCATCCATTGCGGCTCCC
TCTCAGACTTCAGTCGATGTTAGTCTCATTCAGGATGTAGAGAGAATCTTGGACTACTCTTTGTGTCA
GGAAACCTGGAGCAAAATTCGTGCTCATTTGCCCATTTCACCAGTTGACCTCAGCTATTTATCCCCAA
AAAATCCTGGAACTGGTCCTGCATTCACTATCATCAATGGGACATTAAAATACTTTGAGACTCGATAC
ATAAGAGTCGATATCGCAGGACCCATCATTCCTCAAATGAGAGGAGTAATCAGCGGAACCACGACCGA
GAGAGAGCTGTGGACGGACTGGTACCCCTACGAAGATGTTGAAATCGGACCAAATGGGGTTTTGAAAA
CTGCTACAGGGTATAAGTTCCCTTTATATATGATTGGCCACGGCATGCTCGACTCAGATCTCCACATC
ACATCAAAGGCTCAGGTTTTTGAACATCCCCATATTCAGGATGCTGCTTCTCAGCTTCCTGATGATGA
GACTTTATTTTTTGGTGATACTGGACTCTCGAAAAACCCCATAGAGCTTGTAGAAGGTTGGTTCAGCG
GATGGAAAAGCACTATTGCTTCTTTTTTCTTCATAATAGGGCTTGTGATCGGATTATATTTGGTTCTT
AGGATTGGAATCGCTTTATGCATCAAATGCCGAGTGCAGGAGAAAAGGCCCAAAATTTACACTGATGT
GGAAATGAACAGATTGGATCGATGAAAGCTTCATTGCTGCAGATGACAACCACACTCACCAAAGATCT
CCAGTTTCAAAGTTATATTGGAGACACAATTTTACAATAATGATGACTACAGGAAACCAAATTATAAT
CACTTCCAAAGTAAACACGACTTAATTTTATGTATGAAAAAAACTAACAGCCATCATGGATGTCAACG
ATTTTGAGATTGATGATCAGGTCACATTTGATGAGGAAGATTATGTTACACAGGAATTCCTCAATCCT
GATGAGAGGATGACCTATCTTAATCATGCAGATTACAATCTGAATTCCCCCTTGATTAGTGATGACAT
CGACAATCTGTTAAGAAGATATAACAGCATGCCGGTACCAAAAATGTGGGAAAACAAGCCATGGGAGG
GAGTGTTGGAAATGCTGACCTCTTGTCAAGCTAACCCTTTGCCATCCACTAAAATTCATAATTGGATG
GGAAGATGGCTCATGTCAGATGACCACGATACCAGCCAAGGTTATAGCTTTCTCCATGAAGTTGATAA
GGAAGCAGAGTTGACCTTCGACATTGTGGAGACTTTCTTGAAAGGATGGGGAGGATGTGTAATAAAAT
TTCAAAAGAAGGAAGGTTTTCATGATGCTTTCAAGGTCGTTGCATCATTGTGCCAAAAATTCTTAGAC
CTACATAAATTAACTCTCATCTTGAATGCTGTCTCTCAAGACGAACTGAATAATCTAAGCAGAACATT
CAAAGGGAAAAATAGAACAGCCAATTCAGGAAACATTATCACCAGATTGAGAGTACCAAGTTTGGGAC
CTGCTTTTGTGACACTGGGCTGGGTGTACTTAAAAAAGTTAGATCTCTTATTTGATCGGAATGTGCTA
CTTATGATCAAAGATGTTATCATTGGACGGATGCAGACTATTCTGTCCATGATTGGAAGGAATGATGC
CTTATTTTCAGAACAAGACATATTCACATTGTTGACAATTTATAGAATTGGAGATCGCATCGTCGAAA
GATTGGGAAATCAAGCGTATGACTTAATCAAAATGGTTGAACCTATATGTAATTTAAAACTGATGAAC
TTGGCTCGAGAGTATCGCCCGCTAGTTCCAAAATTCCCTCACTTTGAACAACACATTCTTCAATCTGT
TCAAGAAGCCAAGAAAATAGATAATGGAATCAGTTATCTGCATGATCAGATCTTAAGTCTCCAGTCTG
TAGATCTGACTCTCGTCGTCTATGGATCTTTCCGACATTGGGGTCATCCATTCATTGATTATTATGCA
GGCCTTGAAAAACTTCATAAGCAGGTCACAATGGAAAAGACAATTGACTCATCTTACGCCAATGCCCT
CGCTAGTGACCTAGCCCGGACTGTCCTACAGAATCAATTCAATGAGCACAAAAAATGGTTTGTAGATG
AAGGCTTGATAAGTGCTGATCATCCCTTCAAAAATCACATTCTGGAAAACACCTGGCCGACTGCAGCT
CAAGTGCAGGATTTTGGGGATAAGTGGCATCATTTGCCCCTTATTAAGTGTTTTGAAATTCCAGATCT
CTTAGATCCGTCAATCATCTATTCAGATAAAAGTCACTCTATGAATAAAAGCGAAGTTCTTCGTCATG
TTAGACTCAATCCCACAACTCCAATACCCAGCAGGAAAGTTTTGCAGACAATGCTAGATACAAAGGCG
ACAGATTGGAAGGAGTTTTTGCAAAACATTGATGAACACGGGTTAGATGAGGATGATCTAGTAATAGG
TCTCAAAGGCAAAGAGCGAGAACTCAAACTAGCCGGTCGATTTTTCTCATTAATGTCATGGAGATTAC
GTGAGTATTTTGTGATCACAGAGTACCTGATCAAGACCCATTTTGTTCCTATGTTTAAAGGGTTGACT
ATGGCAGATGATTTGACAGCTGTCATAAAAAAAATGTTAGACTCTTCTTCAGGCCAGGGACTTGATAA
TTATGAAGCAATTTGCATAGCCAATCATATTGATTACGAAAAATGGAACAACCATCAACGGAAGGAAT
CAAATGGTCCTGTGTTCCGTGTCATGGGCCAGTTTTTAGGTTATCCATCTTTGATTGAGAGAACTCAC
GAATTCTTTGAGAAAAGCTTGATTTATTACAACGGAAGGCCGGATTTGATGAGGGTATCGAACAATAC
ACTTGTTAATGCTACGAACCAAAGAGTCTGTTGGCAGGGACAAGCAGGAGGTTTAGAAGGTCTTAGAC
AAAAAGGATGGAGTATACTGAATCTGCTTGTCATACAAAGAGAGGCAAAAATCCGCAACACTGCAGTA
AAAGTTTTAGCACAAGGAGATAATCAAGTTATTTGCACTCAGTATAAGACAAAAAAATCGAGAAACGA
CCTGGAGCTACGATCTGCGTTAAATCAGATGGTATTGAACAATGAACACATCATGAACGCCATCAAAG
CAGGAACAGGTCGTCTTGGACTTGTGATCAATGATGACGAAACCATGCAGTCAGCTGATTACCTTAAT
TACGGAAAAATTCCAATCTTTCGTGGCGTAATTCGAGGACTCGAAACTAAAAGATGGTCCCGTGTAAC
ATGTGTTACTAACGATCAGATCCCCACATGTGCCAATATCATGAGCTCTGTTTCAACAAATGCATTGA
CCGTAGCACATTTTGCCGAGAATCCCATCAATGCAATGATACAATATAATTATTTCGGGAATTTTGCA
CGACTCTTATTGATGATGCACGATCCAGCACTTCGTAAGTCTCTATATGAGGTACAGTCTTCAATTCC
AGGATTACATAGCGTCACATTCAAGTATGCTATGTTATATCTAGATCCATCCATTGGAGGAGTATCCG
GGATGTCTTTGTCGAGATTCTTAATTCGAGCTTTCCCCGATCCAGTTACTGAGAGCTTATCATTTTGG
AAATTCATTCATGATCACACAAAGGAGGACCACTTAAAAGAGATTAGTGCTGTATTCGGGAATCCAGA
CATTGCACGATTCCGCCTAACACACATAGATAAATTAGTTGAGGATCCTACTTCACTAAATATCGCAA
TGGGCATGAGTCCTGCAAACCTCCTCAAAACAGAAGTCAAAAAATGCTTAATTGAATCCAGACAATCA
ATTAAAAATCAAGTGATCAAAGACGCCACAATTTATCTATACCACGAAGAAGATAAGCTCAGGAGTTT
CCTATGGTCTATCAATCCCTTATTTCCTCGCTTCTTAAGCGAATTCAAATCAGGAACATTTTTGGGTG
TCGCAGATGGTCTGATCAGTTTGTTCCAAAATTCAAGAACAATTCGAAATTCTTTTAGAAGAAAATAT
CATAGGGAGCTTGATGATCTGATAATCAAAAGTGAGGTCTCATCACTCATACACTTAGGAAAGCTACA
TCTTCGAAGAGGCGCTTATCGAATTTGGAGCTGCTCCTCAACCCAGGCTGACACCCTCCGTTATAAAT
CATGGGGTCGTACAGTCATAGGCACAACTGTCCCTCATCCTCTGGAAATGTTAGGACCTCATTCAAAG
AAAGAAGGACCCTGTGTCGCCTGTAATGCATCAGGTTTTAATTATGTCTCAGTGTATTGTCCTTCTGG
AATCAATAATGTCTTCTTATCCCGAGGTCCGCTACCAGCCTATTTGGGTTCTAAAACCTCTGAATCAA
CTTCCATTTTGCAACCTTGGGAACGTGAGAGCAAAGTACCACTGATAAAGAGAGCCACTAGATTAAGA
GATGCTATCTCATGGTTTGTTGACCCCAATTCTAATTTGGCAAAAACTATCCTTGATAATATTCATGC
ATTAACAGGCGAGGAATGGTCAAAGAAACAACATGGATTCAAACGGACAGGATCTGCATTGCATCGAT
TCTCTACCTCAAGAATGAGCCATGGCGGGTTTGCCTCACAGAGCACTGCAGCATTGACAAGATTAATG
GCGACCACAGACACGATGAGGGACTTAGGAGATCAGAACTATGATTTTCTCTTTCAAGCCACTCTTCT
ATATGCTCAAATCACAACAACCGTGGTTCGAAACGGTTATCTCTCCAGTTGCACCGATCACTATCACA
TTACTTGCCGGTCATGCCTCAGGACCATAGAGGAGGTTACATTGGATTCTACTATGGATTACTCACCT
CCAGACGTTTCCCATGTCTTGAAGACTTGGAGAAATGGGGAAGGATCCTGGGGACAAGAAATTAAACA
AATATATCCTGTGGAGGGGGATTGGAAAACTTTATCTCCTGCAGAGCAATCTTATCAAGTGGGAAGAT
GCATCGGATTCCTTTATGGGGACTTAGCATATAGAAAGTCATCTCATGCGGATGACAGCTCTCTTTTT
CCCCTTTCCATACAAAATAGAATCCGTGGAAGAGGTTTCCTCAAAGGACTCCTTGATGGATTAATGAG
GGCGAGTTGTTGTCAGGTGATTCATCGCAGAAGTTTAGCCCATCTAAAACGGCCAGCTAACGCAGTGT
ATGGAGGTCTGATTTATCTCATCGATAAGATTAGTGCTTCTGCACCATTCTTGTCTTTGACCAGATCT
GGTCCTTTACGAAGTGAGTTGGAAACTGTTCCTCACAAGATACCCACTTCTTATCCAACCAGCAATAG
AGATATGGGAATAATTGTTCGAAATTATTTTAAGTACCAATGCAGACAGATTGAAAAGGGCAAGTACA
AGACACACTACACACAATTATGGTTGTTTTCTGATGTACTATCCATCGATTTTCTTGGTCCTTTATCC
ATATCTACAATCCTAATGACTCTACTGTACAAACAATCTCTTTCTGCAAGGGACAAAAATGAACTACG
TGAATTAGCTAATTTGTCATCATTATTGAGATCAGGGGAAGGCTGGGAAGAAGTTCATGTCAAATTCT
TTTCTAGGGATATATTACTTTGTCCAGAAGAAATCAGACATGCCTGCAAATTTGGTATCGCGAAAGAA
ATAAACACAGAGTCCTATTACCCTCCTTGGGAAAAGGAAGTAACAGGACCTATAACAATTCATCCTAT
CTATTACACAACGGTTCCTCATCCGAAGATCTTAGATTCGCCTCCGCGGGTGCAAAACCCTTTGTTAT
CTGGACTAAGATTAGGTCAACTACCAACAGGAGCACATTACAAAATTCGAAGCATTATTCGAGGATTA
AAAATACACTTCAAAGACGTCTTATGTTGCGGAGATGGATCAGGAGGTATGACAGCAGCTTTGTTGAG
AGAAAGTCGACACAGTAGGGCAATTTTCAATAGTTTACTGGAATTGTCCGGTTCCATAATGAGGGGTG
CTTCACCAGAGCCTCCTAGTGCTCTGGAAACCTTGGGTGATGAAAAACGAAGATGTGTAAATGGATCA
ACTTGCTGGGAACATCCATCTGATTTAAGTGACACGAAAACATGGGATTATTTTCTTCAATTGAAAAA
TGGATTAGGGCTTCAGCTCGACTTAATTGTGATGGATATGGAGGTTAGAGATCCAATAATTAGTCAAA
AAATAGAATATAATGTTCGCCATTATATGCATCGACTGCTAGATCAGAATGGAGTCTTAATTTACAAG
ACATATGGCACCTACCTCCAATCCATCACTCAGAACATTCTAACAATTGCCGGACCACTATTTAGGTC
TGTTGATTTGGTTCAGACTGAGTTCAGTAGTTCCCAAACCTCTGAAGTGTATTGTGTCTGCCAGGGAT
TAAAAAACATGATAGATGAACCTCATGTAGATTGGTCATTGCTGAGAGATAGATGGAATCAGCTGTAT
GCATTCCAAACAGAGGAACAGGAATTCATCAGAGCCAAGAAAATGTGTCAGAGAGATACTTTGACCGG
CATCCCAGCTCAATTCATCCCAGACCCGTTTGTCAACTTAGAAACAATCTTGCAAATTTTCGGAGTCC
CTACAGGGGTTGCGCATTCGGCCGCACTTACTGCATCCTCACATCCGAATGAGTTAGTGACAGTTAGT
CTCTTTTTTATGACAATAATATCATATTACAATCTCAACCACCTAAGGAAATCGCCTAGTGTTCCTCC
CCCTCCATCAGATGGAATAGCTCAAAATGTTGGTGTCTCTTTCGTCGGGATAAGTTTATGGTTAAGTC
TCATTGAATTGGACCTTAAACTGTATAAAAGATCATTAAGAGCTATAAGAACTTCTTTCCCGATTAGA
TTGGAGACAGTCAGAGTACTTGACGGTTACAAACTTAGATGGCATGTCCATGGATTGGGAATCCCCAA
AGATTGTCGAATCTCTGACTCATTAGCTGCAATTGGCAACTGGATTCGAGCTTTAGAACTTATCCGGA
ATCAATCAAATCAGAAGCCATTCTGTGAGCGCCTGTTCAACCAGCTTTGTCGTTTAGTAGACTACCAT
CTGAAATGGTCTACACTGAGAGACCAGACGGGTGTCAGGGATTGGTTAACCGGACATGTGTCCATTAA
TGATAAATCTATTTTAATCACAAGGAGTGACGTACATGATGAGAACTCTTGGAGATCTTAAGATGTCT
ATATCAATTTGAAGAGCAAAAGCTACAGTATGAAAAAAACTGATAAGACTTAGAACCCTCTTAGGATT
TTTTTTGTTTTAAATGGTTTGTTGGTTTGGCATGGCATCTCCACC
first section of plain text: chimeric leader sequence
first section of bold text: MORV-N gene
first section of underlined text: first intergenic region
second section of bold text: MORV-P gene
second section of underlined text: second intergenic region
third section of bold text: MORV-M gene
third section of underlined text: third intergenic region
fourth section of bold text: MORV-G gene
fourth section of underlined text: fourth intergenic region
fifth section of bold text: MORV-L gene
fifth section of underlined text: fifth intergenic region
second section of plain text: chimeric trailer sequence

### Example 3: Treating Cancer

A human identified as having a liver cancer is administered a MORV that includes a genome having a nucleic acid sequence set forth in SEQ ID NO:5. The MORV that includes a genome having the nucleic acid sequence set forth in SEQ ID NO:5 can reduce the size of the liver cancer in the human.

### Example 4: Treating Cancer

A human identified as having a liver cancer is administered a MORV that includes a genome having a nucleic acid sequence set forth in SEQ ID NO:6. The MORV having that includes a genome having the nucleic acid sequence set forth in SEQ ID NO:6 can reduce the size of the liver cancer in the human.

### Example 5: Exemplary Embodiments

Embodiment 1. A recombinant Morreton virus (MORV) comprising:
(a) a leader sequence of a first Vesiculovirus species different from said MORV;
(b) a synthetic intergenic region; and
(c) a trailer sequence of a second Vesiculovirus species different from said MORV.
   Embodiment 2. The recombinant MORV of embodiment 1, wherein said first Vesiculovirus species is a vesicular stomatitis virus (VSV).
   Embodiment 3. The recombinant MORV of embodiment 2, wherein said leader sequence comprises the sequence set forth in SEQ ID NO:3.
   Embodiment 4. The recombinant MORV of embodiment 3, wherein said leader sequence consists of said sequence set forth in SEQ ID NO:3.
   Embodiment 5. The recombinant MORV of any one of embodiments 1-4, wherein said leader sequence is located before a MORV-N gene within the MORV genome.
   Embodiment 6. The recombinant MORV of embodiment 1, wherein said synthetic intergenic region comprises a sequence set forth in SEQ ID NO:1 or SEQ ID NO:2.
   Embodiment 7. The recombinant MORV of embodiment 6, wherein said synthetic intergenic region consists of said sequence set forth in SEQ ID NO:2.
   Embodiment 8. The recombinant MORV of any one of embodiments 6-7, wherein said synthetic intergenic region is located after a stop of a first gene within said MORV genome and before a start codon of a second gene within said MORV genome.
   Embodiment 9. The recombinant MORV of any one of embodiments 6-7, where said synthetic intergenic region is located between each gene within the MORV genome.
   Embodiment 10. The recombinant MORV of embodiment 1, wherein said second Vesiculovirus species is a VSV.
   Embodiment 11. The recombinant MORV of embodiment 10, wherein said trailer sequence comprises a sequence set forth in SEQ ID NO:4.
   Embodiment 12. The recombinant MORV of embodiment 11, wherein said trailer sequence consists of said sequence set forth in SEQ ID NO:4.
   Embodiment 13. The recombinant MORV of any one of embodiments 11-12, wherein said trailer sequence is located after a MORV-L gene within the MORV genome.
   Embodiment 14. The recombinant MORV of any one of embodiments 1-13, wherein a genome of said recombinant MORV comprises:
(a) said leader sequence before a MORV-N gene within said genome;
(b) said synthetic intergenic region (i) after a stop codon of said MORV-N gene and before a start codon of a MORV-P gene, (ii) after a stop codon of said MORV-P gene and before a start codon of a MORV-M gene, (iii) after a stop codon of said MORV-M gene and before a start codon of a MORV-G gene, (iv) after a stop codon of said MORV-G gene and before a start codon of a MORV-L gene, and (v) after a stop codon of a MORV-L gene; and
(c) said trailer sequence after said MORV-L gene.
   Embodiment 15. The recombinant MORV of embodiment 1, wherein a genome of said recombinant MORV comprises the nucleic acid sequence set forth in SEQ ID NO:5.
   Embodiment 16. The recombinant MORV of embodiment 1, wherein a genome of said recombinant MORV comprises the nucleic acid sequence set forth in SEQ ID NO:6.
   Embodiment 17. A method for treating a mammal having cancer, wherein said method comprises administering, to said mammal, a composition comprising MORV, wherein the number of cancer cells within said mammal is reduced.
   Embodiment 18. The method of embodiment 17, wherein said mammal is a human.
   Embodiment 19. The method of any one of embodiments 17-18, wherein said cancer is selected from the group consisting of a liver cancer, a pancreatic cancer, a breast cancer, a prostate cancer, a bladder cancer, a colorectal cancer, a lungs cancer, a thyroid cancer, a melanoma, a myeloma, and a sarcoma.
   Embodiment 20. The method of embodiment 19, wherein said liver cancer is a cholangiocarcinoma or a hepatocellular carcinoma.
   Embodiment 21. The method of any one of embodiments 17-20, wherein said MORV is a wild-type MORV.
   Embodiment 22. The method of any one of embodiments 17-20, wherein said MORV is a recombinant MORV.
   Embodiment 23. The method of embodiment 22, wherein said recombinant MORV comprises:
(a) a leader sequence of a first Vesiculovirus species different from said MORV;
(b) a synthetic intergenic region; and
(c) a trailer sequence of a second Vesiculovirus species different from said MORV.
   Embodiment 24. The recombinant MORV of embodiment 23, wherein said first Vesiculovirus species is a VSV.
   Embodiment 25. The recombinant MORV of any one of embodiments 23-24, wherein said leader sequence comprises the sequence set forth in SEQ ID NO:3.
   Embodiment 26. The recombinant MORV of embodiment 25, wherein said leader sequence consists of said sequence set forth in SEQ ID NO:3.
   Embodiment 27. The recombinant MORV of any one of embodiments 23-26, wherein said leader sequence is located before a MORV-N gene within the MORV genome.
   Embodiment 28. The recombinant MORV of embodiment 23, wherein said synthetic intergenic region comprises a sequence set forth in SEQ ID NO:1 or SEQ ID NO:2.
   Embodiment 29. The recombinant MORV of embodiment 28, wherein said synthetic intergenic region consists of said sequence set forth in SEQ ID NO:2.
   Embodiment 30. The recombinant MORV of any one of embodiments 28-29, wherein said synthetic intergenic region is located after a stop of a first gene within said MORV genome and before a start codon of a second gene within said MORV genome.
   Embodiment 31. The recombinant MORV of any one of embodiments 28-29, where said synthetic intergenic region is located between each gene within the MORV genome.
   Embodiment 32. The recombinant MORV of embodiment 23, wherein said second Vesiculovirus species is a VSV.
   Embodiment 33. The recombinant MORV of embodiment 32, wherein said trailer sequence comprises a sequence set forth in SEQ ID NO:4.
   Embodiment 34. The recombinant MORV of embodiment 33, wherein said trailer sequence consists of said sequence set forth in SEQ ID NO:4.
   Embodiment 35. The recombinant MORV of any one of embodiments 33-34, wherein said trailer sequence is located after a MORV-L gene within the MORV genome.
   Embodiment 36. The recombinant MORV of any one of embodiments 23-35, wherein a genome of said recombinant MORV comprises:
(a) said leader sequence before a MORV-N gene within said genome;
(b) said synthetic intergenic region (i) after a stop codon of said MORV-N gene and before a start codon of a MORV-P gene, (ii) after a stop codon of said MORV-P gene and before a start codon of a MORV-M gene, (iii) after a stop codon of said MORV-M gene and before a start codon of a MORV-G gene, (iv) after a stop codon of said MORV-G gene and before a start codon of a MORV-L gene, and (v) after a stop codon of a MORV-L gene; and
(c) said trailer sequence after said MORV-L gene.
   Embodiment 37. The recombinant MORV of embodiment 23, wherein a genome of said recombinant MORV comprises the nucleic acid sequence set forth in SEQ ID NO:5.
   Embodiment 38. The recombinant MORV of embodiment 23, wherein a genome of said recombinant MORV comprises the nucleic acid sequence set forth in SEQ ID NO:6.
   Embodiment 39. A use of a composition comprising a MORV for treating a mammal having cancer.
   Embodiment 40. The use of any embodiment 39, wherein said mammal is a human.
   Embodiment 41. The use of any one of embodiments 39-40, wherein said cancer is selected from the group consisting of a liver cancer, a pancreatic cancer, a breast cancer, a prostate cancer, a bladder cancer, a colorectal cancer, a lungs cancer, a thyroid cancer, a melanoma, a myeloma, and a sarcoma.
   Embodiment 42. The use of any one of embodiments 39-41, wherein said MORV is a wild-type MORV.
   Embodiment 43. The use of any one of embodiments 39-41, wherein said MORV is a recombinant MORV Embodiment 44. The use of embodiment 43, wherein said recombinant MORV comprises:
(a) a leader sequence of a first Vesiculovirus species different from said MORV;
(b) a synthetic intergenic region; and
(c) a trailer sequence of a second Vesiculovirus species different from said MORV Embodiment 45. The use of embodiment 44, wherein said first Vesiculovirus species is a VSV.
   Embodiment 46. The use of embodiment 44, wherein said leader sequence comprises the sequence set forth in SEQ ID NO:3.
   Embodiment 47. The use of embodiment 45, wherein said leader sequence consists of said sequence set forth in SEQ ID NO:3.
   Embodiment 48. The use of any one of embodiments 44-47, wherein said leader sequence is located before a MORV-N gene within the MORV genome.
   Embodiment 49. The use of embodiment 44, wherein said synthetic intergenic region comprises a sequence set forth in SEQ ID NO:1 or SEQ ID NO:2.
   Embodiment 50. The use of embodiment 49, wherein said synthetic intergenic region consists of said sequence set forth in SEQ ID NO:2.
   Embodiment 51. The use of any one of embodiments 49-50, wherein said synthetic intergenic region is located after a stop of a first gene within said MORV genome and before a start codon of a second gene within said MORV genome.
   Embodiment 52. The use of any one of embodiments 49-50, where said synthetic intergenic region is located between each gene within the MORV genome.
   Embodiment 53. The use of embodiment 44, wherein said second Vesiculovirus species is a VSV.
   Embodiment 54. The use of embodiment 44, wherein said trailer sequence comprises a sequence set forth in SEQ ID NO:4.
   Embodiment 55. The use of embodiment 44, wherein said trailer sequence consists of said sequence set forth in SEQ ID NO:4.
   Embodiment 56. The use of any one of embodiments 44-55, wherein said trailer sequence is located after a MORV-L gene within the MORV genome.
   Embodiment 57. The use of any one of embodiments 44-56, wherein a genome of said recombinant MORV comprises:
(a) said leader sequence before a MORV-N gene within said genome;
(b) said synthetic intergenic region (i) after a stop codon of said MORV-N gene and before a start codon of a MORV-P gene, (ii) after a stop codon of said MORV-P gene and before a start codon of a MORV-M gene, (iii) after a stop codon of said MORV-M gene and before a start codon of a MORV-G gene, (iv) after a stop codon of said MORV-G gene and before a start codon of a MORV-L gene, and (v) after a stop codon of a MORV-L gene; and
(c) said trailer sequence after said MORV-L gene.
   Embodiment 58. The use of embodiment 43, wherein a genome of said recombinant MORV comprises the nucleic acid sequence set forth in SEQ ID NO:5.
   Embodiment 59. The use of embodiment 43, wherein a genome of said recombinant MORV comprises the nucleic acid sequence set forth in SEQ ID NO:6.
   Embodiment 60. A use of a MORV in the manufacture of a medicament for treating a mammal having cancer.
   Embodiment 61. The use of any embodiment 60, wherein said mammal is a human.
   Embodiment 62. The use of any one of embodiments 60-61, wherein said cancer is selected from the group consisting of a liver cancer, a pancreatic cancer, a breast cancer, a prostate cancer, a bladder cancer, a colorectal cancer, a lungs cancer, a thyroid cancer, a melanoma, a myeloma, and a sarcoma.
   Embodiment 63. The use of any one of embodiments 60-62, wherein said MORV is a wild-type MORV.
   Embodiment 64. The use of any one of embodiments 60-62, wherein said MORV is a recombinant MORV
   Embodiment 65. The use of embodiment 64, wherein said recombinant MORV comprises:
(a) a leader sequence of a first Vesiculovirus species different from said MORV;
(b) a synthetic intergenic region; and
(c) a trailer sequence of a second Vesiculovirus species different from said MORV.
   Embodiment 66. The recombinant MORV of embodiment 65, wherein said first Vesiculovirus species is a VSV.
   Embodiment 67. The recombinant MORV of embodiment 65, wherein said leader sequence comprises the sequence set forth in SEQ ID NO:3.
   Embodiment 68. The recombinant MORV of embodiment 67, wherein said leader sequence consists of said sequence set forth in SEQ ID NO:3.
   Embodiment 69. The recombinant MORV of any one of embodiments 65-68, wherein said leader sequence is located before a MORV-N gene within the MORV genome.
   Embodiment 70. The recombinant MORV of embodiment 65, wherein said synthetic intergenic region comprises a sequence set forth in SEQ ID NO:1 or SEQ ID NO:2.
   Embodiment 71. The recombinant MORV of embodiment 70, wherein said synthetic intergenic region consists of said sequence set forth in SEQ ID NO:2.
   Embodiment 72. The recombinant MORV of any one of embodiments 65-71, wherein said synthetic intergenic region is located after a stop of a first gene within said MORV genome and before a start codon of a second gene within said MORV genome.
   Embodiment 73. The recombinant MORV of any one of embodiments 65-71, where said synthetic intergenic region is located between each gene within the MORV genome.
   Embodiment 74. The recombinant MORV of embodiment 65, wherein said second Vesiculovirus species is a VSV.
   Embodiment 75. The recombinant MORV of embodiment 74, wherein said trailer sequence comprises a sequence set forth in SEQ ID NO:4.
   Embodiment 76. The recombinant MORV of embodiment 75, wherein said trailer sequence consists of said sequence set forth in SEQ ID NO:4.
   Embodiment 77. The recombinant MORV of any one of embodiments 75-76, wherein said trailer sequence is located after a MORV-L gene within the MORV genome.
   Embodiment 78. The recombinant MORV of any one of embodiments 65-77, wherein a genome of said recombinant MORV comprises:
(a) said leader sequence before a MORV-N gene within said genome;
(b) said synthetic intergenic region (i) after a stop codon of said MORV-N gene and before a start codon of a MORV-P gene, (ii) after a stop codon of said MORV-P gene and before a start codon of a MORV-M gene, (iii) after a stop codon of said MORV-M gene and before a start codon of a MORV-G gene, (iv) after a stop codon of said MORV-G gene and before a start codon of a MORV-L gene, and (v) after a stop codon of a MORV-L gene; and
(c) said trailer sequence after said MORV-L gene.
   Embodiment 79. The recombinant MORV of embodiment 65, wherein a genome of said recombinant MORV comprises the nucleic acid sequence set forth in SEQ ID NO:5.
   Embodiment 80. The recombinant MORV of embodiment 65, wherein a genome of said recombinant MORV comprises the nucleic acid sequence set forth in SEQ ID NO:6.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A recombinant Morreton virus (MORV), wherein the genome of said MORV comprises:
(a) a leader sequence of a first Vesiculovirus species different from said MORV;
(b) a synthetic intergenic region; and
(c) a trailer sequence of a second Vesiculovirus species different from said MORV.

2. The recombinant MORV of claim 1, wherein said first Vesiculovirus species is a vesicular stomatitis virus (VSV).

3. The recombinant MORV of claim 2, wherein said leader sequence comprises the sequence set forth in SEQ ID NO:3.

4. The recombinant MORV of claim 1, wherein said leader sequence is located before a MORV-N gene within the genome.

5. (canceled)

6. The recombinant MORV of claim 1, wherein said synthetic intergenic region is located after a stop of a first gene within said genome and before a start codon of a second gene within said genome.

7. (canceled)

8. The recombinant MORV of claim 1, wherein said second Vesiculovirus species is a VSV.

9. The recombinant MORV of claim 8, wherein said trailer sequence comprises a sequence set forth in SEQ ID NO:4.

10. The recombinant MORV of claim 9, wherein said trailer sequence is located after a MORV-L gene within the genome.

11. (canceled)

12. The recombinant MORV of claim 1, wherein said genome comprises the nucleic acid sequence set forth in SEQ ID NO:5 or SEQ ID NO:6.

13. A method for treating a mammal having cancer, wherein said method comprises administering, to said mammal, a composition comprising MORV, wherein the number of cancer cells within said mammal is reduced.

14. The method of claim 13, wherein said mammal is a human.

15-16. (canceled)

17. The method of claim 13, wherein said MORV is a wild-type MORV.

18. The method of claim 13, wherein said MORV is a recombinant MORV.

19. The method of claim 18, wherein the genome of said recombinant MORV comprises:
(a) a leader sequence of a first Vesiculovirus species different from said MORV;
(b) a synthetic intergenic region; and
(c) a trailer sequence of a second Vesiculovirus species different from said MORV.

20. The method of claim 19, wherein said first Vesiculovirus species is a VSV.

21. The method of claim 20, wherein said leader sequence comprises the sequence set forth in SEQ ID NO:3.

22-23. (canceled)

24. The method of claim 19, wherein said synthetic intergenic region is located after a stop of a first gene within said genome and before a start codon of a second gene within said genome.

25. (canceled)

26. The method of claim 19, wherein said second Vesiculovirus species is a VSV.

27. The method of claim 26, wherein said trailer sequence comprises a sequence set forth in SEQ ID NO:4.

28-29. (canceled)

30. The method of claim 19, wherein said genome comprises the nucleic acid sequence set forth in SEQ ID NO:5 or SEQ ID NO:6.

31-38. (canceled)


SEQUENCE LISTING (16 sequences)

SEQ ID NO: 1 - skipped

SEQ ID NO: 2 - DNA, 11 bp, synthetic construct
  source 1..11: mol_type = other DNA
tatgaaaaaaa

SEQ ID NO: 3 - DNA, 59 bp, synthetic construct
  source 1..59: mol_type = other DNA
agacaaacaaaccattattacaattaaaaggctcaggagaaccttcaacagcaatcgaa

SEQ ID NO: 4 - DNA, 72 bp, synthetic construct
  source 1..72: mol_type = other DNA
gataagacttagaaccctcttaggattttttttgttttaaatggtttgttggtttggcatggcatctccacc

SEQ ID NO: 5 - DNA, 11181 bp, Morreton virus
  source 1..11181: mol_type = genomic DNA
agacaaacaaaccattattacaattaaaaggctcaggagaaccttcaacagcaatcgaaatgtcggttacagtcaaaagaatcattgacaactctgtcatcctccccaaattgccagccaatgaggatccagttgaatatccgggtgactatttcaaaaagacaaatgaggtccctgtgtacatcaacaccagcaagactctaaatgatctgagagggtatgtttatcaaggactaaaaacaggcaatgtgtctataatccatgtcaacagctatctgtatgcagcattaagagacattaaaggaaaactcgacaaagattggatcagcttcggagttcagatcgggaaaaccggagatgaagttggaatattcaatttagtgtcagtcaaaactctggaaggaattattccagatggagtatcagatgcatcaagaactagcgcagatgatgcctggcttcccctttatttactggggctttacagagtggggaggactcaaatgccagaataccggaagaaattgatggatggtctaattaatcagtgcaaaatgatcaatgagaagttcgaacctctagttcctgaggggcgtgacatctttgatgtgtggggaaacgacagcaattataccaaaatcgtagccgccgtcgacatgttctttcatatgttcaaaaagcatgagaaggcctctttcagatatggaactatagtgtccaggttcaaggactgcgccgcattggcgacatttggacatctatgtaaaatcactggcatgtctacagaagatgtgaccacttggatcctcaatagagaagtggcagatgaaatggttcaaatgatgcttccaggccaagaaattgataaggcggattcttacatgccttacctcatagacttcggactgtcttcaaagtctccatactcgtccgtaaaaaacccggctttccacttttggggtcaattgactgcattgctgttgagatcaactcgagcaaggaatgccagacaacctgacgacatcgagtatacatcattgacaacagcaggtctcctatatgcttatgcagtaggttcatcagcagaccttgcgcagcaattttgcattggggacaacaaatacgttcctgaccccaatgcagggggactcacaacgaatgctcctcctcaaggacgagatgttgtagaatggctaggttggtttgaagaccagaatagaaagcccaccccagacatgttgctatatgcaaagagagcagtcaactctctgcaagggttaagagaaaaaacagtcgggaagtatgccaaatctgagtttgacaagtgaccatgtgttgacagcctatactataagatctgttacatatgaaaaaaactaacagaaatcatggacaatctggccaaagttagagaatatcttaagacatattcccgtttagaccaagcagttcaagagatggacgatttagaagctcagagagaggagaagactaactatgaattatttcaagaagaagggatagatattcagaatcatccatcttattatcaggcagcagcagatgaaagttcagatagtgaagaagaagagatgttggaagcttatgatgtcactgaggaccaacataagactatttctacagatgaggtagagggttatattgctgaaccatctgacgattacgcagatgatgaagtcaacgtagttttcacctcagattggaaacaaccagagctagaatctgatggaaatgggaagacactgaggctcacaatgccagaaggcttaagcaatgaacagcaatcccaatggctttcaaccattaaagctgttgtgcagagtgcaaagtattggaatattgcagagtgtaccctggagagcacaaagtcaggggttgtaatgaaagaacgacaaatgacccctgatgtatataaggtgacccctgtcctcaacaatccttcatcgatagaagagactccaacagatgtttggtccctttgtcaggtttctgtttctttcaccccgaggaaaagtggcattcagccatttgtaatctctctggaggaattatttaactcacgtgcagaattcatctcagttggcggcaacggaaaaatgtcacataaagaggcaatactccttgggctccgttataaaaagctatacaatcaagcaagagtgaagtataatctaggatgattatgaaaaaaactaacagagatacaaatgctctgaatacttaagtgttcaaaatgagctccctcaagaaaatacttggtattaagggaaagaacaaaaaatctaagaaattgggccttcctcctcctccttatgaagaagatgcaaggatggaatttgcacctagtgccccaattgacagatcatttttcggagttgaagatatggatatccaggataaaaagcaactcagatacgagaagttctatttttcagtcaagatgacagttcgctcaaatagacccttcagaacttactctgatgtcgcatcggctgtatctaactgggatcacatgtatattgggatggcaggaaaaagaccattttataaaatcttggccttcttgggatctacactcttgaaagcaactccagccgtgctagccgaccacggacagccagaatatcacgcccattgtgaaggccgagcttatctccctcatcgattaggtccaactccccccatgttgaacgtccctgaacattttcggagaccttttaatataggtctgttcagaggcactattgatctaacaatgactctccatgatgatgagtctttggaggccgcaccaatgatttgggatcattttaatgcatcccgagtcactgattttccagaaaaggccttgctatttggactgattgtagagaagaaagcaacgggggcttggatcttagactcaatcagcaatttcaaataatcttctttgacaaattctcagctacataatttattgtgaatcagtcttgcttaccttatgaaaaaaactaacagaaatcattctgtttcgtcactatgctggttttatacctgttattgagccttttggctctgggagctcaatgcaagttcactatagtatttcctcacaatcaaaaagggaattggaaaaatgtaccggcaaattatcagtattgtccttctagttctgacttgaattggcacaatgggctgattggcacttctctccaagtcaaaatgcccaaaagccataaggccatccaagcggatggttggatgtgtcatgctgccaagtgggtgactacttgtgacttcagatggtacggacctaaatatgtgacacattctataaagtccatgatacctacagtcgaccagtgtaaagaaagtatagcccagactaaacaaggaacgtggttaaatccgggtttccctccccaaagttgtggatatgcttccgttacagatgcagaggctgtgatagtcaaagcaaccccccaccaggttttggttgacgaatatacaggagaatgggttgactcccaatttccgactggaaaatgcaataaagacatttgcccaacagttcacaactcaactacctggcactcagattataaggtcactggcctttgcgatgcaaatttgatctcaatggacatcactttcttctccgaagatggaaaattaacatccctcgggaaagaaggaacagggttcagaagcaattactttgcatacgaaaatggtgacaaagcatgccgcatgcagtactgtaaacactggggagttcgacttccatccggagtgtggttcgaaatggcagataaagacatctataatgatgcgaaattcccggattgccctgaaggatcatccattgcggctccctctcagacttcagtcgatgttagtctcattcaggatgtagagagaatcttggactactctttgtgtcaggaaacctggagcaaaattcgtgctcatttgcccatttcaccagttgacctcagctatttatccccaaaaaatcctggaactggtcctgcattcactatcatcaatgggacattaaaatactttgagactcgatacataagagtcgatatcgcaggacccatcattcctcaaatgagaggagtaatcagcggaaccacgaccgagagagagctgtggacggactggtacccctacgaagatgttgaaatcggaccaaatggggttttgaaaactgctacagggtataagttccctttatatatgattggccacggcatgctcgactcagatctccacatcacatcaaaggctcaggtttttgaacatccccatattcaggatgctgcttctcagcttcctgatgatgagactttattttttggtgatactggactctcgaaaaaccccatagagcttgtagaaggttggttcagcggatggaaaagcactattgcttcttttttcttcataatagggcttgtgatcggattatatttggttcttaggattggaatcgctttatgcatcaaatgccgagtgcaggagaaaaggcccaaaatttacactgatgtggaaatgaacagattggatcgatgaaagcttcattgctgcagatgacaaccacactcaccaaagatctccagtttcaaagttatattggagacacaattttacaataatgatgactacaggaaaccaaattataatcacttccaaagtaaacacgacttaattttatgtatgaaaaaaactaacagccatcatggatgtcaacgattttgagattgatgatcaggtcacatttgatgaggaagattatgttacacaggaattcctcaatcctgatgagaggatgacctatcttaatcatgcagattacaatctgaattcccccttgattagtgatgacatcgacaatctgttaagaagatataacagcatgccggtaccaaaaatgtgggaaaacaagccatgggagggagtgttggaaatgctgacctcttgtcaagctaaccctttgccatccactaaaattcataattggatgggaagatggctcatgtcagatgaccacgataccagccaaggttatagctttctccatgaagttgataaggaagcagagttgaccttcgacattgtggagactttcttgaaaggatggggaggatgtgtaataaaatttcaaaagaaggaaggttttcatgatgctttcaaggtcgttgcatcattgtgccaaaaattcttagacctacataaattaactctcatcttgaatgctgtctctcaagacgaactgaataatctaagcagaacattcaaagggaaaaatagaacagccaattcaggaaacattatcaccagattgagagtaccaagtttgggacctgcttttgtgacactgggctgggtgtacttaaaaaagttagatctcttatttgatcggaatgtgctacttatgatcaaagatgttatcattggacggatgcagactattctgtccatgattggaaggaatgatgccttattttcagaacaagacatattcacattgttgacaatttatagaattggagatcgcatcgtcgaaagattgggaaatcaagcgtatgacttaatcaaaatggttgaacctatatgtaatttaaaactgatgaacttggctcgagagtatcgcccgctagttccaaaattccctcactttgaacaacacattcttcaatctgttcaagaagccaagaaaatagataatggaatcagttatctgcatgatcagatcttaagtctccagtctgtagatctgactctcgtcgtctatggatctttccgacattggggtcatccattcattgattattatgcaggccttgaaaaacttcataagcaggtcacaatggaaaagacaattgactcatcttacgccaatgccctcgctagtgacctagcccggactgtcctacagaatcaattcaatgagcacaaaaaatggtttgtagatgaaggcttgataagtgctgatcatcccttcaaaaatcacattctggaaaacacctggccgactgcagctcaagtgcaggattttggggataagtggcatcatttgccccttattaagtgttttgaaattccagatctcttagatccgtcaatcatctattcagataaaagtcactctatgaataaaagcgaagttcttcgtcatgttagactcaatcccacaactccaatacccagcaggaaagttttgcagacaatgctagatacaaaggcgacagattggaaggagtttttgcaaaacattgatgaacacgggttagatgaggatgatctagtaataggtctcaaaggcaaagagcgagaactcaaactagccggtcgatttttctcattaatgtcatggagattacgtgagtattttgtgatcacagagtacctgatcaagacccattttgttcctatgtttaaagggttgactatggcagatgatttgacagctgtcataaaaaaaatgttagactcttcttcaggccagggacttgataattatgaagcaatttgcatagccaatcatattgattacgaaaaatggaacaaccatcaacggaaggaatcaaatggtcctgtgttccgtgtcatgggccagtttttaggttatccatctttgattgagagaactcacgaattctttgagaaaagcttgatttattacaacggaaggccggatttgatgagggtatcgaacaatacacttgttaatgctacgaaccaaagagtctgttggcagggacaagcaggaggtttagaaggtcttagacaaaaaggatggagtatactgaatctgcttgtcatacaaagagaggcaaaaatccgcaacactgcagtaaaagttttagcacaaggagataatcaagttatttgcactcagtataagacaaaaaaatcgagaaacgacctggagctacgatctgcgttaaatcagatggtattgaacaatgaacacatcatgaacgccatcaaagcaggaacaggtcgtcttggacttgtgatcaatgatgacgaaaccatgcagtcagctgattaccttaattacggaaaaattccaatctttcgtggcgtaattcgaggactcgaaactaaaagatggtcccgtgtaacatgtgttactaacgatcagatccccacatgtgccaatatcatgagctctgtttcaacaaatgcattgaccgtagcacattttgccgagaatcccatcaatgcaatgatacaatataattatttcgggaattttgcacgactcttattgatgatgcacgatccagcacttcgtaagtctctatatgaggtacagtcttcaattccaggattacatagcgtcacattcaagtatgctatgttatatctagatccatccattggaggagtatccgggatgtctttgtcgagattcttaattcgagctttccccgatccagttactgagagcttatcattttggaaattcattcatgatcacacaaaggaggaccacttaaaagagattagtgctgtattcgggaatccagacattgcacgattccgcctaacacacatagataaattagttgaggatcctacttcactaaatatcgcaatgggcatgagtcctgcaaacctcctcaaaacagaagtcaaaaaatgcttaattgaatccagacaatcaattaaaaatcaagtgatcaaagacgccacaatttatctataccacgaagaagataagctcaggagtttcctatggtctatcaatcccttatttcctcgcttcttaagcgaattcaaatcaggaacatttttgggtgtcgcagatggtctgatcagtttgttccaaaattcaagaacaattcgaaattcttttagaagaaaatatcatagggagcttgatgatctgataatcaaaagtgaggtctcatcactcatacacttaggaaagctacatcttcgaagaggcgcttatcgaatttggagctgctcctcaacccaggctgacaccctccgttataaatcatggggtcgtacagtcataggcacaactgtccctcatcctctggaaatgttaggacctcattcaaagaaagaaggaccctgtgtcgcctgtaatgcatcaggttttaattatgtctcagtgtattgtccttctggaatcaataatgtcttcttatcccgaggtccgctaccagcctatttgggttctaaaacctctgaatcaacttccattttgcaaccttgggaacgtgagagcaaagtaccactgataaagagagccactagattaagagatgctatctcatggtttgttgaccccaattctaatttggcaaaaactatccttgataatattcatgcattaacaggcgaggaatggtcaaagaaacaacatggattcaaacggacaggatctgcattgcatcgattctctacctcaagaatgagccatggcgggtttgcctcacagagcactgcagcattgacaagattaatggcgaccacagacacgatgagggacttaggagatcagaactatgattttctctttcaagccactcttctatatgctcaaatcacaacaaccgtggttcgaaacggttatctctccagttgcaccgatcactatcacattacttgccggtcatgcctcaggaccatagaggaggttacattggattctactatggattactcacctccagacgtttcccatgtcttgaagacttggagaaatggggaaggatcctggggacaagaaattaaacaaatatatcctgtggagggggattggaaaactttatctcctgcagagcaatcttatcaagtgggaagatgcatcggattcctttatggggacttagcatatagaaagtcatctcatgcggatgacagctctctttttcccctttccatacaaaatagaatccgtggaagaggtttcctcaaaggactccttgatggattaatgagggcgagttgttgtcaggtgattcatcgcagaagtttagcccatctaaaacggccagctaacgcagtgtatggaggtctgatttatctcatcgataagattagtgcttctgcaccattcttgtctttgaccagatctggtcctttacgaagtgagttggaaactgttcctcacaagatacccacttcttatccaaccagcaatagagatatgggaataattgttcgaaattattttaagtaccaatgcagacagattgaaaagggcaagtacaagacacactacacacaattatggttgttttctgatgtactatccatcgattttcttggtcctttatccatatctacaatcctaatgactctactgtacaaacaatctctttctgcaagggacaaaaatgaactacgtgaattagctaatttgtcatcattattgagatcaggggaaggctgggaagaagttcatgtcaaattcttttctagggatatattactttgtccagaagaaatcagacatgcctgcaaatttggtatcgcgaaagaaataaacacagagtcctattaccctccttgggaaaaggaagtaacaggacctataacaattcatcctatctattacacaacggttcctcatccgaagatcttagattcgcctccgcgggtgcaaaaccctttgttatctggactaagattaggtcaactaccaacaggagcacattacaaaattcgaagcattattcgaggattaaaaatacacttcaaagacgtcttatgttgcggagatggatcaggaggtatgacagcagctttgttgagagaaagtcgacacagtagggcaattttcaatagtttactggaattgtccggttccataatgaggggtgcttcaccagagcctcctagtgctctggaaaccttgggtgatgaaaaacgaagatgtgtaaatggatcaacttgctgggaacatccatctgatttaagtgacacgaaaacatgggattattttcttcaattgaaaaatggattagggcttcagctcgacttaattgtgatggatatggaggttagagatccaataattagtcaaaaaatagaatataatgttcgccattatatgcatcgactgctagatcagaatggagtcttaatttacaagacatatggcacctacctccaatccatcactcagaacattctaacaattgccggaccactatttaggtctgttgatttggttcagactgagttcagtagttcccaaacctctgaagtgtattgtgtctgccagggattaaaaaacatgatagatgaacctcatgtagattggtcattgctgagagatagatggaatcagctgtatgcattccaaacagaggaacaggaattcatcagagccaagaaaatgtgtcagagagatactttgaccggcatcccagctcaattcatcccagacccgtttgtcaacttagaaacaatcttgcaaattttcggagtccctacaggggttgcgcattcggccgcacttactgcatcctcacatccgaatgagttagtgacagttagtctcttttttatgacaataatatcatattacaatctcaaccacctaaggaaatcgcctagtgttcctccccctccatcagatggaatagctcaaaatgttggtgtctctttcgtcgggataagtttatggttaagtctcattgaattggaccttaaactgtataaaagatcattaagagctataagaacttctttcccgattagattggagacagtcagagtacttgacggttacaaacttagatggcatgtccatggattgggaatccccaaagattgtcgaatctctgactcattagctgcaattggcaactggattcgagctttagaacttatccggaatcaatcaaatcagaagccattctgtgagcgcctgttcaaccagctttgtcgtttagtagactaccatctgaaatggtctacactgagagaccagacgggtgtcagggattggttaaccggacatgtgtccattaatgataaatctattttaatcacaaggagtgacgtacatgatgagaactcttggagatcttaaagatgtctatatcaatttgaagagcaaaagctacagtatgaaaaaaactgataagacttagaaccctcttaggattttttttgttttaaatggtttgttggttt

SEQ ID NO: 6 - DNA, 11197 bp, synthetic construct
  source 1..11197: mol_type = other DNA
agacaaacaaaccattattacaattaaaaggctcaggagaaccttcaacagcaatcgaaatgtcggttacagtcaaaagaatcattgacaactctgtcatcctccccaaattgccagccaatgaggatccagttgaatatccgggtgactatttcaaaaagacaaatgaggtccctgtgtacatcaacaccagcaagactctaaatgatctgagagggtatgtttatcaaggactaaaaacaggcaatgtgtctataatccatgtcaacagctatctgtatgcagcattaagagacattaaaggaaaactcgacaaagattggatcagcttcggagttcagatcgggaaaaccggagatgaagttggaatattcaatttagtgtcagtcaaaactctggaaggaattattccagatggagtatcagatgcatcaagaactagcgcagatgatgcctggcttcccctttatttactggggctttacagagtggggaggactcaaatgccagaataccggaagaaattgatggatggtctaattaatcagtgcaaaatgatcaatgagaagttcgaacctctagttcctgaggggcgtgacatctttgatgtgtggggaaacgacagcaattataccaaaatcgtagccgccgtcgacatgttctttcatatgttcaaaaagcatgagaaggcctctttcagatatggaactatagtgtccaggttcaaggactgcgccgcattggcgacatttggacatctatgtaaaatcactggcatgtctacagaagatgtgaccacttggatcctcaatagagaagtggcagatgaaatggttcaaatgatgcttccaggccaagaaattgataaggcggattcttacatgccttacctcatagacttcggactgtcttcaaagtctccatactcgtccgtaaaaaacccggctttccacttttggggtcaattgactgcattgctgttgagatcaactcgagcaaggaatgccagacaacctgacgacatcgagtatacatcattgacaacagcaggtctcctatatgcttatgcagtaggttcatcagcagaccttgcgcagcaattttgcattggggacaacaaatacgttcctgaccccaatgcagggggactcacaacgaatgctcctcctcaaggacgagatgttgtagaatggctaggttggtttgaagaccagaatagaaagcccaccccagacatgttgctatatgcaaagagagcagtcaactctctgcaagggttaagagaaaaaacagtcgggaagtatgccaaatctgagtttgacaagtgaccatgtgttgacagcctatactataagatctgttacatatgaaaaaaactaacagaaatcatggacaatctggccaaagttagagaatatcttaagacatattcccgtttagaccaagcagttcaagagatggacgatttagaagctcagagagaggagaagactaactatgaattatttcaagaagaagggatagatattcagaatcatccatcttattatcaggcagcagcagatgaaagttcagatagtgaagaagaagagatgttggaagcttatgatgtcactgaggaccaacataagactatttctacagatgaggtagagggttatattgctgaaccatctgacgattacgcagatgatgaagtcaacgtagttttcacctcagattggaaacaaccagagctagaatctgatggaaatgggaagacactgaggctcacaatgccagaaggcttaagcaatgaacagcaatcccaatggctttcaaccattaaagctgttgtgcagagtgcaaagtattggaatattgcagagtgtaccctggagagcacaaagtcaggggttgtaatgaaagaacgacaaatgacccctgatgtatataaggtgacccctgtcctcaacaatccttcatcgatagaagagactccaacagatgtttggtccctttgtcaggtttctgtttctttcaccccgaggaaaagtggcattcagccatttgtaatctctctggaggaattatttaactcacgtgcagaattcatctcagttggcggcaacggaaaaatgtcacataaagaggcaatactccttgggctccgttataaaaagctatacaatcaagcaagagtgaagtataatctaggatgattatgaaaaaaactaacagagatccaaatgctctgaatacttaagtgttcaaaatgagctccctcaagaaaatacttggtattaagggaaagaacaaaaaatctaagaaattgggccttcctcctcctccttatgaagaagatgcaaggatggaatttgcacctagtgccccaattgacagatcatttttcggagttgaagatatggatatccaggataaaaagcaactcagatacgagaagttctatttttcagtcaagatgacagttcgctcaaatagacccttcagaacttactctgatgtcgcatcggctgtatctaactgggatcacatgtatattgggatggcaggaaaaagaccattttataaaatcttggccttcttgggatctacactcttgaaagcaactccagccgtgctagccgaccacggacagccagaatatcacgcccattgtgaaggccgagcttatctccctcatcgattaggtccaactccccccatgttgaacgtccctgaacattttcggagaccttttaatataggtctgttcagaggcactattgatctaacaatgactctccatgatgatgagtctttggaggccgcaccaatgatttgggatcattttaatgcatcccgagtcactgattttccagaaaaggccttgctatttggactgattgtagagaagaaagcaacgggggcttggatcttagactcaatcagcaatttcaaataatcttctttgacaaattctcagctacataatttattgtgaatcagtcttgcttaccttatgaaaaaaactaacagaaatcattctgtttcgtcactatgctggttttatacctgttattgagccttttggctctgggagctcaatgcaagttcactatagtatttcctcacaatcaaaaagggaattggaaaaatgtaccggcaaattatcagtattgtccttctagttctgacttgaattggcacaatgggctgattggcacttctctccaagtcaaaatgcccaaaagccataaggccatccaagcggatggttggatgtgtcatgctgccaagtgggtgactacttgtgacttcagatggtacggacctaaatatgtgacacattctataaagtccatgatacctacagtcgaccagtgtaaagaaagtatagcccagactaaacaaggaacgtggttaaatccgggtttccctccccaaagttgtggatatgcttccgttacagatgcagaggctgtgatagtcaaagcaaccccccaccaggttttggttgacgaatatacaggagaatgggttgactcccaatttccgactggaaaatgcaataaagacatttgcccaacagttcacaactcaactacctggcactcagattataaggtcactggcctttgcgatgcaaatttgatctcaatggacatcactttcttctccgaagatggaaaattaacatccctcgggaaagaaggaacagggttcagaagcaattactttgcatacgaaaatggtgacaaagcatgccgcatgcagtactgtaaacactggggagttcgacttccatccggagtgtggttcgaaatggcagataaagacatctataatgatgcgaaattcccggattgccctgaaggatcatccattgcggctccctctcagacttcagtcgatgttagtctcattcaggatgtagagagaatcttggactactctttgtgtcaggaaacctggagcaaaattcgtgctcatttgcccatttcaccagttgacctcagctatttatccccaaaaaatcctggaactggtcctgcattcactatcatcaatgggacattaaaatactttgagactcgatacataagagtcgatatcgcaggacccatcattcctcaaatgagaggagtaatcagcggaaccacgaccgagagagagctgtggacggactggtacccctacgaagatgttgaaatcggaccaaatggggttttgaaaactgctacagggtataagttccctttatatatgattggccacggcatgctcgactcagatctccacatcacatcaaaggctcaggtttttgaacatccccatattcaggatgctgcttctcagcttcctgatgatgagactttattttttggtgatactggactctcgaaaaaccccatagagcttgtagaaggttggttcagcggatggaaaagcactattgcttcttttttcttcataatagggcttgtgatcggattatatttggttcttaggattggaatcgctttatgcatcaaatgccgagtgcaggagaaaaggcccaaaatttacactgatgtggaaatgaacagattggatcgatgaaagcttcattgctgcagatgacaaccacactcaccaaagatctccagtttcaaagttatattggagacacaattttacaataatgatgactacaggaaaccaaattataatcacttccaaagtaaacacgacttaattttatgtatgaaaaaaactaacagccatcatggatgtcaacgattttgagattgatgatcaggtcacatttgatgaggaagattatgttacacaggaattcctcaatcctgatgagaggatgacctatcttaatcatgcagattacaatctgaattcccccttgattagtgatgacatcgacaatctgttaagaagatataacagcatgccggtaccaaaaatgtgggaaaacaagccatgggagggagtgttggaaatgctgacctcttgtcaagctaaccctttgccatccactaaaattcataattggatgggaagatggctcatgtcagatgaccacgataccagccaaggttatagctttctccatgaagttgataaggaagcagagttgaccttcgacattgtggagactttcttgaaaggatggggaggatgtgtaataaaatttcaaaagaaggaaggttttcatgatgctttcaaggtcgttgcatcattgtgccaaaaattcttagacctacataaattaactctcatcttgaatgctgtctctcaagacgaactgaataatctaagcagaacattcaaagggaaaaatagaacagccaattcaggaaacattatcaccagattgagagtaccaagtttgggacctgcttttgtgacactgggctgggtgtacttaaaaaagttagatctcttatttgatcggaatgtgctacttatgatcaaagatgttatcattggacggatgcagactattctgtccatgattggaaggaatgatgccttattttcagaacaagacatattcacattgttgacaatttatagaattggagatcgcatcgtcgaaagattgggaaatcaagcgtatgacttaatcaaaatggttgaacctatatgtaatttaaaactgatgaacttggctcgagagtatcgcccgctagttccaaaattccctcactttgaacaacacattcttcaatctgttcaagaagccaagaaaatagataatggaatcagttatctgcatgatcagatcttaagtctccagtctgtagatctgactctcgtcgtctatggatctttccgacattggggtcatccattcattgattattatgcaggccttgaaaaacttcataagcaggtcacaatggaaaagacaattgactcatcttacgccaatgccctcgctagtgacctagcccggactgtcctacagaatcaattcaatgagcacaaaaaatggtttgtagatgaaggcttgataagtgctgatcatcccttcaaaaatcacattctggaaaacacctggccgactgcagctcaagtgcaggattttggggataagtggcatcatttgccccttattaagtgttttgaaattccagatctcttagatccgtcaatcatctattcagataaaagtcactctatgaataaaagcgaagttcttcgtcatgttagactcaatcccacaactccaatacccagcaggaaagttttgcagacaatgctagatacaaaggcgacagattggaaggagtttttgcaaaacattgatgaacacgggttagatgaggatgatctagtaataggtctcaaaggcaaagagcgagaactcaaactagccggtcgatttttctcattaatgtcatggagattacgtgagtattttgtgatcacagagtacctgatcaagacccattttgttcctatgtttaaagggttgactatggcagatgatttgacagctgtcataaaaaaaatgttagactcttcttcaggccagggacttgataattatgaagcaatttgcatagccaatcatattgattacgaaaaatggaacaaccatcaacggaaggaatcaaatggtcctgtgttccgtgtcatgggccagtttttaggttatccatctttgattgagagaactcacgaattctttgagaaaagcttgatttattacaacggaaggccggatttgatgagggtatcgaacaatacacttgttaatgctacgaaccaaagagtctgttggcagggacaagcaggaggtttagaaggtcttagacaaaaaggatggagtatactgaatctgcttgtcatacaaagagaggcaaaaatccgcaacactgcagtaaaagttttagcacaaggagataatcaagttatttgcactcagtataagacaaaaaaatcgagaaacgacctggagctacgatctgcgttaaatcagatggtattgaacaatgaacacatcatgaacgccatcaaagcaggaacaggtcgtcttggacttgtgatcaatgatgacgaaaccatgcagtcagctgattaccttaattacggaaaaattccaatctttcgtggcgtaattcgaggactcgaaactaaaagatggtcccgtgtaacatgtgttactaacgatcagatccccacatgtgccaatatcatgagctctgtttcaacaaatgcattgaccgtagcacattttgccgagaatcccatcaatgcaatgatacaatataattatttcgggaattttgcacgactcttattgatgatgcacgatccagcacttcgtaagtctctatatgaggtacagtcttcaattccaggattacatagcgtcacattcaagtatgctatgttatatctagatccatccattggaggagtatccgggatgtctttgtcgagattcttaattcgagctttccccgatccagttactgagagcttatcattttggaaattcattcatgatcacacaaaggaggaccacttaaaagagattagtgctgtattcgggaatccagacattgcacgattccgcctaacacacatagataaattagttgaggatcctacttcactaaatatcgcaatgggcatgagtcctgcaaacctcctcaaaacagaagtcaaaaaatgcttaattgaatccagacaatcaattaaaaatcaagtgatcaaagacgccacaatttatctataccacgaagaagataagctcaggagtttcctatggtctatcaatcccttatttcctcgcttcttaagcgaattcaaatcaggaacatttttgggtgtcgcagatggtctgatcagtttgttccaaaattcaagaacaattcgaaattcttttagaagaaaatatcatagggagcttgatgatctgataatcaaaagtgaggtctcatcactcatacacttaggaaagctacatcttcgaagaggcgcttatcgaatttggagctgctcctcaacccaggctgacaccctccgttataaatcatggggtcgtacagtcataggcacaactgtccctcatcctctggaaatgttaggacctcattcaaagaaagaaggaccctgtgtcgcctgtaatgcatcaggttttaattatgtctcagtgtattgtccttctggaatcaataatgtcttcttatcccgaggtccgctaccagcctatttgggttctaaaacctctgaatcaacttccattttgcaaccttgggaacgtgagagcaaagtaccactgataaagagagccactagattaagagatgctatctcatggtttgttgaccccaattctaatttggcaaaaactatccttgataatattcatgcattaacaggcgaggaatggtcaaagaaacaacatggattcaaacggacaggatctgcattgcatcgattctctacctcaagaatgagccatggcgggtttgcctcacagagcactgcagcattgacaagattaatggcgaccacagacacgatgagggacttaggagatcagaactatgattttctctttcaagccactcttctatatgctcaaatcacaacaaccgtggttcgaaacggttatctctccagttgcaccgatcactatcacattacttgccggtcatgcctcaggaccatagaggaggttacattggattctactatggattactcacctccagacgtttcccatgtcttgaagacttggagaaatggggaaggatcctggggacaagaaattaaacaaatatatcctgtggagggggattggaaaactttatctcctgcagagcaatcttatcaagtgggaagatgcatcggattcctttatggggacttagcatatagaaagtcatctcatgcggatgacagctctctttttcccctttccatacaaaatagaatccgtggaagaggtttcctcaaaggactccttgatggattaatgagggcgagttgttgtcaggtgattcatcgcagaagtttagcccatctaaaacggccagctaacgcagtgtatggaggtctgatttatctcatcgataagattagtgcttctgcaccattcttgtctttgaccagatctggtcctttacgaagtgagttggaaactgttcctcacaagatacccacttcttatccaaccagcaatagagatatgggaataattgttcgaaattattttaagtaccaatgcagacagattgaaaagggcaagtacaagacacactacacacaattatggttgttttctgatgtactatccatcgattttcttggtcctttatccatatctacaatcctaatgactctactgtacaaacaatctctttctgcaagggacaaaaatgaactacgtgaattagctaatttgtcatcattattgagatcaggggaaggctgggaagaagttcatgtcaaattcttttctagggatatattactttgtccagaagaaatcagacatgcctgcaaatttggtatcgcgaaagaaataaacacagagtcctattaccctccttgggaaaaggaagtaacaggacctataacaattcatcctatctattacacaacggttcctcatccgaagatcttagattcgcctccgcgggtgcaaaaccctttgttatctggactaagattaggtcaactaccaacaggagcacattacaaaattcgaagcattattcgaggattaaaaatacacttcaaagacgtcttatgttgcggagatggatcaggaggtatgacagcagctttgttgagagaaagtcgacacagtagggcaattttcaatagtttactggaattgtccggttccataatgaggggtgcttcaccagagcctcctagtgctctggaaaccttgggtgatgaaaaacgaagatgtgtaaatggatcaacttgctgggaacatccatctgatttaagtgacacgaaaacatgggattattttcttcaattgaaaaatggattagggcttcagctcgacttaattgtgatggatatggaggttagagatccaataattagtcaaaaaatagaatataatgttcgccattatatgcatcgactgctagatcagaatggagtcttaatttacaagacatatggcacctacctccaatccatcactcagaacattctaacaattgccggaccactatttaggtctgttgatttggttcagactgagttcagtagttcccaaacctctgaagtgtattgtgtctgccagggattaaaaaacatgatagatgaacctcatgtagattggtcattgctgagagatagatggaatcagctgtatgcattccaaacagaggaacaggaattcatcagagccaagaaaatgtgtcagagagatactttgaccggcatcccagctcaattcatcccagacccgtttgtcaacttagaaacaatcttgcaaattttcggagtccctacaggggttgcgcattcggccgcacttactgcatcctcacatccgaatgagttagtgacagttagtctcttttttatgacaataatatcatattacaatctcaaccacctaaggaaatcgcctagtgttcctccccctccatcagatggaatagctcaaaatgttggtgtctctttcgtcgggataagtttatggttaagtctcattgaattggaccttaaactgtataaaagatcattaagagctataagaacttctttcccgattagattggagacagtcagagtacttgacggttacaaacttagatggcatgtccatggattgggaatccccaaagattgtcgaatctctgactcattagctgcaattggcaactggattcgagctttagaacttatccggaatcaatcaaatcagaagccattctgtgagcgcctgttcaaccagctttgtcgtttagtagactaccatctgaaatggtctacactgagagaccagacgggtgtcagggattggttaaccggacatgtgtccattaatgataaatctattttaatcacaaggagtgacgtacatgatgagaactcttggagatcttaagatgtctatatcaatttgaagagcaaaagctacagtatgaaaaaaactgataagacttagaaccctcttaggattttttttgttttaaatggtttgttggtttggcatggcatctccacc

SEQ ID NO: 7 - DNA, 24 bp, synthetic construct
  source 1..24: mol_type = other DNA
tgatagtaccggaggattgacgac

SEQ ID NO: 8 - DNA, 22 bp, synthetic construct
  source 1..22: mol_type = other DNA
ctgcatcatatcaggagtcggt

SEQ ID NO: 9 - DNA, 30 bp, synthetic construct
  source 1..30: mol_type = other DNA
tcgaccacatctctgccttgtggcggtgca

SEQ ID NO: 10 - DNA, 24 bp, synthetic construct
  source 1..24: mol_type = other DNA
ccccaatgcagggggactcacaac

SEQ ID NO: 11 - DNA, 22 bp, synthetic construct
  source 1..22: mol_type = other DNA
tagcaacatgtctggggtgggc

SEQ ID NO: 12 - DNA, 30 bp, synthetic construct
  source 1..30: mol_type = other DNA
tctacaacatctcgtccttgaggaggagca

SEQ ID NO: 13 - DNA, 21 bp, synthetic construct
  source 1..21: mol_type = other DNA
tcgcaggacccatcattcctc

SEQ ID NO: 14 - DNA, 20 bp, synthetic construct
  source 1..20: mol_type = other DNA
caacatcttcgtaggggtac

SEQ ID NO: 15 - DNA, 30 bp, synthetic construct
  source 1..30: mol_type = other DNA
cggtcgtggttccgctgattactcctctca

SEQ ID NO: 16 - DNA, 10 bp, synthetic construct
  source 1..10: mol_type = other DNA
aacagcaatc